(19) United States
(12) Patent Application Publication
(10) Pub. No.: US 2008/0003224 A1
(43) Pub. Date: Jan. 3, 2008

(54) Compositions and methods for treating haematological proliferative disorders

(75) Inventors: Timothy C. Fong, Moraga, CA (US); Varghese Palath, Fremont, CA (US); R. J. Tesi, Berkeley, CA (US); Hoang Tran, Menlo Park, CA (US); Irving Weissman, Stanford, CA (US)

Correspondence Address: DORSEY & WHITNEY LLP, 555 CALIFORNIA STREET, SUITE 1000, SUITE 1000, SAN FRANCISCO, CA, 94104, US

(73) Assignee: Cellerant Therapeutics, Inc., San Carlos, CA (US)

(21) Appl. No.: 11/699,733

(22) Filed: Jan. 29, 2007

Related U.S. Application Data
(60) Provisional application No. 60/836,624, filed on Aug. 8, 2006.
(60) Provisional application No. 60/762,610, filed on Jan. 27, 2006.
(60) Provisional application No. 60/764,090, filed on Jan. 31, 2006.
(60) Provisional application No. 60/827,517, filed on Sep. 29, 2006.

(51) Int. Cl. A61K 39/395; A61K 39/00; A61P 35/00; C07K 16/00; C12N 5/00; C12P 21/08

(52) U.S. Cl. 424/139.1; 424/173.1; 435/346; 435/375; 530/387.1; 530/387.3

(57) ABSTRACT

The disclosure relates to antibodies immunoreactive with CD45RA+ hematopoietic tumor cells (HTCs), hybridomas producing such antibodies, and methods of using the antibodies, particularly as therapeutic treatments for conditions or diseases characterized by CD45RA+ HTCs.

### CROSS-REFERENCE TO RELATED APPLICATIONS

This application claims the benefit under 35 U.S.C. §119(e) of U.S. Ser. No. 60/762,610 filed Jan. 27, 2006, U.S. Ser. No. 60/764,090 filed Jan. 31, 2006, U.S. Ser. No. 60/836,624 filed Aug. 8, 2006, and U.S. Ser. No. 60/827,517 filed Sep. 29, 2006, each of which is hereby incorporated by reference in its entirety.

### 1. TECHNICAL FIELD

The present disclosure relates generally to antibodies capable of specifically binding to hematopoietic tumor cells and methods of using the antibodies, particularly as therapeutic treatments.

### 2. BACKGROUND

The cells of the hematopoietic system arise from multipotent progenitors, the hematopoietic stem cells (HSCs), which progress through a series of developmental programs to ultimately form the terminally differentiated cells of the myeloid or lymphoid lineage. It is believed that in the initial stages of hematopoiesis, HSCs commit to two distinguishable oligopotent but developmentally restricted progenitor cell types, the common lymphoid progenitors (CLPs) and the common myeloid progenitor (CMPs). T lymphocytes, B lymphocytes, natural killer (NK) cells, and lymphoid dendritic cells develop from corresponding progenitor cells derived from the CLPs whereas erythroid cells, megakaryocytes, granulocytes, macrophages, and myeloid dendritic cells develop from their corresponding progenitor cells derived from CMPs. Cell populations at each stage of differentiation are distinguishable from other cell populations in the hematopoietic pathway based on programmed expression of a unique set of cell markers.

Although HSCs are capable of self renewal—cell division that results in at least one of the daughter cells having the same characteristics as the parent cell—the progenitor cells committed to the lymphoid or myeloid lineages lose their potential to self-renew. That is, mitotic cell division of the committed progenitors leads to differentiated progeny rather than generation of a cell with the same proliferative and differentiation capacity as the parent cell. This loss of self-renewal potential is seen in the ability of committed progenitors cells to maintain hematopoiesis only for a limited time period (i.e., short term reconstitution) following transplantation of the progenitor cells into an immunocompromised animal, as compared to an HSC, which can completely regenerate and maintain hematopoiesis during the life of the host animal (i.e., long term reconstitution).

It has been observed, however, that in certain disease states of the hematopoietic system, dysregulation of cellular regulatory pathways may lead to progenitor cells that acquire the ability to self-renew. For instance, acute myeloid leukemia (AML, also called acute myelogenous leukemia) is a myeloproliferative disorder marked, in part, by infiltration of bone marrow by abnormal hematopoietic cells. AML is categorized into different subtypes based on morphological features and cytochemical staining properties, and although the self-renewal characteristic in most types of AML is attributable to leukemic cells having cell marker phenotypes consistent with HSCs (Bonnet, D. and Dick, J. E., Nat. Med. 3(7):730-737 (1997)), the chromosomal abnormality associated with the AML M3 subtype is observed in cell populations with a cell marker phenotype characteristic of more differentiated cells of the myeloid lineage (CD34⁻, CD38⁺) whereas the HSC population in M3 does not carry the translocation (Turhan, A. G. et al., Blood 79:2154-2161 (1995)).

Gain of self-renewing characteristic in the committed progenitor cell population is also suggested in chronic myeloid leukemia (CML, also called chronic myelogenous leukemia, or chronic granulocytic leukemia), a disease commonly associated with the Philadelphia chromosome, which is a balanced translocation between chromosomes 9 and 22, t(9;22). The translocation produces a fusion between the bcr and c-abl genes and results in expression of a chimeric protein BCR-ABL with increased tyrosine kinase activity. Although the HSC population in CML typically contains the chromosomal abnormality, the BCR-ABL fusion protein is mainly expressed in the committed cells of myelomonocytic lineage rather than the HSCs, indicating that committed cells in the myeloid lineage may be the source of the leukemic cells rather than the HSCs. Additional evidence for the committed myeloid cells as being the source of the leukemic clones in CML comes from studies of controlled expression of BCR-ABL in transgenic animals. Use of promoters active specifically in myeloid progenitor cells to force expression of BCR-ABL in committed cells but not in HSCs produces disease characteristic of CML in these transgenic animal models (Jaiswal, S. et al., Proc. Natl. Acad. Sci. USA 100:10002-10007 (2003)).

Although myeloproliferative disorders, such as AML and CML are typically associated with cytogenetic abnormalities, the cytogenetic defect may not be solely responsible for the proliferative trait. In some instances, the chromosomal abnormality is observed in normal cells, which suggests that accumulation of additional mutations in either the HSCs or committed myeloid cells is required for full manifestation of the disease state. Even in CML, the disorder displays a multiphasic course, beginning from a chronic phase, which after 3-5 years and up to 10 years, leads to an accelerated or blastic phase similar to AML. The time period required to transition from the chronic phase (less than 5% blasts or promyelocytes) to the blastic phase (>30% blasts in the peripheral blood or bone marrow) may reflect the time needed to accumulate the mutations responsible for conversion of the chronic phase to the more aggressive blastic phase. For the most part, however, the leukemic cells appear to retain the cell marker phenotypes detectable in normal progenitor cells.

The origin of leukemic stem cells from committed progenitor cells is also indicated for proliferative diseases of the lymphoid lineage. Analysis of patients with B-cell chronic lymphocytic leukemia for expression of cell marker CD38 shows that those with higher percentages of CD38 positive cells had poor clinical outcomes and showed increased refractoriness to chemotherapy (Durig, J. et al., Leukemia 16(1):30-35 (2002)). Since human HSCs are CD38⁻, the data may indicate that certain lymphoid malignancies arise from lymphoid progenitor cells rather than HSCs. Leukemic cells having a lymphoid progenitor cell marker phenotype appears also in acute lymphoblastic leukemia (ALL) (Hotfilder, M. et al., Blood 100(2):640-646 (2002); Suzuki, S. et al., Leuk. Lymphoma 44(5):849-57 (2003)).

Treatments for proliferative disorders normally rely on the sensitivity of proliferating cells to cytotoxic or cytostatic chemotherapeutic agents. For instance, busulfan, a bifunctional alkylating agent, and hydroxyurea, an inhibitor of ribonucleoside diphosphate, affect DNA synthesis and stability, resulting in toxicity to dividing cells. Other therapeutic agents of similar activity include cytosine arabinoside (cytarabine) and daunorubicin. However, the effects of these agents are non-discriminatory and as a result they have serious side effects due to toxicity to normal dividing cells.

Another treatment used in patients with haematological malignancies is bone marrow transplant (BMT), where the recipient's hematopoietic cells are eliminated with radiation and/or chemotherapy (e.g., cyclophosphamide), and the hematopoietic system reconstituted by transplant of healthy hematopoietic stem cells. Typically, the transplant uses HLA matched allogeneic bone marrow cells from a family member (HLA-identical) or a serologically matched altruistic donor (MUD). Approximately, <50% of recipients find a donor, with exactly matching histocompatibility. Transplant with less well matched donors marketed increases the transplant related morbidity and mortality. This therapeutic approach has limited application because of its dependence on the availability of suitable donors and because the treatments show better outcome for patients in the chronic or early phase of the disease as compared to acute or late stages.

Antibody therapy for cancer involves the use of antibodies, or antibody fragments, against an antigen to target antigen-expressing tumor cells. Because antibody therapy targets cells expressing a particular antigen, there is a possibility of cross-reactivity with normal cells and can lead to detrimental results. Substantial efforts have been directed to finding tumor-specific antigens. Tumor-specific antigens are found almost exclusively on tumors or are expressed at a greater level in tumor cells than the corresponding normal cells. Thus, tumor-specific antigens provide targets for antibody targeting of cancer, or other disease-related cells, expressing the antigen. Antibodies specific to such tumor-specific antigens can be conjugated to cytotoxic compounds or can be used alone in immunotherapy.

Immunotherapy as a treatment option against hematpoietic cancers, such as AML, is limited by the lack of tumor-associated antigens that are tumor-specific and that are shared among diverse patients. It is desirable to find other therapeutic agents that take advantage of the developmental origins of the leukemic cells by exploiting the common characteristics between leukemic cells and normal cell populations in the myeloid or lymphoid lineage. This approach would provide treatments that can supplement traditional therapies or that can be used as an alternative treatment to directly target the leukemic cells based on their developmental origin.

The CD45 family of transmembrane protein tyrosine phosphatases plays a critical role in lymphocyte activation by regulating the phosphorylation and activity of src-family protein tyrosine kinases and their substrates. (See Basadonna, G. P. et al. (1998) Proc. Natl. Acad. Sci., USA Vol. 95, pp. 3821-3826 incorporated herein by reference).

### 3. SUMMARY

The present invention provides antibodies effective in the diagnosis and treatment of human hematopoietic cancers. As described herein, CD45RA, an exon 4 splice variant of the CD45 family, has been found to be associated with hematopoietic tumor cells (HTCs). Accordingly, compositions including antibodies that specifically bind the CD45RA antigen and methods of using the same are provided herein to treat haematological proliferative disorders characterized by CD45RA+ hematopoietic tumor cells (HTCs). The antibodies provided by the present invention specifically bind to CD45RA on HTCs and, as shown herein, inhibit their proliferation and/or mediate their destruction. Advantageously, the antibodies demonstrate no or minimal immunoreactivity with hematopoietic stem cells. The invention further provides immortal cell lines that produce such antibodies.

In one aspect, the invention provides antibodies that specifically bind to CD45RA on HTCs and thereby inhibit their proliferation and/or mediate their destruction. In one embodiment, the invention provides a monoclonal antibody designated 17.1 which is produced from the hybridoma cell line designated 17.1 and deposited under American Type Culture Collection (ATCC) Accession No. PTA-7339. As demonstrated herein, the 17.1 mAb specifically binds to CD45RA+ hematopoietic tumor cells including, without limitation, chronic myeloid leukemia (CML) blasts, acute myeloid leukemia (AML) blasts, as well as to cells from the KG-1a, Pfeiffer, MOLT-3, GA-10, Ramos, and Jurkat cell lines, but demonstrates minimal or no immunoreactivity with human stem cells. Surprisingly, as further demonstrated herein, binding of the 17.1 mAb to its target antigen induces apoptosis in hematopoietic tumor cell lines and patient specimens of AML and CML but does not induce apoptosis in normal NK, T and B cells, and hence offers significant therapeutic opportunities.

In another embodiment, the present invention provides antibodies that specifically bind to the epitope that is specifically bound by the monoclonal antibody produced by hybridoma cell line 17.1. In further embodiments, the present invention provides antibodies that correspond to the monoclonal antibody produced by hybridoma cell line 17.1. In preferred embodiments, the anti-CD45RA antibodies of the present invention specifically bind to SEQ ID NO:1 or a portion thereof.

In preferred embodiments, the invention provides antibodies that specifically bind to CD45RA on HTCs and thereby inhibit their proliferation and/or mediate their destruction. In particularly preferred embodiments, the anti-CD45RA antibodies of the invention induce apoptosis of a CD45RA+ hematopoietic tumor cell (HTC) such as, e.g., an AML blast crisis cell, a CML blast crisis cell, a KG1a cell, a MOLT-3 cell, a Pfeiffer cell, a Ramos cell, a GA-10 cell, a Kasumi-4 cell, and a Jurkat cell, but still more preferably do not induce apoptosis of the normal hematopoietic cells to which they bind.

In addition, the present invention provides antibodies that are specifically immunoreactive with CD45RA+ HTCs but are minimally crossreactive or not crossreactive with HSCs. In a preferred embodiment, the anti-CD45RA antibody is a monoclonal antibody. In some embodiments, the antibody is an IgG isotype or a humanized antibody. In one embodiment, the humanized antibody is from a transgenic animal that includes a human immunoglobulin gene.

In another embodiment, the invention provides an anti-CD45RA antibody complex having at least one antibody that specifically binds to CD45RA on HTCs. In a preferred embodiment, the antibody complex comprises a multimer comprising the monoclonal antibody produced by hybridoma cell line 17.1. In an alternative embodiment, the complex comprises an antibody that corresponds to the monoclonal antibody produced by hybridoma cell line 17.1. In still further embodiment, the antibody complex comprises an anti-CD45RA antibody such as MEM-56.

In alternative embodiments, the antibodies of the present invention include detectable moieties, radioactive compounds (e.g. radioisotopes or radionuclides), or bioactive compounds (e.g. drugs or small molecules). In some embodiments, the bioactive compound is a cytotoxic agent.

The subject anti-CD45RA antibodies have significant therapeutic and diagnostic utilities and in additional aspects pharmaceutical compositions, methods and kits are provided employing the subject antibodies for use in diagnosing and treating haematological proliferative disorders characterized by the presence of CD45RA+ HTCs such as, e.g., acute myelogenous leukemia, acute myelomonocytic leukemia, acute lymphoblastic leukemia, acute lymphocytic leukemia, chronic lymphocytic leukemia, chronic myelogenous leukemia, B cell large cell lymphoma, malignant lymphoma, lymphosarcoma cell leukemia, B-cell lymphoma, T-cell lymphoma, acute myeloid leukemia, non-Hodgkin lymphoma, and Hodgkin's disease.

In one aspect, the present disclosure provides methods of using anti-CD45RA antibodies to target CD45RA+ hematopoietic tumor cells (HTCs). In the present teachings, the antibodies provide a basis for therapeutic approaches in treating disorders involving CD45RA+ hematopoietic tumor cells (HTCs) of the hematopoietic system, for example, myeloproliferative disorders such as chronic myeloid leukemia (CML) and acute myeloid leukemia (AML) and lymphoid proliferative disorders such as acute lymphocytic leukemia (ALL) and non-Hodgkin lymphoma (NHL).

In one embodiment, the present invention provides methods of inhibiting the proliferation of CD45RA+ HTCs by contacting the HTCs with a composition comprising an anti-CD45RA antibody. In another embodiment, the present invention provides methods of mediating the destruction of CD45RA+ HTCs by contacting the HTCs with a composition comprising an anti-CD45RA antibody. In a preferred embodiment, the invention provides methods for inducing apoptosis in CD45RA+ HTCs by contacting the HTCs with a composition comprising an anti-CD45RA antibody. In one embodiment, the antibody is a monoclonal antibody that specifically binds an epitope on CD45RA corresponding to SEQ ID NO:1 or a portion thereof. In another embodiment, the composition comprises an anti-CD45RA antibody complex.

In another embodiment, a method of depleting CD45RA+ HTCs in a subject in need thereof is provided in which the subject is administered a composition comprising an anti-CD45RA antibody or antibody complex as described herein. In yet another embodiment, the present invention provides a method of treating a patient with a haematological proliferative disorder characterized by CD45RA+ HTCs where the patient is administered a composition that includes an anti-CD45RA antibody or antibody complex as described herein.

In some embodiments, the methods of the present invention are suitable for treating a haematological proliferative disorder including myoproliferative disorders and/or lymphoproliferative disorders. The present invention also provides methods of treating a myoproliferative disorder that is chronic myeloid leukemia (CML) and/or acute myeloid leukemia (AML). In addition, the methods of treating a lymphoproliferative disorder include treating acute lymphocytic leukemia (ALL) and/or non-Hodgkin lymphoma (NHL).

### 3. BRIEF DESCRIPTION OF THE DRAWINGS

FIG. 1A-E show FACS analysis of the antibody produced by the hybridoma cell line 17.1.

FIG. 2 shows antibody 17.1 immunoreactivity to Jurkat cells.

FIG. 3 shows antibody 17.1 immunoreactivity to PBMC cells.

FIG. 4 shows antibody 17.1 immunoreactivity to CML blasts.

FIG. 5 shows antibody 17.1 immunoreactivity to CML primary cells.

FIG. 6 shows antibody 17.1 immunoreactivity to AML primary cells.

FIGS. 7A-C show immunoprecipitation of KG1a cell lysates with antibodies 17.1 (A), Western Blot analysis of KG1a cells with mAb 17.1 (B), and epitope mapping of mAb 17.1 (C).

FIGS. 8A-B show induction of apoptosis with antibody 17.1 in Kasumi-4 cells at 4 (A) and 8 (B) hrs.

FIG. 9 shows the induction of apoptosis with antibody 17.1 at 1, 5,10, and 20 μg in a CML patient sample at 4 hrs.

FIG. 10 shows no induction of ADCC with 17.1 mAb in Kasumi-4 cells.

FIG. 11 shows no induction of CDC with 17.1 mAb in Kasumi-4 cells.

FIG. 12A shows the CML peripheral blood sample-blast crisis. FIG. 12B shows the binding of mAb 17.1 to CML peripheral blood sample.

FIG. 13 shows NOD/SCID analysis of CD34 compartment, bone marrow, 11 weeks post transplant.

FIG. 14 shows CML cells CD34⁺CD45RA⁺ sorted from the bone marrow and spleen of several mice for secondary transplantation.

FIGS. 15A-B shows the NOD/SCID analysis, secondary transplant, 10 weeks post transplant with the CD34 compartment of bone marrow (A) and spleen (B).

FIGS. 16A-B shows a sample of CML peripheral blood sample-blast crisis, patient sample (A) and binding of mAb 17.1 to CML peripheral blood sample (B).

FIG. 17 is NOD/SCID analysis of CD34 compartment, bone marrow, 8 weeks post transplant.

FIG. 18 shows apoptosis of KG1a cells incubated with antibody 17.1 and antibody MEM-56, a monoclonal to human CD45RA, with and without crosslinkers.

FIG. 19 shows the degree to which the 17.1 antibody can induce apoptosis in the RL cell line at 0.1, 1, 5, and 7 μg.

FIG. 20 shows the titration of the 17.1 monoclonal antibody in an apoptotic assay as the percentage of apoptosis at various concentrations of the antibody.

FIG. 21 shows the degree to which the 17.1 antibody can induce apoptosis in the MOLT-3 cell line at 0.1, 1, 5, and 7 μg.

FIG. 22 shows the results of an MTT assay of Pfeiffer cells treated with the 17.1 antibody.

FIG. 23 shows the results of a cell count performed following incubation of the 17.1 antibody with Pfeiffer cells.

FIG. 24 shows the degree to which the 17.1 antibody inhibits the growth of Pfeiffer cells in multi-well plates at various concentrations (3 ug, 10 ug, 20 ug, and 30 ug)

FIG. 25 shows confocal microscopy results on the internalization of the 17.1 antibody upon binding to cell surface receptors.

FIG. 26 illustrates the expression of CD45RA on AML patient UOV12-38 blood sample by FACS.

FIG. 27 shows analysis of a NOD/SCID mouse transplanted with an AML patient sample UOV12-38.

FIG. 28 shows expression of CD45RA by the cell line KG-1a.

FIG. 29 shows expression of CD45RA by the cell line GA-10.

FIG. 30 shows expression of CD45RA by the cell line Ramos.

FIG. 31 demonstrates that upon subcutaneous transplantation of KG-1a cells in mice, the KG-1a cells maintain expression of CD45RA.

FIG. 32 shows a FACs histogram demonstrating that none of the commercial antibody fully inhibits the binding capability of CSC17.1.

### 4. DETAILED DESCRIPTION OF EMBODIMENTS

### 4.1 Definitions

For the following descriptions, the technical and scientific terms used herein will have the meanings commonly understood by one of ordinary skill in the art, unless specifically defined otherwise. Accordingly, the following terms are intended to have the following meanings:

“Antibody” refers to a composition comprising a protein that binds specifically to a corresponding antigen and has a common, general structure of immunoglobulins. The term antibody specifically covers polyclonal antibodies, monoclonal antibodies, dimers, multimers, multispecific antibodies (e.g., bispecific antibodies), and antibody fragments, so long as they exhibit the desired biological activity. Antibodies may be murine, human, humanized, chimeric, or derived from other species. Typically, an antibody will comprise at least two heavy chains and two light chains interconnected by disulfide bonds, which when combined form a binding domain that interacts with an antigen. Each heavy chain is comprised of a heavy chain variable region (V_{H}) and a heavy chain constant region (C_{H}). The heavy chain constant region is comprised of three domains, C_{H1}, C_{H2} and C_{H3}, and may be of the mu, delta, gamma, alpha or epsilon isotype. Similarly, the light chain is comprised of a light chain variable region (V_{L}) and a light chain constant region (C_{L}). The light chain constant region is comprised of one domain, C_{L}, which may be of the kappa or lambda isotype. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The heavy chain constant region mediates binding of the immunoglobulin to host tissue or host factors, particularly through cellular receptors such as the Fc receptors (e.g., FcγRI, FcγRII, FcγRIII, etc.). As used herein, antibody also include an antigen binding portion of an immunoglobulin that retains the ability to bind antigen. These include, as examples, F(ab), a monovalent fragment of V_{L} C_{L} and V_{H} C_{H} antibody domains; and F(ab)₂ fragment, a bivalent fragment comprising two Fab fragments linked by a disulfide bridge at the hinge region. The term antibody also refers to recombinant single chain Fv fragments (scFv) and bispecific molecules such as, e.g., diabodies, triabodies, and tetrabodies (see, e.g., U.S. Pat. No. 5,844,094).

Antibodies may be produced and used in many forms, including antibody complexes. As used herein, the term “antibody complex” or “antibody complexes” is used to mean a complex of one or more antibodies with another antibody or with an antibody fragment or fragments, or a complex of two or more antibody fragments. Antibody complexes include multimeric forms of anti-CD45RA antibodies such as homoconjugates and heteroconjugates as well as other cross-linked antibodies as described herein.

“Antigen” is to be construed broadly and refers to any molecule, composition, or particle that can bind specifically to an antibody. An antigen has one or more epitopes that interact with the antibody, although it does not necessarily induce production of that antibody.

The terms “cross-linked”, “cross-linking” and grammatical equivalents thereof, refer to the attachment of two or more antibodies to form antibody complexes, and may also be referred to as multimerization. Cross-linking or multimerization includes the attachment of two or more of the same antibodies (e.g. homodimerization), as well as the attachment of two or more different antibodies (e.g. heterodimerization). Those of skill in the art will also recognize that cross-linking or multimerization is also referred to as forming antibody homoconjugates and antibody heteroconjugates. Such conjugates may involve the attachment of two or more monoclonal antibodies of the same clonal origin (homoconjugates) or the attachment of two or more antibodies of different clonal origin (also referred to as heteroconjugates or bispecific). Antibodies may be crosslinked by non-covalent or covalent attachment. Numerous techniques suitable for cross-linking will be appreciated by those of skill in the art. Non-covalent attachment may be achieved through the use of a secondary antibody that is specific to the primary antibody species. For example, a goat anti-mouse (GAM) secondary antibody may be used to cross-link a mouse monoclonal antibody. Covalent attachment may be achieved through the use of chemical cross-linkers.

“Epitope” refers to a determinant capable of specific binding to an antibody. Epitopes are chemical features generally present on surfaces of molecules and accessible to interaction with an antibody. Typical chemical features are amino acids and sugar moieties, having three-dimensional structural characteristics as well as chemical properties including charge, hydrophilicity, and lipophilicity. Conformational epitopes are distinguished from non-conformational epitopes by loss of reactivity with an antibody following a change in the spatial elements of the molecule without any change in the underlying chemical structure.

“Humanized antibody” refers to an immunoglobulin molecule containing a minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. A humanized antibody will also encompass immunoglobulins comprising at least a portion of an immunoglobulin constant region (Fc), generally that of a human immunoglobulin (Jones et al., Nature 321:522-525 (1986); Reichmann et al, Nature 332:323-329 (1988)).

“Immunogen” refers to a substance, compound, or composition which stimulates the production of an immune response.

The term “immunoglobulin locus” refers to a genetic element or set of linked genetic elements that comprise information that can be used by a B cell or B cell precursor to express an immunoglobulin peptide. This peptide can be a heavy chain peptide, a light chain peptide, or the fusion of a heavy and a light chain peptide. In the case of an unrearranged locus, the genetic elements are assembled by a B cell precursor to form the gene encoding an immunoglobulin peptide. In the case of a rearranged locus, a gene encoding an immunoglobulin peptide is contained within the locus.

Isotype” refers to an antibody class defined by its heavy chain constant region. Heavy chains are generally classified as gamma, mu, alpha, delta, epsilon and designated as IgG, IgM, IgA, IgD, and IgE. Variations within each isotype are categorized into subtypes, for example subtypes of IgG are divided into IgG₁, IgG₂, IgG₃, and IgG₄, while IgA is divided into IgA₁ and IgA₂. The IgY isotype is specific to birds.

“Monoclonal antibody” or “monoclonal antibody composition” refers to a preparation of antibody molecules of single molecular composition. A monoclonal antibody composition displays a single binding specificity and affinity for a particular epitope.

The term “human monoclonal antibody” refers to antibodies displaying a single binding specificity which have variable and/or constant regions (if present) derived from human germline immunoglobulin sequences. In one embodiment, the human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

“Haematological proliferative disorder” or “hematopoietic proliferative disorder” refers to a condition characterized by the clonal proliferation of one or more hematopoietic cells of the myeloid and/or lymphoid lineage. Such proliferative disorders include, the general classes of (a) dysmyelopoietic disease, (b) acute myeloproliferative leukemia, (c) chronic myeloproliferative disease, (d) acute lymphoproliferative leukemia, (e) Hodgkin lymphomas, and (f) non-Hodgkin lymphomas. Each general class is further categorized into different subtypes, as is known in the art.

“Single chain Fv” or “scFv” refers to an antibody comprising the V_{H} and V_{L} regions of an antibody, wherein these domains are present in a single polypeptide chain. Generally, an scFv further comprises a polypeptide linker between the V_{H} and V_{L} domains which enables the scFv to form the desired structure for antigen binding.

“Specifically immunoreactive” or “antibody that specifically binds to” refers to a binding reaction of the antibody that is determinitive of the presence of the antigen in a heterogeneous population of antigens. Under a designated immunoassay condition, the antibody binds to the antigen at least two times, and typically 10-1000 times or more over background. “Specifically immunoreactive” or “antibody that specifically binds” also refers to an antibody that is capable of binding to an antigen with sufficient affinity such that the antibody is useful in targeting a cell expressing the antigen. In such embodiments, the extent of non-specific binding is the amount of binding at or below background and will typically be less than about 10%, preferably less than about 5%, and more preferably less than about 1% as determined by fluorescence activated cell sorting (FACS) analysis or radioimmunoprecipitation (RIA), for example.

“Subject” or “patient” are used interchangeably and refer to, except where indicated, mammals such as humans and non-human primates, as well as rabbits, rats, mice, goats, pigs, and other mammalian species.

“Recombinant antibody” refers to all antibodies prepared and expressed, created or isolated by recombinant techniques. These include antibodies obtained from an animal that is transgenic for the immunoglobulin locus, antibodies expressed from a recombinant expression vector, or antibodies created, prepared, and expressed by splicing of any immunoglobulin gene sequence to other nucleic acid sequences.

### 4.2 Hybridomas and Monoclonal Antibodies

The teachings of the present disclosure provide hybridoma cell lines and monoclonal antibodies that specifically bind to CD45RA+ hematopoietic tumor cells (HTCs). Provided herein is a hybridoma cell line designated 17.1. Hybridoma 17.1, secreting a monoclonal antibody designated 17.1 (mAb 17.1), was deposited on Jan. 31, 2006, with the American Type Culture Collection (ATCC), Patent Depository, 10801 University Blvd. Manassas, Va. 20110, under the provisions of the Budapest Treaty on the International Recognition of the Deposit of Microorganisms for the Purpose of Patent Procedures, and assigned accession number PTA-7339.

The present disclosure further provides a method of producing mAb 17.1 or derivatives thereof comprising: cultivating a 17.1 hybridoma cell under suitable conditions, wherein a 17.1 antibody is produced and obtaining the antibody and/or derivative thereof from the cell and/or from the cell culture medium.

The present disclosure further provides the mAb 17.1 and derivatives thereof. Monoclonal antibody 17.1 is specifically immunoreactive with cells of the hematopoietic system. The mAb 17.1 is specifically immunoreactive with granulocyte/macrophage progenitors (GMP), KG-1a, K-562, Jurkat, CML blasts, and AML blasts. The mAb 17.1 shows no immunoreactivity with human stem cells (HSCs), common myeloid progenitors (CMP), and PBMC cells. Notably, as demonstrated herein, mAb 17.1 induces apoptosis in hematopoietic tumor cells but not in the normal hematopoietic cells to which it binds.

The present disclosure also encompasses any antibody that recognizes the antigen recognized by the antibody produced by hybridoma 17.1. As demonstrated herein, mAb 17.1 specifically binds to the CD45RA antigen and more particularly to the epitope of CD45RA defined by SEQ ID NO:1. In one embodiment, therefore, the subject anti-CD45RA antibodies specifically bind to all or a portion of SEQ ID NO:1. Antibodies that specifically bind all or a portion of SEQ ID NO:1 may be readily identified by competitive inhibition assays and the like using mAb 17.1. In another embodiment, the subject anti-CD45RA antibodies specifically bind to an epitope of CD45RA corresponding to SEQ ID NO:1 having at least 80%, more preferably 85%, more preferably 90%, more preferably 95% sequence identity to SEQ ID NO: 1.

In another preferred embodiment, the present invention contemplates antibodies that correspond to the monoclonal antibody produced by hybridoma 17.1. One antibody corresponds to another antibody if they both recognize the same or overlapping antigen binding sites as demonstrated by, for example, a binding inhibition assay.

Antibodies can be produced readily by one skilled in the art. The general methodology for making monoclonal antibodies by hybridomas is now well known to the art. See, e.g., M. Schreier et al., Hybridoma Techniques (Cold Spring Harbor Laboratory); Hammerling et al., Monoclonal Antibodies and T-Cell Hybridomas (Elsevier Biomedical Press). In a further embodiment, monoclonal antibodies described above may be obtained using the antigen that is specifically immunoreactive with mAb 17.1 directly as an immunogen. The monoclonal antibody produced by hybridoma 17.1 can be readily employed to precipitate its antigen, as demonstrated herein. For example, the antigen that is specifically immunoreactive with mAb 17.1 can be immunoprecipitated from cell extracts of the GMP, KG-1a, Jurkat, CML primary cells, AML primary cells or other cells. The precipitated antigen can then be used as an immunogen. By application of any of the above methods, one skilled in the art can readily produce an antibody specific to the antigen recognized by mAb 17.1.

As described above, the present disclosure provides methods of producing the monoclonal antibodies or derivatives thereof. In some embodiments, these methods comprise cultivating a hybridoma cell under suitable conditions, wherein the antibody is produced and obtaining the antibody and/or derivative thereof from the cell and/or from the cell culture medium.

The antibodies can be purified by methods known to the skilled artisan. Purification methods include, among other, selective precipitation, liquid chromatography, HPLC, electrophoresis, chromatofocusing, and various affinity techniques. Selective precipitation may use ammonium sulfate, ethanol (Cohn precipitation), polyethylene glycol, or others available in the art. Liquid chromatography mediums, include, among others, ion exchange medium DEAE, polyaspartate), hydroxylapatite, size exclusion (e.g., those based on crosslinked agarose, acrylamide, dextran, etc.), hydrophobic matrixes (e.g., Blue Sepharose). Affinity techniques typically rely on proteins that interact with the immunoglobulin Fc domain. Protein A from Staphylococcus aureas can be used to purify antibodies that are based on human γ1, γ2, or γ4 heavy chains (Lindmark et al., J. Immunol. Meth. 62:1-13 (1983)). Protein G from C and G streptococci is useful for all mouse isotypes and for human .γ3 (Guss et al., EMBO J. 5:15671575 (1986)). Protein L, a Peptostreptococcus magnus cell-wall protein that binds immunoglobulins (Ig) through k light-chain interactions (BD Bioscience/ClonTech. Palo Alto, Calif.), is useful for affinity purification of Ig subclasses IgM, IgA, IgD, IgG, IgE and IgY. Recombinant forms of these proteins are also commercially available. If the antibody contains metal binding residues, such as phage display antibodies constructed to contain histidine tags, metal affinity chromatography may be used. When sufficient amounts of specific cell populations are available, antigen affinity matrices may be made with the cells to provide an affinity method for purifying the antibodies.

The present invention provides the antibodies described herein, as well as corresponding antibody fragments and antigen-binding portions. The terms “antibody fragment” or “antigen-binding portion” of an antibody (or simply “antibody portion”) of the present invention, as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antibody fragment” or “antigen-binding portion” of an antibody include (i) a Fab fragment, a monovalent fragment consisting of the V_{L}, V_{H}, C_{L} and C_{H1}, domains; (ii) a F(ab′)₂ fragment, a bivalent fragment comprising two Fab fragments linked by a disulfide bridge at the hinge region; (iii) a Fd fragment consisting of the V_{H} and C_{H1}, domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR), and (vii) bispecific single chain Fv dimers (PCT/US92/09965). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. The antibody fragments may be modified. For example, the molecules may be stabilized by the incorporation of disulphide bridges linking the V_{H} and V_{L} domains (Reiter et al., 1996, Nature Biotech. 14:1239-1245).

The present disclosure further provides fragments of the antibodies disclosed herein. Immunoglobulin molecules can be cleaved into fragments. The antigen binding region of the molecule can be divided into either F(ab′)₂ or Fab fragments. The F(ab′)₂ fragment is divalent and is useful when the Fc region is either undesirable or not a required feature. The Fab fragment is univalent and is useful when an antibody has a very high avidity for its antigen. Eliminating the Fc region from the antibody decreases non-specific binding between the Fc region and Fc receptor bearing cells. To generate Fab or F(ab)₂ fragments, the antibodies are digested with an enzyme. Proteases that cleave at the hinge region of an immunoglobulin molecule preserve the disulfide bond(s) linking the F(ab) domain such that they remain together following cleavage. A suitable protease for this purpose is pepsin. For producing F(ab) fragments, proteases are chosen such that cleavage occurs above the hinge region containing the disulfide bonds that join the heavy chains but which leaves intact the disulfide bond linking the heavy and light chain. A suitable protease for making F(ab) fragments is papain. The fragments are purified by the methods described above, with the exception of affinity techniques requiring the intact Fc region (e.g., Protein A affinity chromatography).

Antibody fragments can be produced by limited proteolysis of antibodies and are called proteolytic antibody fragments. These include, but are not limited to, the following: F(ab′)₂ fragments, Fab′ fragments, Fab′-SH fragments, and Fab fragments. “F(ab′)₂ fragments” are released from an antibody by limited exposure of the antibody to a proteolytic enzyme, e.g., pepsin or ficin. An F(ab′)₂ fragment comprises two “arms,” each of which comprises a variable region that is directed to and specifically binds a common antigen. The two Fab′ molecules are joined by interchain disulfide bonds in the hinge regions of the heavy chains; the Fab′ molecules may be directed toward the same (bivalent) or different (bispecific) epitopes. “Fab′ fragments” contain a single anti-binding domain comprising an Fab and an additional portion of the heavy chain through the hinge region. “Fab′-SH fragments” are typically produced from F(ab′)₂ fragments, which are held together by disulfide bond(s) between the H chains in an F(ab′)₂ fragment. Treatment with a mild reducing agent such as, by way of non-limiting example, beta-mercaptoethylamine, breaks the disulfide bond(s), and two Fab′ fragments are released from one F(ab′)₂ fragment. Fab′-SH fragments are monovalent and monospecific. “Fab fragments” (i.e., an antibody fragment that contains the antigen-binding domain and comprises a light chain and part of a heavy chain bridged by a disulfide bond) are produced by papain digestion of intact antibodies. A convenient method is to use papain immobilized on a resin so that the enzyme can be easily removed and the digestion terminated. Fab fragments do not have the disulfide bond(s) between the H chains present in an F(ab′)₂ fragment.

“Single-chain antibodies” are one type of antibody fragment. The term single chain antibody is often abbreviated as “scFv” or “sFv.” These antibody fragments are produced using molecular genetics and recombinant DNA technology. A single-chain antibody consists of a polypeptide chain that comprises both a V.sub.H and a V.sub.L domains which interact to form an antigen-binding site. The V.sub.H and V.sub.L domains are usually linked by a peptide of 10 to 25 amino acid residues.

The term “single-chain antibody” further includes but is not limited to a disulfide-linked Fv (dsFv) in which two single-chain antibodies (each of which may be directed to a different epitope) linked together by a disulfide bond; a bispecific sFv in which two discrete scFvs of different specificity is connected with a peptide linker; a diabody (a dimerized sFv formed when the V.sub.H domain of a first sFv assembles with the V.sub.L domain of a second sFv and the V.sub.L domain of the first sFv assembles with the V.sub.H domain of the second sFv; the two antigen-binding regions of the diabody may be directed towards the same or different epitopes); and a triabody (a trimerized sFv, formed in a manner similar to a diabody, but in which three antigen-binding domains are created in a single complex; the three antigen binding domains may be directed towards the same or different epitopes).

“Complementary determining region peptides” or “CDR peptides” are another form of an antibody fragment. A CDR peptide (also known as “minimal recognition unit”) is a peptide corresponding to a single complementarity-determining region (CDR), and can be prepared by constructing genes encoding the CDR of an antibody of interest. Such genes are prepared, for example, by using the polymerase chain reaction to synthesize the variable region from RNA of antibody-producing cells. See, for example, Larrick et al., Methods: A Companion to Methods in Enzymology 2:106, 1991.

In “cysteine-modified antibodies,” a cysteine amino acid is inserted or substituted on the surface of antibody by genetic manipulation and used to conjugate the antibody to another molecule via, e.g., a disulfide bridge. Cysteine substitutions or insertions for antibodies have been described (see U.S. Pat. No. 5,219,996). Methods for introducing Cys residues into the constant region of the IgG antibodies for use in site-specific conjugation of antibodies are described by Stimmel et al. (J. Biol. Chem 275:330445-30450, 2000).

The present disclosure further provides humanized and non-humanized antibodies. Humanized forms of non-human (e.g., mouse) antibodies are chimeric antibodies that contain minimal sequence derived from non-human immunoglobulin. Generally, humanized antibodies are non-human antibodies that have had the variable-domain framework regions swapped for sequences found in human antibodies. The humanized antibodies may be human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin.

Generally, in a humanized antibody, the entire antibody, except the CDRs, is encoded by a polynucleotide of human origin or is identical to such an antibody except within its CDRs. The CDRs, some or all of which are encoded by nucleic acids originating in a non-human organism, are grafted into the beta-sheet framework of a human antibody variable region to create an antibody, the specificity of which is determined by the engrafted CDRs. The creation of such antibodies is described in, e.g., WO 92/11018, Jones, 1986, Nature 321:522-525, Verhoeyen et al., 1988, Science 239:1534-1536. Humanized antibodies can also be generated using mice with a genetically engineered immune system. Roque et al., 2004, Biotechnol. Prog. 20:639-654.

The present disclosure further provides humanized and non-humanized antibodies. Humanized forms of non-human (e.g., mouse) antibodies are chimeric antibodies that contain minimal sequence derived from non-human immunoglobulin. Generally, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin.

It can be desirable to modify the antibodies of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176:1191-1195 (1992) and Shopes, J. Immunol., 148:2918-2922 (1992). Homodimeric antibodies with enhanced anti-tumor activity can also be prepared using heterobifunctional cross-linkers as described in Wolff et al. Cancer Research, 53:2560-2565 (1993). Alternatively, an antibody can be engineered that has dual Fc regions and can thereby have enhanced complement lysis and ADCC capabilities. See Stevenson et al., Anti-Cancer Drug Design, 3:219-230 (1989).

In some embodiments, the antibodies described herein specifically bind to a CD45RA antigen present on the cell surface of hematopoietic tumor cells (HTCs) that arose from progenitor cell populations in the myeloid and/or lymphoid lineage of the hematopoietic system. Differentiation in the myeloid or lymphoid lineage leads to formation of terminally differentiated cells that include, among others, megakaryocytes, erythroid cells, macrophages, basophils, eosinophils, neutrophils, myeloid dendritic cells, T lymphocytes, B lymphocytes, natural killer (NK) cells, and lymphoid dendritic cells. These cells originate from hematopoietic stem cells (HSC), which differentiate through a series of progenitor cell populations displaying progressively restricted differentiation potential. The HSCs and the progenitor cell populations are identifiable from each other based on, among other distinguishing characteristics, their capacity to differentiate into specific cell subsets and the presence of a particular set of cellular markers that is specific to the cell population.

In some embodiments, the monoclonal antibodies in the present disclosure are directed to committed myeloid or lymphoid progenitor cells that are CD45RA+ hematopoietic tumor cells (HTCs). Because these antibodies will have applications as therapeutics for diseases involving these committed progenitor cells, the monoclonal antibodies are preferably minimally immunoreactive with normal HSCs.

As noted above, in some embodiments, the antibodies of the present disclosure are directed to the progenitor cells of the myeloid or lymphoid lineage that are CD45RA+ hematopoietic tumor cells (HTCs). In preferred embodiments, the antibodies are only minimally crossreactive with hematopoietic stem cells (HSCs), and still more preferably are not immunoreactive with HSCs. By “minimally crossreactive” refers to less than about 25%, preferably less than about 10%, and more preferably less than about 5%, and most preferably less than about 1% of the assay signal obtained with the specifically immunoreactive cell.

4.2.1 Modified Antibodies

In another aspect, the present invention provides modified antibodies that are derived from an antibody that specifically binds CD45RA. Modified antibodies also include recombinant antibodies as described herein. In a preferred embodiment, modified antibodies are derived from a CD45RA antibody that induces apoptosis in hematopoietic tumor cells (HTCs). In another preferred embodiment, the modified antibody is derived from the monoclonal antibody 17.1. In other embodiments, the modified antibody is derived from an antibody that binds to an epitope that is specifically bound by the monoclonal antibody 17.1.

Numerous types of modified or recombinant antibodies will be appreciated by those of skill in the art. Suitable types of modified or recombinant antibodies include without limitation, engineered murine monoclonal antibodies (e.g. murine monoclonal antibodies, chimeric monoclonal antibodies, humanized monoclonal antibodies), domain antibodies (e.g. Fab, Fv, V_{H}, scFV, and dsFv fragments), multivalent or multispecific antibodies (e.g. diabodies, minibodies, miniantibodies, (scFV)₂, tribodies, and tetrabodies), and antibody conjugates as described herein.

In one aspect, the present invention includes domain antibodies. “Domain antibodies” are functional binding domains of antibodies, corresponding to the variable regions of either the heavy (VH) or light (VL) chains of human antibodies. Domain antibodies may have a molecular weight of approximately 13 kDa, or less than one-tenth the size of a full antibody. They are well expressed in a variety of hosts including bacterial, yeast, and mammalian cell systems. In addition, domain antibodies are highly stable and retain activity even after being subjected to harsh conditions, such as freeze-drying or heat denaturation. See, for example, U.S. Pat. Nos. 6,291,158; 6,582,915; 6,593,081; 6,172,197; US Serial No. 2004/0110941; European Patent 0368684; U.S. Pat. No. 6,696,245, WO04/058821, WO04/003019 and WO03/002609. In one embodiment, the domain antibody of the present invention is a single domain. Single domain antibodies may be prepared, for example, as described in U.S. Pat. No.6,248,516, incorporated herein by reference in its entirety. In some embodiments, the present invention provides domain antibodies derived from an antibody that specifically binds CD45RA. In a preferred embodiment, the present invention provides a domain antibody derived from a CD45RA antibody that induces apoptosis in hematopoietic tumor cells (HTCs). In another preferred embodiment, the domain antibody is derived from the monoclonal antibody 17.1. In other embodiments, the domain antibody is derived from an antibody that binds to an epitope that is specifically bound by the monoclonal antibody 17.1.

In another aspect, the present invention includes multi-specific antibodies. Multi-specific antibodies include bispecific, trispecific, etc. antibodies. Bispecific antibodies can be produced via recombinant means, for example by using leucine zipper moieties (i.e., from the Fos and Jun proteins, which preferentially form heterodimers; Kostelny et al., 1992, J. Immnol. 148:1547) or other lock and key interactive domain structures as described in U.S. Pat. No. 5,582,996. Additional useful techniques include those described in U.S. Pat. No. 5,959,083; and U.S. Pat. No. 5,807,706. In one embodiment, the present invention provides multi-specific antibodies that include an antibody that specifically binds CD45RA. In another embodiment, the multispecific antibody is bispecific. In a preferred embodiment, the CD45RA bispecific antibody induces apoptosis in hematopoietic tumor cells (HTCs). In another preferred embodiment, the bispecific antibody is derived from the monoclonal antibody 17.1. In other embodiments, the bispecific antibody is derived from an antibody that binds to an epitope that is specifically bound by the monoclonal antibody 17.1.

Bispecific antibodies are also sometimes referred to as “diabodies.” These are antibodies that bind to two (or more) different antigens. Also known in the art are triabodies (a trimerized sFv, formed in a manner similar to a diabody, but in which three antigen-binding domains are created in a single complex; the three antigen binding domains may be directed towards the same or different epitopes) or a tetrabodies (four antigen-binding domains created in a single complex where the four antigen binding domains may be directed towards the same or different epitopes). Dia-, tria- and tetrabodies can be manufactured in a variety of ways known in the art (Holliger and Winter, 1993, Current Opinion Biotechnol. 4:446-449), e.g., prepared chemically or from hybrid hybridomas. In addition, such antibodies and fragments thereof may be constructed by gene fusion (Tomlinson et. al., 2000, Methods Enzymol. 326:461-479; WO94/13804; Holliger et al., 1993, Proc. Natl. Acad. Sci. U.S.A. 90:6444-6448, each of which is incorporated herein by reference in their entirety). In one embodiment, the diabody, triabody, or tetrabody is derived from a CD45RA antibody. In a preferred embodiment, the diabody, triabody, or tetrabody is derived from the monoclonal antibody 17.1. In another preferred embodiment, the diabody, triabody, or tetrabody is derived from an antibody that binds to an epitope that is specifically bound by the monoclonal antibody 17.1.

In another embodiment, the present invention provides minibodies, which are minimized antibody-like proteins that include a scFV joined to a CH3 domain, that are derived from an antibody that specifically binds CD45RA. In a preferred embodiment, the minibody derived from a CD45RA antibody induces apoptosis in hematopoietic tumor cells (HTCs). In other preferred embodiments, the minibody is derived from the monoclonal antibody 17.1. In a preferred embodiment, the minibody is derived from an antibody that binds an epitope that is specifically bound by the monoclonal antibody 17.1. Minibodies can be made as described in the art (Hu et al., 1996, Cancer Res. 56:3055-3061).

In another embodiment, the present invention provides CD45RA binding domain-immunoglobulin fusion proteins. The fusion protein may include a CD45RA binding domain polypeptide fused to an immunoglobulin hinge region polypeptide, which is fused to an immunoglobulin heavy chain CH2 constant region polypeptide fused to an immunoglobulin heavy chain CH3 constant region polypeptide. Under the present invention, CD45RA antibody fusion proteins can be made by methods appreciated by those of skill in the art (See published U.S. Patent Application Nos. 20050238646, 20050202534, 20050202028, 2005020023, 2005020212, 200501866216, 20050180970, and 20050175614, each of which is incorporated herein by reference in their entirety).

In another embodiment, the present invention provides a heavy-chain protein (V.sub.HH) derived from a CD45RA antibody. Naturally-occurring heavy chain antibodies (e.g. camelidae antibodies having no light chains) have been utilized to develop antibody-derived therapeutic proteins that typically retain the structure and functional properties of naturally-occurring heavy-chain antibodies. They are known in the art as NanobodiesR. Under the present invention, heavy chain proteins (V.sub.HH) derived from a CD45RA heavy chain antibody may be made by methods appreciated by those of skill in the art (See published U.S. Patent Application Nos. 20060246477, 20060211088, 20060149041, 20060115470, and 20050214857, each of which is incorporated herein by reference in their entirety).

In one aspect, the present invention provides a modified antibody that is a human antibody. In one embodiment, the CD45RA antibodies described herein are fully human antibodies. “Fully human antibody” or “complete human antibody” refers to a human antibody having only the gene sequence of an antibody derived from a human chromosome. The anti-human CD45RA complete human antibody can be obtained by a method using a human antibody-producing mouse having a human chromosome fragment containing the genes for a heavy chain and light chain of a human antibody [see Tomizuka, K. et al., Nature Genetics, 16, p. 133-143, 1997; Kuroiwa, Y. et al., Nuc. Acids Res., 26, p. 3447-3448, 1998; Yoshida, H. et al., Animal Cell Technology: Basic and Applied Aspects vol. 10, p. 69-73 (Kitagawa, Y., Matuda, T. and Iijima, S. eds.), Kluwer Academic Publishers, 1999; Tomizuka, K. et al., Proc. Natl. Acad. Sci. USA, 97, 722-727, 2000] or obtained by a method for obtaining a human antibody derived from a phage display selected from a human antibody library (see Wormstone, I. M. et al., Investigative Ophthalmology & Visual Science. 43(7), p. 2301-8, 2002; Carmen, S. et al., Briefings in Functional Genomics and Proteomics, 1 (2), p. 189-203, 2002; Siriwardena, D. et al., Ophthalmology, 109(3), p. 427-431, 2002).

In one aspect, the present invention provides a CD45RA antibody that is an antibody analog, sometimes referred to as “synthetic antibodies.” For example, a variety of recent work utilizes either alternative protein scaffolds or artificial scaffolds with grafted CDRs. Such scaffolds include, but are not limited to, mutations introduced to stabilize the three-dimensional structure of the binding protein as well as wholly synthetic scaffolds consisting for example of biocompatible polymers. See, for example, Korndorfer et al., 2003, Proteins: Structure, Function, and Bioinformatics, Volume 53, Issue 1:121-129. Roque et al., 2004, Biotechnol. Prog. 20:639-654. In addition, peptide antibody mimetics (“PAMs) can be used, as well as work based on antibody mimetics utilizing fibronection components as a scaffold.

### 4.3 Cross-Linked Antibodies

In one aspect, the present invention provides cross-linked antibodies that include two or more antibodies described herein attached to each other to form antibody complexes. Cross-linked antibodies are also referred to as antibody multimers, homoconjugates, and heteroconjugates. It has been observed in the art that the multimerization of an antibody previously observed to have no signaling activity can result in a multimerized antibody with potent signalling activity. This has been particularly noted in the field of anti-tumor agents. For example, it has been reported that the IgG-IgG homodimerization of anti-CD19, anti-CD20, anti-CD21, anti-CD22, and anti-Her-2 monoclonal antibodies confers potent anti-tumor ability to such homodimers (Ghetie, M. et al. (1997) Proc. Natl. Acad. Sci., USA, Vol. 94, pp-7509-7514 incorporated herein by reference in its entirety). In addition, the homodimerization of monoclonal antibodies known to have anti-tumor activity, such as Rituximab®, can lead to an increase in effectiveness as an anti-tumor agent (Ghetie, M. (2001) Blood, Vo. 97;5: 1392-1398 incorporated herein by reference in its entirety).

In some embodiments, the antibody complexes provided herein include multimeric forms of anti-CD45RA antibodies. For example, antibodies complexes of the invention may take the form of antibody dimers, trimers, or higher-order multimers of monomeric immunoglobulin molecules. Crosslinking of antibodies can be done through various methods know in the art. For example, crosslinking of antibodies may be accomplished through natural aggregation of antibodies, through chemical or recombinant linking techniques or other methods known in the art. For example, purified antibody preparations can spontaneously form protein aggregates containing antibody homodimers, and other higher-order antibody multimers.

In one embodiment, the present invention provides homodimerized antibodies that specifically bind to CD45RA antigen. In a preferred embodiment, the homodimerized antibodies specifically bind to a CD45RA+ HTC and induce apoptosis of the HTC. In another preferred embodiment, the homodimerized antibody specifically binds to all or a portion of the epitope corresponding to SEQ ID NO:1. In one other specific embodiment, the homodimerized antibody is the 17.1 monoclonal antibody. In a preferred embodiment, the homodimerized antibody includes an antibody that binds to the epitope that is specifically bound by the 17.1 monoclonal antibody described herein. A non-limiting example of an anti-CD45RA antibody suitable for use in an anti-CD45RA complex includes MEM-56, as demonstrated herein.

Antibodies can be cross-linked or dimerized through linkage techniques known in the art (see Ghetie et al. (1997) supra; Ghetie et al. (2001) supra). Non-covalent methods of attachment may be utilized. In a specific embodiment, crosslinking of antibodies can be achieved through the use of a secondary crosslinker antibody. The crosslinker antibody can be derived from a different animal compared to the antibody of interest. For example, a goat anti-mouse antibody (Fab specific) may be added to a mouse monoclonal antibody to form a heterodimer. This bivalent crosslinker antibody recognizes the Fab or Fc region of the two antibodies of interest forming a homodimer.

In one embodiment of the present invention, an antibody that specifically binds to CD45RA antigen is cross-linked using a goat anti-mouse antibody (GAM). In another embodiment, the GAM crosslinker recognizes the Fab or Fc region of two antibodies, each of which specifically binds a CD45RA antigen. In a preferred embodiment, the GAM-cross-linked antibody is the 17.1 antibody and/or the MEM-56 antibody.

Methods for covalent or chemical attachment of antibodies may also be utilized. Chemical crosslinkers can be homo or heterobifunctional and will covalently bind with two antibodies forming a homodimer. Cross-linking agents are well known in the art; for example, homo-or hetero-bifunctional linkers as are well known (see the 2006 Pierce Chemical Company Crosslinking Reagents Technical Handbook; Hermanson, G. T., Bioconjugate Techniques, Academic Press, San Diego, Calif. (1996); Aslam M. and Dent A H., Bioconjugation: protein coupling techniques for the biomedical sciences, Houndsmills, England: Macmillan Publishers (1999); Pierce: Applications Handbook & Catalog, Perbio Science, Ermbodegem, Belgium (2003-2004); Haughland, R. P., Handbook of Fluorescent Probes and Research Chemicals Eugene, 9^{th} Ed., Molecular Probes, OR (2003); and U.S. Pat. No. 5,747,641; all references incorporated herein by reference) Those of skill in the art will appreciate the suitability of various functional groups on the amino acid(s) of an antibody for modification, including cross-linking. Suitable examples of chemical crosslinkers used for antibody crosslinking include, but not limited to, SMCC [succinimidyl 4-(maleimidomethyl)cyclohexane-1-carboxylate], SATA [N-succinimidyl S-acethylthio-acetate], hemi-succinate esters of N-hydroxysuccinimide; sulfo-N-hydroxy-succinimide; hydroxybenzotriazole, and p-nitrophenol; dicyclohexylcarbodiimide (DCC), 1-(3-dimethylaminopropyl)-3-ethylcarbodiimide (ECD), and 1-(3-dimethylaminopropyl)-3-ethylcarbodiimide methiodide (EDCI) (see, e.g., U.S. Pat. No. 4,526,714, the disclosure of which is fully incorporated by reference herein). Other linking reagents include glutathione, 3-(diethoxyphosphoryloxy)-1,2,3-benzotriazin-4(3H)-one (DEPBT), onium salt-based coupling reagents, polyoxyethylene-based heterobifunctional cross-linking reagents, and other reagents (Haitao, et al., Organ Left 1:91-94 (1999); Albericio et al., J Organic Chemistry 63:9678-9683 (1998); Arpicco et al., Bioconjugate Chem. 8:327-337 (1997); Frisch et al., Bioconjugate Chem. 7:180-186 (1996); Deguchi et al., Bioconjugate Chem. 10:32-37 (1998); Beyer et al., J. Med. Chem. 41:2701-2708 (1998); Drouillat et al., J. Pharm. Sci. 87:25-30 (1998); Trimble et al., Bioconjugate Chem. 8:416-423 (1997)).

Exemplary protocols for the formation of antibody homodimers is given in U.S. Patent Publication 20060062786, and Ghetie et al., (1997) supra, which are hereby incorporated by reference in their entirety. In a preferred embodiment, the chemically crosslinked antibody is the 17.1 antibody and/or the MEM-56 antibody. In a preferred embodiment, the chemical cross-linker used for the 17.1 antibody and/or the MEM-56 antibody is an SMCC or SATA crosslinker.

In addition, the antibody-antibody conjugates of this invention can be covalently bound to each other by techniques known in the art such as the use of the heterobifunctional cross-linking reagents, GMBS (maleimidobutryloxy succinimide), and SPDP (N-succinimidyl 3-(2-pyridyidithio)propionate) [see, e.g., Hardy, “Purification And Coupling Of Fluorescent Proteins For Use In Flow Cytometry”, Handbook Of Experimental Immunology, Volume 1, Immunochemistry, Weir et al. (eds.), pp. 31.4-31.12 4th Ed., (1986), and Ledbetter et al. U.S. Pat. No. 6,010,902, each of which is incorporated herein by reference in their entirety].

In addition, antibodies may be linked via a thioether cross-link as described in U.S. Patent Publication 20060216284, U.S. Pat. No. 6,368,596, which is incorporated herein by reference. As will be appreciated by those skilled in the art, antibodies can be crosslinked at the Fab region. In some embodiments, it is desirable that the chemical crosslinker not interact with the antigen-binding region of the antibody as this may affect antibody function.

### 4.4 Conjugated Antibodies

The antibodies disclosed herein can be conjugated to inorganic or organic compounds, including, by way of example and not limitation, other proteins, nucleic acids, carbohydrates, steroids, and lipids (see for example Green, et al., Cancer Treatment Reviews, 26:269-286 (2000). The compound may be bioactive. Bioactive refers to a compound having a physiological effect on the cell as compared to a cell not exposed to the compound. A physiological effect is a change in a biological process, including, by way of example and not limitation, DNA replication and repair, recombination, transcription, translation, secretion, membrane turnover, cell adhesion, signal transduction, cell death, and the like. A bioactive compound includes pharmaceutical compounds.

In one aspect, the antibodies are conjugated to or modified to carry a detectable compound. Conjugating antibodies to detectable enzymes, fluorochromes, or ligands provides a signal for visualization or quantitation of the target antigen. Antibodies may be labeled with various enzymes to provide highly specific probes that both visualize the target and amplify the signal by acting on a substrate to produce a colored or chemiluminescent product. Horseradish peroxidase, alkaline phosphatase, glucose oxidase, and □-galactosidase are the commonly used enzymes for this purpose. Fluorochromes, such as fluorescein isothiocyanate, tetramethylrhodamine isothiocyanate (TRITC), phycoerithyrin, and Cy5, provide a colored reagent for visualization and detection. Suitable fluorescent compounds are described in Haughland, R. P., Handbook of Fluorescent Probes and Research Chemicals Eugene, 9th Ed., Molecular Probes, OR (2003).

In another aspect, the conjugated compounds are chelating ligands, or macrocyclic organic chelating compounds, particularly metal chelating compounds used to image intracellular ion concentrations or used as contrast agents for medical imaging purposes. Chelating ligands are ligands that can bind with more than one donor atom to the same central metal ion. Chelators or their complexes have found applications as MRI contrast agents, radiopharmaceutical applications, and luminescent probes. Conjugates of chelating compounds useful for assessing intracellular ion concentrations may be voltage sensitive dyes and non-voltage sensitive dyes. Exemplary dye molecules for measuring intracellular ion levels include, by way of example and not limitation, Quin-2; Fluo-3; Fura-Red; Calcium Green; Calcium Orange 550 580; Calcium Crimson; Rhod-2 550 575; SPQ; SPA; MQAE; Fura-2; Mag-Fura-2; Mag-Fura-5; Di-4-ANEPPS; Di-8-ANEPPS; BCECF; SNAFL-1; SBFI; and SBFI.

In another embodiment, the ligands are chelating ligands that bind paramagnetic, superparamagnetic or ferromagnetic metals. These are useful as contrast agents for medical imaging and for delivery of radioactive metals to selected cells. Metal chelating ligands, include, by way of example and not limitation, diethylenetriaminepenta acetic acid (DTPA); diethylenetriaminepenta acetic acid bis(methylamide); macrocyclictetraamine 1,4,7,10-tetraazacyclododecane-N,N′,N″,N′″-tetraacetic acid (DOTA); and porphyrins (see, e.g., The Chemistry of Contrast Agents in Medical Magnetic Resonance Imaging, Merbach A. E. and Toth E., Ed., Wiley Interscience (2001)). Paramagnetic metal ions, which are detectable in their chelated form by magnetic resonance imaging, include, for example, iron(III), gadolinium(III), manganese (II and III), chromium(II), copper(II), dysprosium(III), terbium(III), holmium (III), erbium (III), and europium (III). Paramagnetic metal ions particularly useful as magnetic resonance imaging contrast agents comprise iron(III) and gadolinium(III) metal complexes. Other paramagnetic, superparamagnetic or ferromagnetic are well known to those skilled in the art.

In another embodiment, the metal-chelate comprises a radioactive metal. Radioactive metals may be used for diagnosis or as therapy based on delivery of small doses of radiation to a specific site in the body. Targeted metalloradiopharmaceuticals are constructed by attaching the radioactive metal ion to a metal chelating ligand, such as those used for magnetic imaging, and delivering the chelate-complex to cells. An exemplary radioactive metal chelate complex is DTPA (see, e.g., U.S. Pat. No. 6,010,679).

In a further aspect, the conjugated compounds are peptide tags used for purposes of detection, particularly through the use of antibodies directed against the peptide. Various tag polypeptides and their respective antibodies are well known in the art. Examples include poly-histidine (poly-his) or poly-histidine-glycine (poly-his-gly) tags; the flu HA tag polypeptide and its antibody 12CA5 (Field et al., Mol. Cell. Biol. 8:2159-2165 (1988)); the c-myc tag and the 8F9, 3C7, 6E10, G4, B7 and 9E10 antibodies thereto (Evan et al., Mol. Cell. Biol. 5:3610-3616 (1985)); Herpes Simplex virus glycoprotein D (gD) tag and its antibody (Paborsky et al., Protein Engineering 3:547-553 (1990)). Other tag polypeptides include the Flag-peptide (Hopp et al., BioTechnology 6:1204-1210 (1988)); the KT3 epitope peptide (Martin et al., Science 255:192-194 (1992)); tubulin epitope peptide (Skinner et al., J. Biol. Chem. 266:15163-15166 (1991)); and the T7 gene 10 protein peptide tag (Lutz-Freyermuth et al., Proc. Natl. Acad. Sci. USA 87:6393-6397 (1990)).

In another embodiment, the conjugated compounds may comprise toxins that cause cell death, or impair cell survival when introduced into a cell. A suitable toxin is campylobacter toxin CDT (Lara-Tejero, M., Science 290:354-57 (2000)). Expression of the CdtB subunit, which has homology to nucleases, causes cell cycle arrest and ultimately cell death. Another exemplary toxin is diptheria toxin (and similar Pseudomonas exotoxin), which functions by ADP ribosylating ef-2 (elongation factor 2) molecule in the cell and preventing translation. Entry of the diptheria toxin A subunit induces cell death in cells containing the toxin fragment. Other useful toxins include cholera toxin and pertussis toxin (catalytic subunit-A ADP ribosylates the G protein regulating adenylate cyclase), pierisin from cabbage butterflys, an inducers of apoptosis in mammalian cells (Watanabe, M., Proc. Natl. Acad. Sci. USA 96:10608-13 (1999)), ribosome inactivating toxins (e.g., ricin A chain, Gluck, A. et al., J. Mol. Biol. 226:411-24 (1992)); and nigrin (Munoz, R. et al., Cancer Lett. 167: 163-69 (2001)).

Bioactive compounds suitable for delivery by the compositions herein, include, among others, chemotherapeutic compounds, including by way of example and not limitation, vinblastin, bleomycin, taxol, cis-platin, adriamycin, and mitomycin. Exemplary chemotherapeutic agents suitable for the present purposes are compounds acting on DNA synthesis and stability. For example, anti-neplastic agents of the anthracyclin class of compounds act by causing strand breaks in the DNA and are used as standard therapy against cancer. Exemplary anti-neoplastic agents of this class are daunorubicin and doxorubicin. Coupling of these compounds to proteins, including antibodies, are described in Langer, M. et al., J. Med. Chem. 44(9):1341-1348 (2001) and King, H. D. et al., Bioconjug. Chem. 10:279-288 (1999)). By attaching or linking the antineoplastic agents to the antibodies, the compounds are delivered to progenitor cells with a high degree of specificity and promote killing of the targeted cells.

Other classes of antitumor agents are the enediyne family of antibiotics, representative members of which include calicheamicins, neocarzinostatin, esperamincins, dynemicins, kedarcidin, and maduropeptin (see, e.g., Smith, A. L. and Nicolaou, K. C., J. Med. Chem. 39:2103-2117 (1996)). Similar to doxorubicin and daunorubicin, the antitumor activity of these agents resides in their ability to create strand breaks in the cellular DNA. Conjugates to antibodies have been used to deliver these molecules into those tumor cells expressing antigens recognized by the antibody and shown to have potent antitumor activity with reduced toxicity as compared to the unconjugated compounds (Hinman, L. M. et al., Cancer Res. 53:3336-3342 (1993)). Conjugating the enediyne compounds to the compositions described herein provides another method of targeting committed progenitor cells.

Radioactive compounds are useful as signals (e.g., tracers) or used to provide a therapeutic effect by their delivery to a cell targeted (e.g., in the form of radiopharmaceutical preparations) and may be attached to the antibodies by methods described below. Useful radioactive nuclides include, by way of example and not limitation, ³H, ¹⁴C, ³²P, ³⁵Cr, ⁵⁷Co ⁵⁹Fe, ⁶⁷Ga, ⁸² Rb, ⁸⁹Sr, ⁹⁹Tc, ¹¹¹In, ¹²³I, ¹²⁵I, ¹²⁹I, ¹³¹I, and ¹⁸⁶Re.

The conjugation of compounds to antibodies is well know to the skilled artisan, and typically takes advantage of functional groups present on or introduced onto the antibodies and compound. Functional groups include, among others, hydroxyl, amino, thio, imino, and carboxy moieties. Reaction between functional groups may be aided by coupling reagents and crosslinking agents. Crosslinking agents and linkers and corresponding methods for conjugation are described in Hermanson, G. T., Bioconjugate Techniques, Academic Press, San Diego, Calif. (1996); Aslam M. and Dent A H., Bioconjugation: protein coupling techniques for the biomedical sciences, Houndsmills, England: Macmillan Publishers (1999); Pierce: Applications Handbook & Catalog, Perbio Science, Ermbodegem, Belgium (2003-2004); Haughland, R. P., Handbook of Fluorescent Probes and Research Chemicals Eugene, 9^{th} Ed., Molecular Probes, OR (2003); and U.S. Pat. No. 5,747,641; all references incorporated herein by reference. Exemplary coupling or linking reagents include, by way of example and not limitation, hemi-succinate esters of N-hydroxysuccinimide; sulfo-N-hydroxy-succinimide; hydroxybenzotriazole, and p-nitrophenol; dicyclohexylcarbodiimide (DCC), 1-(3-dimethylaminopropyl)-3-ethylcarbodiimide (ECD), and 1-(3-dimethylaminopropyl)-3-ethylcarbodiimide methiodide (EDCI) (see, e.g., U.S. Pat. No. 4,526,714) the disclosure of which is fully incorporated by reference herein. Other linking reagents include glutathione, 3-(diethoxyphosphoryloxy)-1,2,3-benzotriazin-4(3H)-one (DEPBT), onium salt-based coupling reagents, polyoxyethylene-based heterobifunctional cross-linking reagents, and other reagents that facilitate the coupling of antibodies to organic drugs and peptides and other ligands (Haitao, et al., Organ Lett 1:91-94 (1999); Albericio et al., J Organic Chemistry 63:9678-9683 (1998); Arpicco et al., Bioconjugate Chem. 8:327-337 (1997); Frisch et al., Bioconjugate Chem. 7:180-186 (1996); Deguchi et al., Bioconjugate Chem. 10:32-37 (1998); Beyer et al., J. Med. Chem. 41:2701-2708 (1998); Drouillat et al., J. Pharm. Sci. 87:25-30 (1998); Trimble et al., Bioconjugate Chem. 8:416-423 (1997)).

Techniques for conjugating therapeutic compounds to antibodies are also described in Arnon et al., “Monoclonal Antibodies for Immunotargeting of Drugs in Cancers Therapy,” in Monoclonal Antibodies and Cancer Therapy, Reisfeld et al., ed., pp 243-256, Alan R. Liss, Inc. (1985); Thorpe, et al. “The Preparation and Cytotoxic Properties of Antibody Toxin Conjugates,” Immunol. Rev. 62:119-58 (1982); and Pietersz, G. A., “The linkage of cytotoxic drugs to monoclonal antibodies for the treatment of cancer,” Bioconjugate Chemistry 1(2):89-95 (1990), all references incorporated herein by reference.

### 4.5 Pharmaceutical Compositions

In the preparation of the pharmaceutical compositions comprising the antibodies described in the teachings herein, a variety of vehicles and excipients and routes of administration may be used, as will be apparent to the skilled artisan. Representative formulation technology is taught in, inter alia, Remington: The Science and Practice of Pharmacy, 19th Ed., Mack Publishing Co., Easton, Pa. (1995) and Handbook of Pharmaceutical Excipients, 3rd Ed, Kibbe, A. H. ed., Washington D.C., American Pharmaceutical Association (2000); hereby incorporated by reference in their entirety.

The pharmaceutical compositions will generally comprise a pharmaceutically acceptable carrier and a pharmacologically effective amount of the antibodies, or mixture of antibodies, or suitable salts thereof. Use of the monoclonal antibodies or a mixture of monoclonal antibodies specific to a progenitor cell population as a therapeutic has a number of advantages. Abnormally proliferating cells have a tendency to mutate, and thus may lose the antigen recognized by the monoclonal antibody. Moreover, antigen density in the targeted cell could be low such that there is insufficient triggering of the signals necessary for destruction of the cell by the immune system.

The pharmaceutical composition may be formulated as powders, granules, solutions, suspensions, aerosols, solids, pills, tablets, capsules, gels, topical crémes, suppositories, transdermal patches, and other formulations known in the art.

For the purposes described herein, pharmaceutically acceptable salts of the antibodies is intended to include any art recognized pharmaceutically acceptable salts including organic and inorganic acids and/or bases. Examples of salts include sodium, potassium, lithium, ammonium, calcium, as well as primary, secondary, and tertiary amines, esters of lower hydrocarbons, such as methyl, ethyl, and propyl. Other salts include organic acids, such as acetic acid, propionic acid, pyruvic acid, maleic acid, succinic acid, tartaric acid, citric acid, benzoic acid, cinnamic acid, salicylic acid, etc.

As used herein, “pharmaceutically acceptable carrier” comprises any standard pharmaceutically accepted carriers known to those of ordinary skill in the art in formulating pharmaceutical compositions. Thus, the antibodies, by themselves, such as being present as pharmaceutically acceptable salts, or as conjugates, may be prepared as formulations in pharmaceutically acceptable diluents; for example, saline, phosphate buffer saline (PBS), aqueous ethanol, or solutions of glucose, mannitol, dextran, propylene glycol, oils (e.g., vegetable oils, animal oils, synthetic oils, etc.), microcrystalline cellulose, carboxymethyl cellulose, hydroxyipropyl methyl cellulose, magnesium stearate, calcium phosphate, gelatin, polysorbate 80 or the like, or as solid formulations in appropriate excipients.

The pharmaceutical compositions will often further comprise one or more buffers (e.g., neutral buffered saline or phosphate buffered saline), carbohydrates (e.g., glucose, mannose, sucrose or dextrans), mannitol, proteins, polypeptides or amino acids such as glycine, antioxidants (e.g., ascorbic acid, sodium metabisulfite, butylated hydroxytoluene, butylated hydroxyanisole, etc.), bacteriostats, chelating agents such as EDTA or glutathione, adjuvants (e.g., aluminium hydroxide), solutes that render the formulation isotonic, hypotonic or weakly hypertonic with the blood of a recipient, suspending agents, thickening agents and/or preservatives. Alternatively, compositions of the present invention may be formulated as a lyophilizate.

While any suitable carrier known to those of ordinary skill in the art may be employed in the compositions of this invention, the type of carrier will typically vary depending on the mode of administration. Antibody compositions may be formulated for any appropriate manner of administration, including for example, oral, nasal, mucosal, intravenous, intraperitoneal, intradermal, subcutaneous, and intramuscular administration.

For parenteral administration, the compositions can be administered as injectable dosages of a solution or suspension of the substance in a physiologically acceptable diluent with a pharmaceutical carrier that can be a sterile liquid such as sterile pyrogen free water, oils, saline, glycerol, polyethylene glycol or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, non-aqueous solutions of peanut oil, soybean oil, corn oil, cottonseed oil, ethyl oleate, and isopropyl myristate. Antibodies can be administered in the form of a depot injection or implant preparation which can be formulated in such a manner as to permit a sustained release of the active ingredient. An exemplary composition comprises antibody at 5 mg/ml, formulated in aqueous buffer consisting of 50 mM L-histidine, 150 mM NaCl, adjusted to pH 6.0 with HCl.

Typically, the compositions are prepared as injectables, either as liquid solutions or suspensions; solid or powder forms suitable for reconstitution with suitable vehicles, including by way example and not limitation, sterile pyrogen free water, saline, buffered solutions, dextrose solution, etc., prior to injection. The preparation also can be emulsified or encapsulated in liposomes or micro particles such as polylactide, polyglycolide, or copolymers, as further discussed below (see, e.g., Langer, Science 249:1527 (1990) and Hanes, Advanced Drug Delivery Rev. 28:97-119 (1997)).

Additionally, the compositions may also be introduced or encapsulated into the lumen of liposomes for delivery and for extending their life time ex vivo or in vivo. As known in the art, liposomes can be categorized into various types: multilamellar (MLV), stable plurilamellar (SPLV), small unilamellar (SUV) or large unilamellar (LUV) vesicles. Liposomes can be prepared from various lipid compounds, which may be synthetic or naturally occurring, including phosphatidyl ethers and esters, such as phosphotidylserine, phosphotidylcholine, phosphatidyl ethanolamine, phosphatidylinositol, dimyristoylphosphatidylcholine; steroids such as cholesterol; cerebrosides; sphingomyelin; glycerolipids; and other lipids (see, e.g., U.S. Pat. No. 5,833,948).

Cationic lipids are also suitable for forming liposomes. Generally, the cationic lipids have a net positive charge and have a lipophilic portion, such as a sterol or an acyl or diacyl side chain. Preferably, the head group is positively charged. Typical cationic lipids include 1,2-dioleyloxy-3-(trimethylamino)propane; N-[l-(2,3,-ditetradecycloxy)propyl]-N,N-dimethyl-N-N-hydroxyethylammonium bromide; N-[1-(2,3-dioleyloxy)propyl]-N,N-dimethyl-N-hydroxy ethylammonium bromide; N-[1-(2,3-dioleyloxy) propyl]-N,N,N-trimethylammonium chloride; 3-[N-(N′,N′-dimethylaminoethane)carbamoyl]cholesterol; and dimethyidioctadecylammonium.

Liposomes also include vesicles derivatized with a hydrophilic polymer, as provided in U.S. Pat. No. 5,013,556 and 5,395,619, hereby incorporated by reference, (see also, Kono, K. et al., J. Controlled Release 68: 225-35 (2000); Zalipsky, S. et al., Bioconjug. Chem. 6: 705-708 (1995)) to extend the circulation lifetime in vivo. Hydrophilic polymers for coating or derivation of the liposomes include polyethylene glycol, polyvinylpyrrolidone, polyvinylmethyl ether, polyaspartamide, hydroxymethyl cellulose, hydroxyethyl cellulose, and the like.

Liposomes are prepared by ways well known in the art (see, e.g., Szoka, F. et al., Ann. Rev. Biophys. Bioeng. 9: 467-508 (1980)). One typical method is the lipid film hydration technique in which lipid components are mixed in an organic solvent followed by evaporation of the solvent to generate a lipid film. Hydration of the film in aqueous buffer solution, preferably containing the subject antibodies, results in an emulsion, which is sonicated or extruded to reduce the size and polydispersity. Other methods include reverse-phase evaporation (see, e.g., Pidgeon, C. et al., Biochemistry 26: 17-29 (1987); Duzgunes, N. et al., Biochim. Biophys. Acta. 732: 289-99 (1983)), freezing and thawing of phospholipid mixtures, and ether infusion.

In another embodiment, the carriers are in the form of microparticles, microcapsules, micropheres and nanoparticles, which may be biodegradable or non-biodegradable (see, e.g., “Microencapsulates: Methods and Industrial Applications,” in Drugs and Pharmaceutical Sciences, Benita, S. ed, Vol 73, Marcel Dekker Inc., New York (1996); incorporated herein by reference). As used herein, microparticles, microspheres, microcapsules and nanoparticles mean a particle, which is typically a solid, containing the substance to be delivered. The substance is within the core of the particle or attached to the particle's polymer network. Generally, the difference between microparticles (or microcapsules or microspheres) and nanoparticles is one of size. As used herein, microparticles have a particle size range of about 1 to about >1000 microns. Nanoparticles have a particle size range of about 10 to about 1000 nm.

A variety of materials are useful for making microparticles. Non-biodegradable microcapsules and microparticles include, but not limited to, those made of polysulfones, poly(acrylonitrile-co-vinyl chloride), ethylene-vinyl acetate, hydroxyethylmethacrylate-methyl-methacrylate copolymers. These are useful for implantation purposes where the encapsulated composition diffuses out from the capsules. In another aspect, the microcapsules and microparticles are based on biodegradable polymers, preferably those that display low toxicity and are well tolerated by the immune system. These include protein based microcapsulates and microparticles made from fibrin, casein, serum albumin, collagen, gelatin, lecithin, chitosan, alginate or poly-amino acids such as poly-lysine. Biodegradable synthetic polymers for encapsulating may comprise polymers such as polylactide (PLA), polyglycolide (PGA), poly(lactide-co-glycolide) (PLGA), poly(caprolactone), polydioxanone trimethylene carbonate, polyhybroxyalkonates (e.g., poly(β-hydroxybutyrate)), poly(γ-ethyl glutamate), poly(DTH iminocarbony (bisphenol A iminocarbonate), poly(ortho ester), and polycyanoacrylate. Various methods for making microparticles containing the subject compositions are well known in the art, including solvent removal process (see, e.g., U.S. Pat. No. 4,389,330); emulsification and evaporation (Maysinger, D. et al., Exp. Neuro. 141: 47-56 (1996); Jeffrey, H. et al., Pharm. Res. 10: 362-68 (1993)), spray drying, and extrusion methods.

Another type of carrier is nanoparticles, which are generally suitable for intravenous administrations. Submicron and nanoparticles are generally made from amphiphilic diblock, triblock, or multiblock copolymers as is known in the art. Polymers useful in forming nanoparticles include, but are limited to, poly(lactic acid) (PLA; see Zambaux et al., J. Control Release 60: 179-188 (1999)), poly(lactide-co-glycolide), blends of poly(lactide-co-glycolide) and polycarprolactone, diblock polymer poly(I-leucine-block-I-glutamate), diblock and triblock poly(lactic acid) (PLA) and poly(ethylene oxide) (PEO) (De Jaeghere, F. et al., Pharm. Dev. Technol.; 5: 473-83 (2000)), acrylates, arylamides, polystyrene, and the like. As described for microparticles, nanoparticles may be non-biodegradable or biodegradable. Nanoparticles may be also be made from poly(alkylcyanoacrylate), for example poly(butylcyanoacrylate), in which the therapeutic composition is absorbed onto the nanoparticles and coated with surfactants (e.g., polysorbate 80). Methods for making nanoparticles are similar to those for making microparticles and include, among others, emulsion polymerization in continuous aqueous phase, emulsification-evaporation, solvent displacement, and emulsification-diffusion techniques (see, e.g., Kreuter, J. Nano-particle Preparation and Applications, in Microcapsules and Nanoparticles in Medicine and Pharmacy, pg. 125-148, (M. Donbrow, ed.) CRC Press, Boca Rotan, Fla. (1991); incorporated herein by reference).

The pharmaceutical compositions described herein may be presented in unit-dose or multi-dose containers, such as sealed ampoules or vials. Such containers are typically sealed in such a way to preserve the sterility and stability of the formulation until use. In general, formulations may be stored as suspensions, solutions or emulsions in oily or aqueous vehicles, as indicated above. Alternatively, a pharmaceutical composition may be stored in a freeze-dried condition requiring only the addition of a sterile liquid carrier immediately prior to use.

### 4.6 Use of Antibodies

### 4.6.2 Therapeutic Use of Antibodies

Methods of immunotargeting cancer cells using antibodies or antibody fragments are well known in the art. U.S. Pat. No. 6,306,393 describes the use of anti-CD22 antibodies in the immunotherapy of B-cell malignancies, and U.S. Pat. No. 6,329,503 describes immunotargeting of cells that express serpentine transmembrane antigens. Antibodies described herein (including humanized or human monoclonal antibodies or fragments or other modifications thereof, optionally conjugated to cytotoxic agents) can be introduced into a patient such that the antibody binds to cancer cells and mediates the destruction of the cells and the tumor and/or inhibits the growth of the cells or the tumor. Without intending to limit the disclosure, mechanisms by which such antibodies can exert a therapeutic effect may include complement-mediated cytolysis, antibody-dependent cellular cytotoxicity (ADCC), modulating the physiologic function of the tumor antigen, inhibiting binding or signal transduction pathways, modulating tumor cell differentiation, altering tumor angiogenesis factor profiles, modulating the secretion of immune stimulating or tumor suppressing cytokines and growth factors, modulating cellular adhesion, and/or by inducing apoptosis. The antibodies can also be conjugated to toxic or therapeutic agents, such as radioligands or cytosolic toxins, and may also be used therapeutically to deliver the toxic or therapeutic agent directly to tumor cells.

In addition to the uses above, the compositions have applications to the treatment of conditions or diseases involving cells of the hematopoietic system. The present disclosure further provides methods of using the antibodies to target leukemic stem cells.

The disclosure further provides methods of using the antibodies for treating disorders involving cells of the myeloid and lymphoid lineages. Various diseases have origins in the committed progenitor cell populations, or involve progenitors cell by differentiation of diseased cells through the myeloid or lymphoid pathway.

By “treatment” herein is meant therapeutic or prophylactic treatment, or a suppressive measure for the disease, disorder or undesirable condition. Treatment encompasses administration of the subject antibodies in an appropriate form prior to the onset of disease symptoms and/or after clinical manifestations, or other manifestations, of the disease to reduce disease severity, halt disease progression, or eliminate the disease. Prevention of the disease includes prolonging or delaying the onset of symptoms of the disorder or disease, preferably in a subject with increased susceptibility to the disease.

The antibodies described herein are particularly applicable to the treatment of myeloproliferative disorders, also referred to generally as hematopoietic malignancies, which are proliferative disorders involving cells of the myeloid. The term malignancy refers to growth and proliferation of one or more clones of abnormal cells. Leukemia typically describes a condition in which abnormal cells are present in the bone marrow and peripheral blood.

Myeloproliferative disorders are categorized into three general groups of conditions: dysmyelopoietic disorder, acute myeloproliferative leukemia, and chronic myeloproliferative disorder.

Dysmyelopoietic disease (DMPS) is a condition characterized by presence of megablastoids, megarkaryocyte dysplasia, and an increase in number of abnormal blast cells, reflective of enhanced granulocyte maturation process. Patients with DMPS show chromosomal abonormalities similar to those found in acute myeloid leukemia and progress to acute myeloid leukemia in a certain fraction of afflicted patients (Kardon, N. et al., Cancer 50(12):2834-2838 (1982)).

Acute myeloproliferative leukemia (AML), also known as acute nonlymphocytic leukemia, acute myelocytic leukemia, acute myeloblastic leukemia, and acute granulocytic leukemia, is characterized by the presence of abnormal hematopoietic progenitor cells that have been blocked at an undifferentiated or partially differentiated stage of maturation, and thus are unable to differentiate into myeloid, erythroid, and/or megakaryocytic cell lines. The abnormal cells block differentiation of normal progenitor cells in the bone marrow, resulting in thrombocytopenia, anaemia, and granulocytopenia. Diagnosis of AML is made when at least 30% of nucleated cells in the bone marrow are blasts. Acute myeloid leukemia is further divided into subtypes M1 to M7 based on morphology of the proliferating cells and cytochemical staining properties.

Chronic myeloproliferative disorders are a collection of conditions characterized by increased number of mature and immature granulocytes, erythrocytes, and platelets. Chronic myeloproliferative disorders can transition to other forms within this group, with a tendency to terminate in acute myeloid leukemia. Specific diseases within this group include polycythemia vera, chronic myeloid leukemia, agnogenic myeloid leukemia, essential thrombocythemia, and chronic neutrophilic leukemia.

It is observed that the different categories of myeloproliferative disorders have associated with them genetic abnormalities, and specific chromosomal translocations mark different disease subtypes throughout the progression of the disease. For example, nearly 95% of all patients diagnosed with chronic myeloid leukemia have the translocation between long arms of chromosome 9 and 22 t(9;22) (q34;q11), which results in a fusion between the c-abl gene encoding a non-receptor tyrosine kinase, and the bcr gene encoding a serine-threonine kinase. For acute myeloid leukemia, approximately 60-90% of patients display cytogenetic abnormalities, with about 15% them being translocation t(8;21), which creates an abnormal transcription factor fusion protein between the AML-1 gene and the ETO gene. Another type of translocation seen in AML is t(15; 17), a translocation between chromosomes 15 and 17 that creates a fusion between the promyelocytic leukemia gene and the retinoic acid receptor alpha (RAR-alpha) (Brown, D. et al., Proc. Natl Acad. Sci. USA 94:2551-2556 (1997)).

Various forms of leukemia appear to have their origins in a small population of HSCs or committed myeloid progenitor cells in which the cells acquire a combination of mutations that give rise to the malignant phenotype. The role of HSCs as the origin of some myeloproliferative disorders is suggested from transplantation experiments showing that cells with CD34^{+ CD}38⁻ marker phenotype is able to give rise to AML when transplanted into NOD/SCID immunodeficient mice while transplantation of cells with CD34⁻ CD38⁺ phenotype, characteristic of committed myeloid progenitors, do not give rise to AML (see, e.g., Blair, A. et al., Blood 89:3104-3112 (1997)).

Committed myeloid progenitor cells also appear to have the capability of giving rise to hematologic malignancies. In the AML M3 subtype, or acute promyelocytic leukemia, the cytogenetic abnormality t(15;17) is seen in CD34⁻ CD38⁺ cell populations but not in HSC CD34⁺CD38⁻ populations, indicating that APML arises from cells with a more differentiated phenotype that HSCs. This is further supported by transgenic animal models in which forced expression of the PML/RAR-alpha fusion protein in committed myeloid cells is shown to produce disease characteristic of APML.

Additional evidence for role of committed progenitor cells as the origin of leukemic cells comes from observations in CML in which the translocation t(9;22) is seen in normal HSC population. However, expression levels of the fusion protein BCR-ABL in HSCs are not strongly correlated with disease manifestation (Bedi, A. et al., Blood 81:2898-2902 (1993)). Moreover, transgenic mice constructed to overexpress the BCR-ABL protein in committed myeloid cells leads to the CML phenotype in these animal models (Jaiswal, S. et al., Proc. Natl. Acad. Sci. USA 100:10002-10007 (2003)).

In view of the above, methods of treating myeloproliferative disorders are provided herein using the anti-CD45RA antibody compositions of the present disclosure. The treatments are applicable to myeloid malignancies that involve abnormalities of HSCs and committed progenitor cells. Generally, the methods comprise administration of a therapeutically effective amount of monoclonal antibody, and/or mixtures of monoclonal antibodies directed to specific myeloid progenitor cell populations. If the disorder originates from leukemic HSCs, the antibody treatment should reduce the overall tumor cell burden by eliminating or reducing the number of proliferated cells, thereby allowing normal myeloid cells or lymphocytes to repopulate the hematopoietic organs. The remaining leukemic HSC cells can be treated with agents targeted to the abnormal HSC cell population. Should the leukemic stem cells reside in committed progenitor cells, the antibody treatments should reduce not only the tumor burden but also promote depletion or elimination of leukemic cells having the marker characteristics of committed progenitor cells from which the leukemic cells are derived. As will be apparent to those skilled in the art, unlike other art recognized treatments that target differences between normal and abnormal cells, in some embodiments the antibody compositions of the present disclosure will target both abnormal and normal progenitor cells.

The therapeutic preparations can use nomodified anti-CD45RA antibodies or antibodies conjugated with a therapeutic compound, such as a toxin or cytotoxic molecule, depending on the functionality of the antibody. Generally, when nonmodified antibodies are used, they will typically have a functional Fc region. By “functional Fc region” herein is meant a minimal sequence for effecting the biological function of Fc, such as binding to Fc receptors, particularly FcγR (e.g., FcγRI, FcγRII, and FcγRIII). Without being bound by theory, it is believed that the Fc region may affect the effectiveness of anti-tumor monoclonal antibodies by binding to Fc receptors immune effector cells and modulating cell mediated cytotoxicity, endocytosis, phagocytosis, release of inflammatory cytokines, complement mediate cytotoxicity, and antigen presentation. In this regard, polyclonal antibodies, or mixtures of monoclonals will be advantageous because they will bind to different epitopes and thus have a higher density of Fc on the cell surface as compared to when a single monoclonal antibody is used. Of course, to enhance their effectiveness in depleting targeted cells, or where nonmodified antibodies are not therapeutically effective, antibodies conjugated to toxins or cytotoxic agents may be used. Thus, not only are the antibodies useful as therapeutic molecules themselves, they also find utility in targeted delivery of therapeutic molecules to myeloid cells.

Alternatively, where the antibodies exhibit a direct effect of antigen and/or cell function, enhancement of the Fc receptor functionality may be less significant.

The antibody compositions may be used either alone or in combination with other therapeutic agents to increase efficacy of traditional treatments or to target abnormal cells not targeted by the antibodies. Combining the antibody therapy method with a chemotherapeutic, radiation or surgical regimen may be preferred in patients that have not received chemotherapeutic treatment, whereas treatment with the antibody therapy may be indicated for patients who have received one or more chemotherapies. Additionally, antibody therapy can also enable the use of reduced dosages of concomitant chemotherapy, particularly in patients that do not tolerate the toxicity of the chemotherapeutic agent very well. Furthermore, treatment of cancer patients with the antibody with tumors resistant to chemotherapeutic agents might induce sensitivity and responsiveness to these agents in combination.

In one aspect, the antibodies are used adjunctively with therapeutic cytotoxic agents, including, by way of example and not limitation, busulfan, thioguanine, idarubicin, cytosine arabinoside, 6-mercaptopurine, doxorubicin, daunorubicin, etoposide, and hydroxyurea. Other agents useful as adjuncts to antibody therapy are compounds directed specifically to the abnormal cellular molecule found in the disease state. These agents will be disease specific. For example, for treating chronic myeloid leukemia arising from BCR-ABL activity, one class of useful compounds are inhibitors of abl kinase activity, such as Imatinib, an inhibitor of bcr-abl kinase, and antisense oligonucleotides against bcr (e.g., Oblimersen). Other agents include, among others, interferon-alpha, humanized anti-CD52, deacetylase inhibitor FR901228 (depsipeptide), and the like.

In another aspect, isotopes are attached to the antibodies and/or fragments for therapeutic purposes. By “isotope” is meant atoms with the same number of protons and hence of the same element but with different numbers of neutrons (e.g., ¹H vs.²H or D). The term “isotope” includes “stable isotopes”, e.g. non-radioactive isotopes, as well as “radioactive isotopes”, e.g. those that decay over time, and radioactive radionuclides. In one embodiment, the antibodies and/or fragments are labeled with a radioisotope, which are useful in radioimmunotherapy. Suitable radioisotopes include without limitation an alpha-emitter, a beta-emitter, and an Auger electron-emitter (Behrt, T. et al., (2000). Eur. J. Nuclear Med. vol. 27 (7):753-765; Vallabhajosula, S. et al., J. Nucl. Med. April (2005); 46(4):634-41). Such radioisotopes include without limitation [65]Zinc, [140]neodymium, [177]lutetium, [179]lutetium, [176m]lutetium, [67]gallium, [159]gallium, [161]terbium, [153]samarium, [169]erbium, [175]ytterbium, [161]holmium, [166]holmium, [167]thulium, [142]praseodymium, [143]praseodymium, [145]praseodymium, [149]promethium, [150]europium, [165]dysprosium, [111]indium, [131]iodine, [125]iodine, [123]iodine, [88]yttrium and [90]yttrium. Suitable radioactive radionuclides include without limitation, ⁶⁷Cu, ⁹⁰Y, ¹²⁵I, ¹³¹I, ¹⁸⁶Re, ¹⁸⁸Re, ²¹¹At, ²¹²Bi. These and other uses of the antibodies will be apparent to those of ordinary skill in the art.

### 4.7 Administration and Dosages

The amount of the compositions needed for achieving a therapeutic effect will be determined empirically in accordance with conventional procedures for the particular purpose. Generally, for administering the compositions ex vivo or in vivo for therapeutic purposes, the compositions are given at a pharmacologically effective dose. By “pharmacologically effective amount” or “pharmacologically effective dose” is an amount sufficient to produce the desired physiological effect or amount capable of achieving the desired result, particularly for treating or retreating the disorder or disease condition, including reducing or eliminating one or more symptoms or manifestations of the disorder or disease. As an illustration, administration of antibodies to a patient suffering from a myeloproliferative disorder provides a therapeutic benefit not only when the underlying disease is eradicated or ameliorated, but also when the patient reports a decrease in the severity or duration of the symptoms associated with the disease. Therapeutic benefit also includes halting or slowing the progression of the underlying disease or disorder, regardless of whether improvement is realized.

The amount administered to the host will vary depending upon what is being administered, the purpose of the administration, such as prophylaxis or therapy, the state of the host, the manner of administration, the number of administrations, interval between administrations, and the like. These can be determined empirically by those skilled in the art and may be adjusted for the extent of the therapeutic response. Factors to consider in determining an appropriate dose include, but is not limited to, size and weight of the subject, the age and sex of the subject, the severity of the symptom, the stage of the disease, method of delivery of the agent, half-life of the agents, and efficacy of the agents. Stage of the disease to consider includes whether the disease is acute or chronic, relapsing or remitting phase, and the progressiveness of the disease. Determining the dosages and times of administration for a therapeutically effective amount are well within the skill of the ordinary person in the art.

For any compositions of the present disclosure, the therapeutically effective dose is readily determined by methods well known in the art. For example, an initial effective dose can be estimated from cell culture or other in vitro assays. For example, Sliwkowsky, M. X. et al., Semin. Oncol. 26(suppl. 12) 60-70 (1999) describes in vitro measurements of antibody dependent cellular cytoxicity. A dose can then be formulated in animal models to generate a circulating concentration or tissue concentration, including that of the IC₅₀ as determined by the cell culture assays.

In addition, the toxicity and therapeutic efficacy are generally determined by cell culture assays and/or experimental animals, typically by determining a LD₅₀ (lethal dose to 50% of the test population) and ED₅₀ (therapeutically effectiveness in 50% of the test population). The dose ratio of toxicity and therapeutic effectiveness is the therapeutic index. Preferred are compositions, individually or in combination, exhibiting high therapeutic indices. Determination of the effective amount is well within the skill of those in the art, particularly given the detailed disclosure provided herein. Guidance is also found in standard reference works, for example Fingl and Woodbury, General Principles In: The Pharmaceutical Basis of Therapeutics pp. 1-46 (1975), and the references cited therein.

To achieve an initial tolerizing dose, consideration is given to the possibility that the antibodies may be immunogenic in humans and in non-human primates. The immune response may be biologically significant and may impair the therapeutic efficacy of the antibody even if the antibody is partly or chiefly comprised of human immunoglobulin sequences such as, for example, in the case of a chimeric or humanized antibody. Within certain embodiments, an initial high dose of antibody is administered such that a degree of immunological tolerance to the therapeutic antibody is established. The tolerizing dose is sufficient to prevent or reduce the induction of an antibody response to repeat administration of the committed progenitor cell specific antibody.

Preferred ranges for the tolerizing dose are between 10 mg/kg body weight to 50 mg/kg body weight, inclusive. More preferred ranges for the tolerizing dose are between 20 and 40 mg/kg, inclusive. Still more preferred ranges for the tolerizing dose are between 20 and 25 mg/kg, inclusive.

Within these therapeutic regimens, the therapeutically effective dose of antibodies is preferably administered in the range of 0.1 to 10 mg/kg body weight, inclusive. More preferred second therapeutically effective doses are in the range of 0.2 to 5 mg/kg body weight, inclusive. Still more preferred therapeutically effective doses are in the range of 0.5 to 2 mg/kg, inclusive. Within alternative embodiments, the subsequent therapeutic dose or doses may be in the same or different formulation as the tolerizing dose and/or may be administered by the same or different route as the tolerizing dose.

For the purposes of this invention, the methods of administration are chosen depending on the condition being treated, the form of the subject antibodies, and the pharmaceutical composition. Administration of the antibody compositions can be done in a variety of ways, including, but not limited to, continuously, subcutaneously, intravenously, orally, topically, transdermal, intraperitoneal, intramuscularly, and intravesically. For example, microparticle, microsphere, and microencapsulate formulations are useful for oral, intramuscular, or subcutaneous administrations. Liposomes and nanoparticles are additionally suitable for intravenous administrations. Administration of the pharmaceutical compositions may be through a single route or concurrently by several routes. For instance, intraperitoneal administration can be accompanied by intravenous injections. Preferably the therapeutic doses are administered intravenously, intraperitonealy, intramuscularly, or subcutaneously.

The compositions may be administered once or several times. In some embodiments, the compositions may be administered once per day, a few or several times per day, or even multiple times per day, depending upon, among other things, the indication being treated and the judgement of the prescribing physician.

Administration of the compositions may also be achieved through sustained release or long-term delivery methods, which are well known to those skilled in the art. By “sustained release or” “long term release” as used herein is meant that the delivery system administers a pharmaceutically therapeutic amount of subject compounds for more than a day, preferably more than a week, and most preferable at least about 30 days to 60 days, or longer. Long term release systems may comprise implantable solids or gels containing the antibodies, such as biodegradable polymers described above (Brown, D. M. et al., Anticancer Drugs 7: 507-513 (1996)); pumps, including peristaltic pumps and fluorocarbon propellant pumps; osmotic and mini-osmotic pumps; and the like.

The method of the invention contemplates the administration of anti-CD45Ra antibodies and in particular any antibody that recognizes the particular antigens recognized by 17.1, as well as combinations of different mAbs. Two or more monoclonal antibodies may provide an improved effect compared to a single antibody. Alternatively, a combination of an antibody with an antibody that binds a different antigen may provide an improved effect compared to a single antibody. Such mAb cocktails may have certain advantages inasmuch as they contain mAbs, which exploit different effector mechanisms or combine directly cytotoxic mAbs with mAbs that rely on immune effector functionality. Such mAbs in combination may exhibit synergistic therapeutic effects.

### 4.8 Kits

The present invention further provides methods to identify the presence of an antigen using the compositions of the present invention, optionally conjugated or otherwise associated with a suitable label. Such methods comprise incubating a test sample with one or more of the anti-CD45RA antibodies of the present invention and assaying for binding antibodies to components within the test sample. Conditions for incubating the antibody with a test sample may vary. Incubation conditions depend on the format employed in the assay, the detection methods employed, and the antibody used in the assay. One skilled in the art will recognize that any one of the commonly available immunological assay formats can readily be adapted to employ antibodies of the present invention (see Chard, T., An Introduction to Radioimmunoassay and Related Techniques, Elsevier Science Publishers, Amsterdam, The Netherlands (1986); Bullock, G. R. et al., Techniques in Immunocytochemistry, Academic Press, Orlando, Fla. Vol. 1 (1982), Vol. 2 (1983), Vol. 3 (1985); Tijssen, P., Practice and Theory of immunoassays: Laboratory Techniques in Biochemistry and Molecular Biology, Elsevier Science Publishers, Amsterdam, The Netherlands (1985). The test samples of the present invention include cells, protein or membrane extracts of cells, or biological fluids such as sputum, blood, serum, plasma, lymphatic fluid, or urine. The test sample used in the above-described method will vary based on the assay format, nature of the detection method and the tissues, cells or extracts used as the sample to be assayed. Methods for preparing protein extracts or membrane extracts of cells are well known in the art and can be readily be adapted in order to obtain a sample which is compatible with the system utilized.

In another embodiment of the present invention, kits are provided which contain the necessary reagents to carry out the assays of the present invention. Specifically, the invention provides a compartment kit to receive in one or more containers which comprises: (a) a first container comprising one of the anti-CD45RA antibodies or complexes of the present invention; and (b) one or more other containers comprising one or more of the following: wash reagents, reagents capable of detecting presence of the antibody. A compartment kit includes any kit in which reagents are contained in separate containers. Such containers include small glass containers, plastic containers or strips of plastic or paper. Such containers allows one to efficiently transfer reagents from one compartment to another compartment such that the samples and reagents are not cross-contaminated, and the agents or solutions of each container can be added in a quantitative fashion from one compartment to another. One skilled in the art will readily recognize that the disclosed antibodies of the present invention can be readily incorporated into one of the established kit formats which are well known in the art.

Provided herein are kits which include a composition described herein. In some embodiments the kit comprising a hybridoma, antibody and/or mixtures of antibodies disclosed herein. The kit would preferably provide a pharmaceutical formulation. In some embodiments, the kits contain at least one additional reagent, including other antibodies, including other monoclonal antibodies directed to HSCs, committed progenitor cells, polyclonal antibodies, or mixtures of the antibodies as reagents for detection myeloid cell types. Frozen or fixed forms of HSCs, CMPs, GMP and/or MEPs reactive with the antibodies and reagents form additional contents of the kits. The kit typically contains containers which may be formed from a variety of materials such as glass or plastic, and can include for example, bottles, vials, syringes, and test tubes. A label typically accompanies the kit, and includes any writing or recorded material, which may be electronic or computer readable form (e.g., disk, optical disc, or tape) providing instructions or other information for used of the contents of the kit. The label indicates that the formulation is used for diagnosing or treating the disorder of choice.

### 5. EXAMPLES

### 5.1 Example 1

### Production and Characterization of Monoclonal Antibodies

The hybridoma for producing the monoclonal antibody designated 17.1 was deposited with American Type Culture Collection under Accession Number PTA-7339.

The class of antibodies produced by the 17.1 hybridoma was determined as follows. Flat bottom, 96-well plates were coated with 100 μls of supernatant from the hybridoma designated as 17.1. Monoclonal antibody 17.1 was detected with a goat anti-mouse IgG, (H+L), peroxidase conjugated in an ELISA assays. This assay indicates that the monoclonal antibody 17.1 is of the class of IgG antibodies.

The isotype of the antibodies produced by the hybridomas was determined as follows. Isotype was determined using an immunochromatographic strip (ICS) tests also called Lateral Flow test). The antibody was solubilized with antibody-coated latex beads, and the complex was allowed to migrate through the strip by capillary action. The complex stopped when it binds to the particular isotype of the sample (IgG1, IgG2a, IgG2b, IgG3, IgA, IgM). Monoclonal antibody 17.1 is mouse IgG2b, kappa. Monoclonal antibody 17.1 is mouse IgG₂b isotype. Generally, mouse IgG₂b isotypes are known in the art to not mediate Fc effector functions because they do not have high affinity to Fc receptors on effector cells.

### 5.2 Example 2

### Monoclonal Antibody Specificity to Human Stem Cells and Progenitors Cells

Antibody binding to HSC, GMP and CMP cells was assessed by multicolor FACS analysis. HSCs and committed myeloid progenitor cell populations were isolated using antibodies to cell surface markers and FACS sorting following conventional techniques. The source of cells is from human mobilized peripheral blood.

Specificity of antibody 17.1 to HSC, GMP and CMP cells was assessed by multicolor FACS analysis using PE-A conjugated Goat anti-mouse IgG monoclonal antibody (PE-A G anti-M) and FITC-A CD45RA staining.

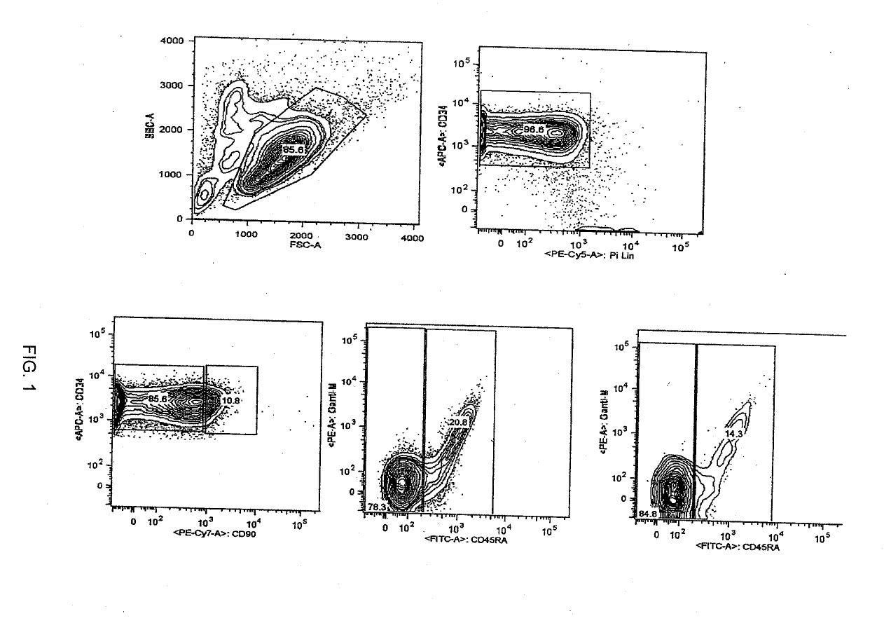

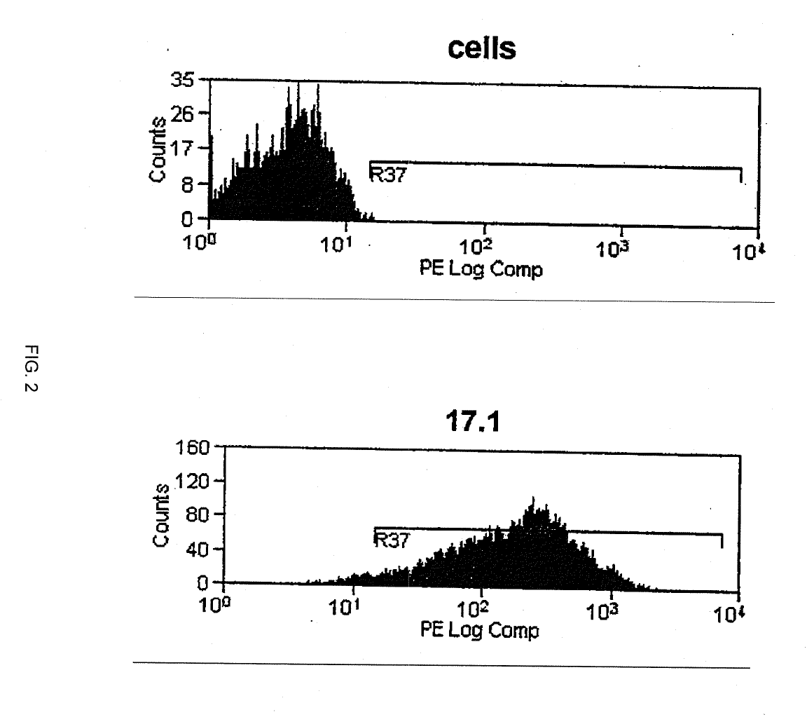

FIG. 1A-E show FACS analysis of the antibody produced by the hybridoma cell line 17.1. FIG. 1C, shows the percentage of HSC and MP in the CD34 enriched sample, the number in the right box indicate the percentage of HSC (CD90⁺ CD34⁺) and number in the left box indicate the percentage of MP (CD34⁺CD90⁻) populations in the sample. FIG.1D, shows the binding of 17.1 monoclonal antibody to CMP/MEP (CD34⁺CD90⁻CD45RA) in the left box and binding of 17.1 to GMP (CD34⁺CD90⁻CD45RA⁺) populations in the right box. FIG. 1E, shows the binding of 17.1 monoclonal antibody to HSC (CD34⁺CD90⁺CD45RA⁻) in the left box and binding of 17.1 to GMP (CD34⁺ CD45RA⁺) populations in the right box.

FIG. 1A-E shows FACS analysis of the antibody produced by the hybridoma cell line 17.1. In FIG. 1A-E, the Y axis shows PE-A G anti-M staining with antibodies produced by the hybridomas cell lines and the X axis shows FITC-A CD45RA staining. The mean fluorescent intensity and percent populations stained with the hybridomas were determined from the contour plots.

The data from FIG. 1A-E is summarized in Table 1, wherein “−“represents no immunoreactivity or non-specific binding, “+” represents minimal immunoreactivity having about 10-25% immunoreactivity, and “++”,“+++”, and “++++” represents specific immunoreactivity, about 25-45%; about 45-65%; and 65-100% immunoreactivity, respectively.

TABLE 1

| | HSC | GMP | GMP | CMP |
|---|---|---|---|---|
| | (CD34⁺CD90⁺ | (CD34⁺CD90⁺ | (CD34⁺CD90⁻ | (CD34⁺CD90⁻ |
| mAb | CD45RA⁻) | CD45RA⁺) | CD45RA⁺) | CD45RA⁻) |
| 17.1 | − | ++++ | ++++ | − |

### 5.3 Example 3

### Monoclonal Antibody Specificity to Cell Lines

Antibody specificity to transformed cell populations was assessed by PE-conjugated Goat anti-mouse secondary to detect the binding or FITC conjugated anti-mouse secondary antibodies and FACS analysis as described above. The cell lines KG-1a, K562, Pfeiffer, RL, and Jurkat cell were purchased from ATCC.

Antibody 17.1 immunoreactivity to KG1-a cells (data not shown). KG-1a cells are human acute myelogenous leukemia cell line. K562 is a chronic myelogenous leukemia (CML) cell line. KG-1a cells are typically are CD34⁺, CD45RA⁺, CD123⁺, CD33⁺, CD13⁺, and CD15⁺.

Antibody 17.1 immunoreactivity to K-562 cells (data not shown). K562 cells are typically CD34⁻, CD13⁺, CD45⁺, CD42⁺, CD71⁺, and GlyA⁺.

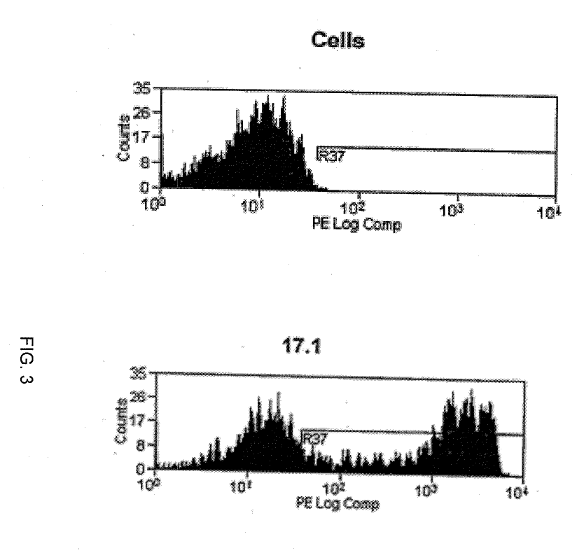

FIG. 2 shows antibody 17.1 immunoreactivity to Jurkat cells. Jurkat is a human T cell leukemia cell line. Jurkat cells are typically CD3⁺, CD45⁺, CD45RA⁺, CD38⁺, HLA-Class I⁺, HLA-Class II⁻, CD123⁻, CD127⁻, CD13⁻, CD33⁻, and CD34⁻.

The 17.1 antibody demonstrates immunoreactivity to the Pfeiffer cell line by way of a FACS analysis as described above (data not shown). The Pfeiffer cell line is a model system for studying non-Hodgkin lymphomas. Pfeiffer cells are typically CD10⁺, CD19⁺, CD20⁺, CD38⁺, CD23⁻, and CD39⁻. According to the results of the FACS, the 17.1 antibody that specifically binds to CD45RA antigen is immunoreactive with Pfeiffer cells at various concentrations (e.g. 0.1, 0.1, 0.5,1, and 10 μg/ml). The control IgG2b is not immunoreactive with Pfeiffer cells.

Immunoreactivity of the 17.1 antibody to the RL cell line. The RL cell line is a B lymphoblast cell line derived from the ascites of a patient with non-Hodgkin lymphoma (NHL). RL cells are typically CD19⁺, CD20⁺, CD21⁺, CD22⁺, HIe-1⁻, HLA DQ⁻, HLA DR⁺, CD25⁻, and T cell receptor (TCR)⁻. According to the results of the FACS (data not shown), the 17.1 antibody that specifically binds to CD45RA antigen is not immunoreactive with RL cells at concentrations of 0.1, 0.1, 0.5, 1, and 10 μg/ml. The control IgG2b is also not immunoreactive with RL cells.

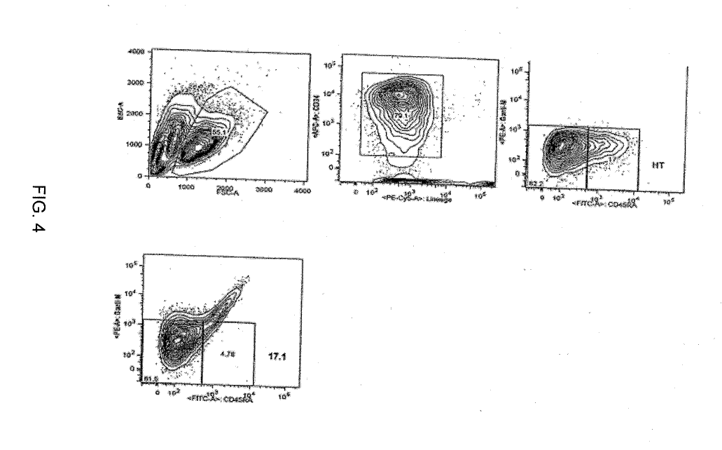

FIG. 3 shows antibody 17.1 immunoreactivity to PMBC cells. PBMC are peripheral blood mononuclear cells prepared from normal donor peripheral blood by density based cell separation using Ficol-hypaque.

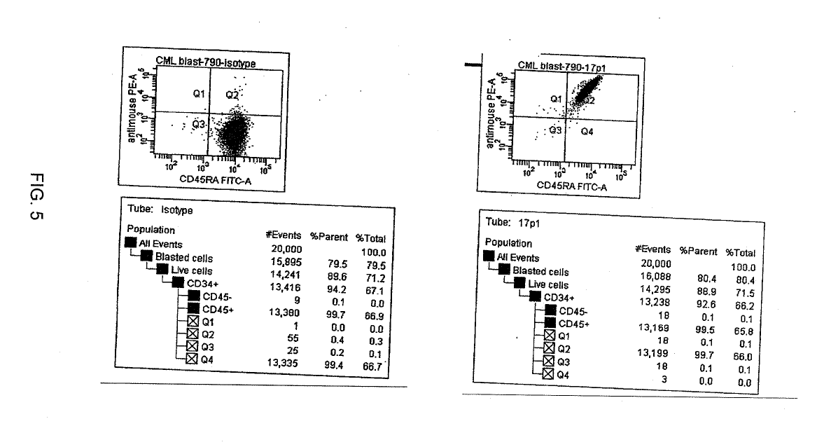

FIG. 4 show antibody 17,1 binding to CML blasts. CML Blast cells are chronic myeloid leukemia (CML) blast cells. CML blasts cells are typically CD34⁺, CD45RA⁺, CD123⁺, CD13⁺, CD15⁺, CD33⁺, and CD11c⁺.

The data described in this Example 3 is summarized in Table 2. Additional studies as described above were performed on Kasumi-3 , Kasumi 4, MEG-01, KU812, and HL-60 cells. In Table 2, “±” represents inconclusive immunoreactivity, “−” represents no immunoreactivity or non-specific binding, “+” represents minimal immunoreactivity having about 10-25% immunoreactivity, and “++”, “+++”, and “++++” represents specific immunoreactivity, having about 25-45%; 45-65%; and 65-100% immunoreactivity, respectively.

TABLE 2

| | KG- | K- | | | CML | | | MEG- | | HL- |
|---|---|---|---|---|---|---|---|---|---|---|
| mAb | 1a | 562 | Jurkat | PBMC | Blast | Kaumi-3 | Kasumi-4 | 01 | KU812 | 60 |
| 17.1 | +++ | + | + | + | +++ | + | ++ | + | + | − |

Table 3 shows the binding characteristics of antibody 17.1 to normal PMBC cells (percent binding). Cells were added at 2×10⁵-5×10⁵ cells/well to a 96-well plate and centrifuge at 1200 RPM for 5 min. Cells were resuspended in 100 μl of a 1:50 dilution of Rat IgG for 20 min. at 4° C. This step helps eliminate non-specific binding of 1° and 2° antibodies by binding to the Fc receptors. Cells were washed with 150 μl of Staining media, centrifuged and resuspend in 100 μl of monoclonal antibody 17.1, 15.1, 181.2, or 178.5.1. The cells were incubated at 4° C. for 20 minutes, washed with 150 μl of Staining media. Added 100 μl of a 1:100 PE-conjugated goat anti-mouse secondary antibody and incubated on ice for 20 minutes in the dark. Washed 2 two times with 150 μl of staining media. The extra wash step was included to remove residual anti-mouse PE. Added 100 μl (1:100 dilution) of FITC or APC conjugated Lineage antibodies (CD4, CD8, CD19, CD15, CD33 from Caltag Laboratories and CD11b, CD56, CD14 from BD Pharmingen) to appropriate samples and incubated for 20 min. at 4° C. in the dark. Cells were washed and resuspended in 100 μl staining media, transfer to FACS tubes, and added 100 μl of 1:500 dilution of Propidium Iodide to eliminate dead cells from the stained samples. Lymphoid and myeloid lineage positive cell populations were gated separately based on the forward and side scatter characteristics using Aria FACS and Diva software. Percentages of lymphoid and myeloid lineage positive cells stained with the monoclonal antibodies were assessed from the gated populations.

TABLE 3

| mAb | CD4⁺ | CD8⁺ | CD11b⁺ | CD14⁺ | CD15⁺ | CD19⁺ | CD33⁺ | CD56⁺ |
|---|---|---|---|---|---|---|---|---|
| 17.1 | 42.3% | 90.2% | 14.4% | 16.4% | 4.0% | 98.9% | 8.7% | 98.4% |

### 5.4 Example 4

### Monoclonal Antibody Specificity to CML Primary Cells

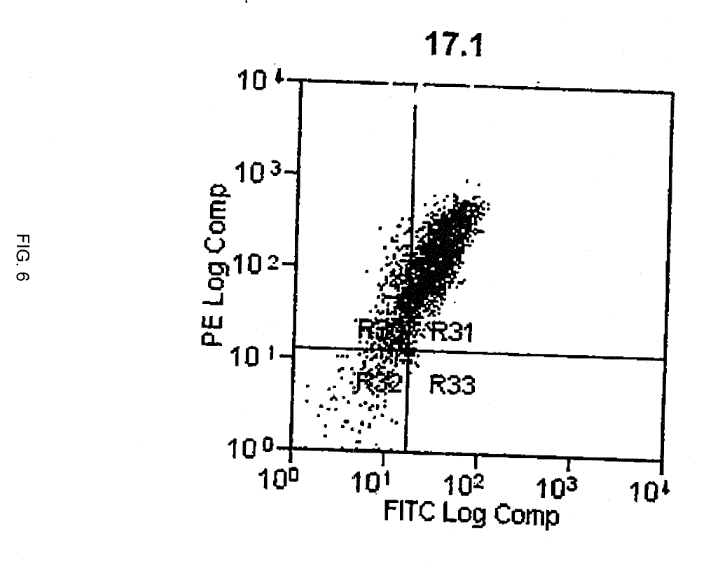

The immunoreactivity of monoclonal antibody 17.1 to primary cells from CML blast patients was determined. FIG. 5 shows the immunoreactivity of monoclonal antibody 17.1 to CD34⁺ primary cells from a single CML patient #790. Table 4 summarizes the specificity of monoclonal antibody 17.1 to primary cells from five CML patients as percent binding.

TABLE 4

| Patient | |
|---|---|
| No. | 17.1 Antibody |
| 649 | 15% |
| 268 | 71% |
| 522 | 44% |
| 222 | 93% |
| 790 | 99% |

### 5.5 Example 5

### Monoclonal Antibody Specificity to AML Primary Cells

The immunoreactivity of monoclonal antibody 17.1 to primary cells from AML blast patients was determined. FIG. 6 shows the specificity of monoclonal antibody 17.1 to CD34⁺ primary cells from a single AML patient #33. Table 5 summarizes the specificity of monoclonal antibody 17.1 to primary cells from two AML patients, as percent binding.

TABLE 5

| Patient | |
|---|---|
| No. | 17.1 Antibody |
| 33 | 80% |
| 263 | 48% |

### 5.6 Example 6

### In Vitro Determination of Cytolytic Activity of Monoclonal Antibodies

Cytolytic activity of monoclonal antibodies is tested in vitro through EU fluorescence release assays, DELFIA EuTDA Cytotoxicity assays (PerkinElmer, Inc.) using human leukemic cell lines or primary cells from CML and/or AML patients. Activities that can be measured in vitro include cell dependent (Antibody Dependent Cell Cytotoxicity, ADCC) and cell independent (Complement Dependent Cytotoxicity, CDC) cytotoxicity as well as direct inhibition of tumor cell proliferation and/or apoptosis.

A number of standard cellular immunology protocols for assaying ADCC and CDC are available and are well known in the art. See Kawamoto, T. et al, In Vitro Cell . Dev. 28A: 782-786 (1992); Cragg, M. et al, Blood 103: 2738-2743 (2004); Palath, V. et al, The Journal of Immunology 148: 3319-3326 (1992); Viasveld L T. et al, Cancer Immunol Immunother. 40: 37-47 (1995) For example, labeled target cells can be cultured with a mAb and control non-specific, isotype matched antibody in the presence of effector cells (NK cells, T cells, monocytes, and/or neutrophils) to determine target cell lysis by ADCC. CDC can be measured by the addition of complement or untreated serum instead of effector cells.

ADCC and CDC: Materials and Methods. Tumor targets were labeled with Europium, DElfia Eu TDA Cytotoxicity reagents (PerkinElmer Inc.) by incubating 1×10⁶ cells/ml of culture medium with 5 μl of fluorescence enhancing ligand (DELFIA BATDA). Cells were incubated at 37° C. for 30 min. Labeled cells were washed 3 times with PBS, re-suspended in RPMI culture medium and plated in 96 V-bottom plates at a concentration of 1×10⁴ cells/100 μls/well in triplicates. mAb17.1 and Isotype control were added to the target cells in different concentration. Effector cells were added to the target cells at specified ratios (10:1 or 40:1) and incubated at 37° C. for 4 hours, 5% CO₂. At the end of the assay, supernatants were collected and measured the fluorescence released using time-resolved fluorometer: Victor 3 (PerkinElmer Inc.) Percentage of cellular cytotoxicity was calculated as % specific lysis=(experimental release−spontaneous release)/(maximal release−spontaneous release) times 100. Maximal fluorescence release is determined by adding lysis buffer to target cells and basal release is measured in the absence of sensitizing antibodies and effector cells.

Isolation of mononuclear and neutrophil effector cells. Ten to 20 mL of peripheral blood is drawn from healthy adult volunteers and mononuclear cells (MNCs) are isolated from citrate or EDTA-anticoagulated blood layered over a discontinuous (70-62%) Percoll (Biochrom, Berlin, Germany) gradient layered over bovine calf serum. After centrifugation, MNCs are collected from the serum/Percoll interface. The remaining erythrocytes are removed by hypotonic lysis with ammonium chloride solution. Purity of MNCs are determined by cytospin preparations or by FACS analysis and should exceeded 95%. MNCs typically contain approximately 60% CD3+ T cells, 20% CD56+ natural killer (NK) cells, and approximately 10% CD14-expressing monocytes, as determined by immunofluorescence. Viability of cells is tested by trypan blue exclusion and should be higher than 95% before use in the assay.

Antibody Dependent Cell Cytotoxicity Assay. Briefly, target cells are labeled with 5 μls of fluorescence enhancing ligand DELFIA BATDA for 30 minutes. After extensive washing with cell culture media, labeled cells are adjusted to 10⁵/mL. Isolated effector cells (100 μL), monoclonal antibody at a concentration of 1 to 20 μg/mL, and culture media were added to 96 well round-bottomed microtiter plates (Nunc). Assays were started by adding target cells (100 μL), resulting in a final volume of 200 μL and an effector-to-target (E/T) cell ratio of 40:1 to 10:1 with isolated effector cells. After 4 hours incubation in a humidified chamber at 37° C., assays are stopped by centrifugation, and fluorescence release from triplicates are measured. Percentage of cellular cytotoxicity was calculated with the following formula: % specific lysis=(experimental release−Spontaneous release)/(maximal release−Spontaneous release)×100. Maximal fluorescence release is determined by adding lysis buffer to target cells and basal release is measured in the absence of sensitizing antibodies and effector cells

Complement Dependent Cytotoxicity Assay. Target cells and monoclonal antibodies were prepared as described above. The monoclonal antibody at a concentration of 1 to 20 μg/mL and pre-tested complements (100 μls) were added to 96 well round-bottomed microtiter plates (Nunc). Assays were started by adding target cells (100 μL), resulting in a final volume of 200 μL. Cultures were incubated for 30-60 minutes at 37° C. in a humidified chamber. Assays were stopped by centrifugation, and fluorescence release from triplicates were measured. Percentage of cytotoxicity was calculated as: % specific lysis=(experimental release−spontaneous release)/(maximal release−spontaneous release)×100. Maximal fluorescence release is determined by adding lysis buffer to target cells and basal release is measured in the absence of sensitizing antibodies and complement.

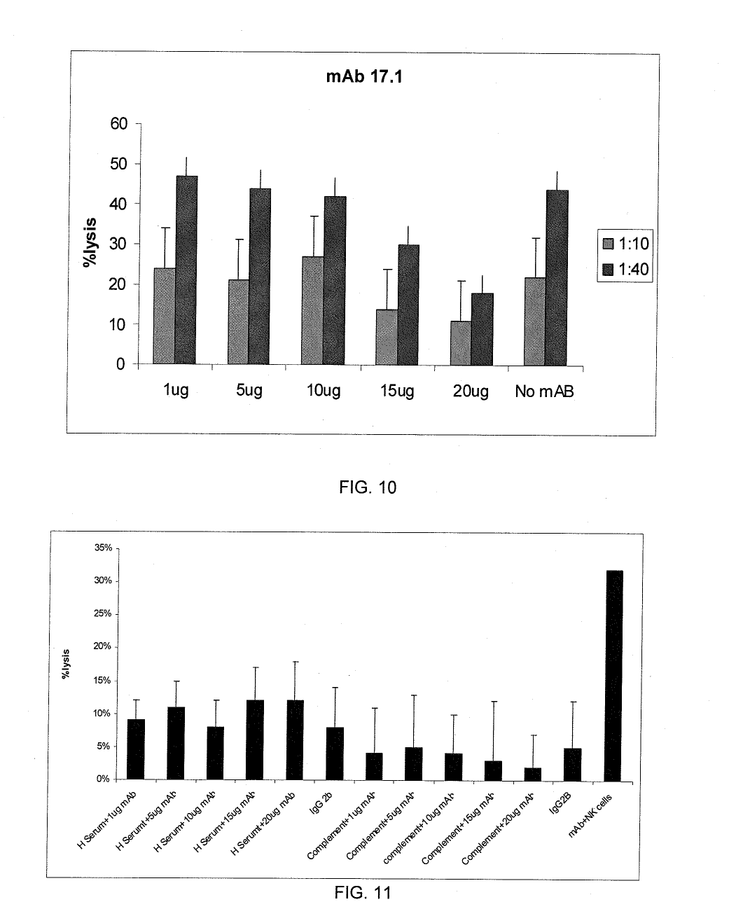

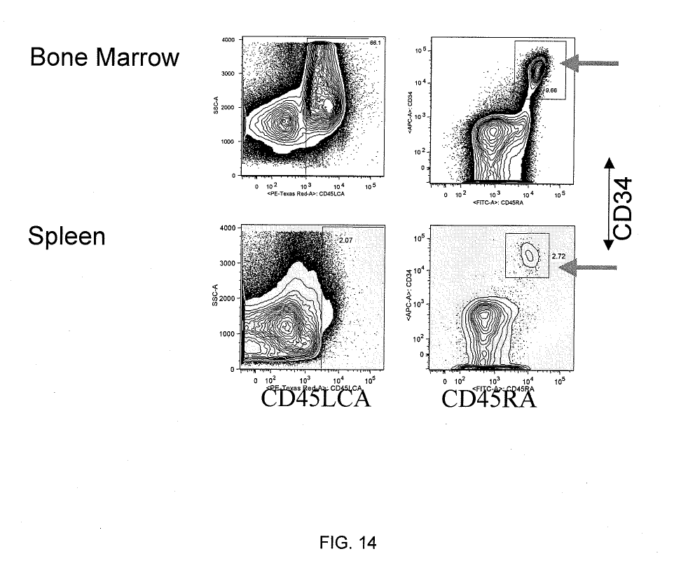

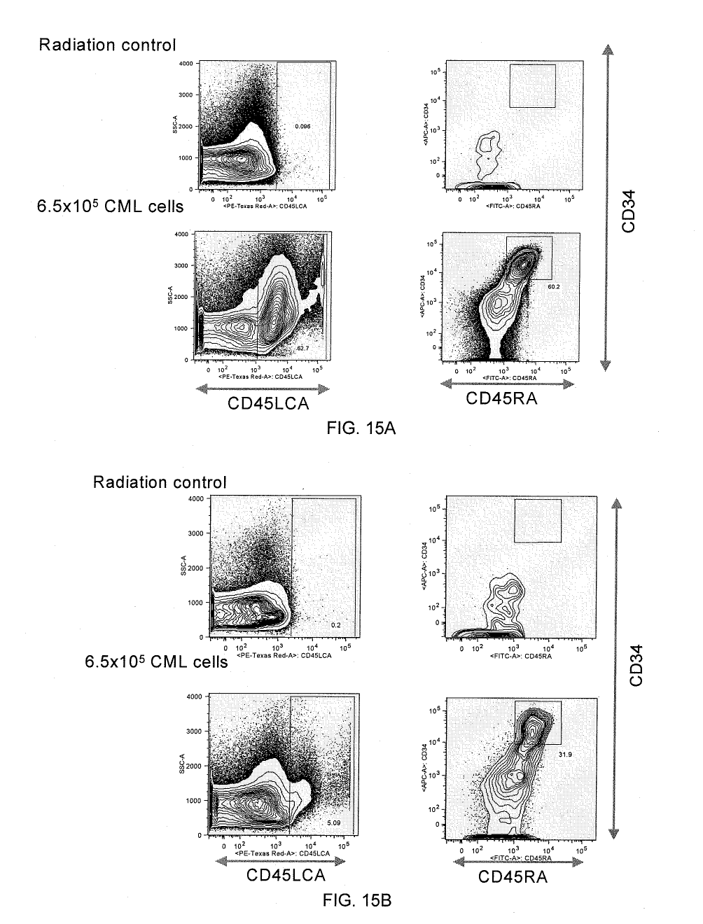

FIG. 10 show no induction of ADCC with mAb 17.1 in Kasumi-4 cells. FIG. 14 shows the percent lysis with 1, 5, 10, 15, 20 μg mAb 17.1 as compared to NK cells alone (no mAb). In the ADCC assay there was no addition lysis by the addition of mAb 17.1 as compared to NK cells alone.

FIG. 11 show no induction of CDC with mAb 17.1 in Kasumi-4 cells. FIG. 15 shows the percent lysis with 1, 5, 10, 15, 20 μg mAb 17.1 with serum or complement. In the CDC assay there was no lysis compared to IgG₂b isotype control (Caltag Laboratories, Burlingame, Calif., Catalog #:MG2b00).

Monoclonal antibody 17.1 is mouse IgG₂b isotype. Generally, mouse IgG₂b isotypes are known in the art to not mediate Fc effector functions because they do not have high affinity to Fc receptors on effector cells.

In vitro tumor cell Proliferation and Inhibition assay: In vitro tumor cell proliferation and inhibition was assessed by using CFSE Cell Proliferation Kit from Molecular Probes (Cat #C34554). CFSE passively diffuses in to cells. It is colorless and non-fluorescent until the acetate groups are cleaved by intracellular esterases to yield highly fluorescent carboxyfluorescein succinimidyl ester. The succinimidyl ester group reacts with intracellular amines, forming fluorescent conjugates that are retained and excess unconjugated reagents and by-products passively diffuse to the extracellular medium, where they can be washed away.

In order to assess the inhibition of proliferation of tumor cells with mAb 17.1, 2×10⁶ Kasumi-4 cells were incubated in 200 μls of PBS with 1-20 μg/mL. of mAb 17.1 in 96-well flat bottom culture plates. Mouse IgG₂b isotype at different concentrations (1-20 μg/mL) was added to different cell culture wells as negative control. The mixture was incubated for 30 minutes on ice and washed 2 times in wash buffer. Cells were resuspended in 1 mL. of staining buffer and added 2 μL. (5 mM stock solution) of CFSE solution to each wells of 6-well plates. Plates were incubated for 10 minutes at 37° C. and quenched the staining by the addition of 5 volumes of ice-cold PBS to the cells. Incubated another 5 minutes on ice and pelleted the cells, washed×3 in PBS. Stained cells were cultured in 1%, 5% and 10% FBS, RPMI 1640 medium for 72 hours at 37° C., 5% CO₂ incubator. After 72 hours of incubation, cells were washed twice with cold wash buffer and resuspended in 1 mL. of 1×PBS . Transfered 100 μls (2×10⁵ cells) of the cell suspension to 5 mL culture tube and analyzed the samples by flow cytometry to assess proliferation.

Inhibition of the tumor cell line, Kasumi-4 cells, proliferation with 17.1 mAb. Monoclonal antibody 17.1 caused a dose dependent increase in the percent inhibition of Kasumi-4 cell proliferation as compared to no antibody and IgG₂b isotype control (data not shown).

Cytolytic activity of monoclonal antibodies was also tested in vitro through an apoptosis assay using Kasumi-4 cells, primary cells from CML patients, and PBMCs. A number of standard cellular protocols for assaying apoptosis are well known in the art. As shown below, 17.1 mAb induced apoptotic cell death in cell lines as well as in primary patient samples in the in vitro assays, but not in normal cells.

In vitro Induction and Measurement of Apoptosis: Apoptosis is normal physiologic process which occurs during the embryonic development as well as in maintenance of tissue homeostasis. The apoptotic program is characterized by certain morphologic features, including loss of plasma membrane asymmetry and attachment condensation of cytoplasm and nucleus, and internucleosomal cleavage. Annexin V-FITC (BD Biosciences) is used to quantitatively determine the percentage of cells undergoing apoptosis. It relies on the property of cells to lose membrane asymmetry in the early phase of apoptosis. In apoptotic cells, the membrane phospholipids phosphatidylserine (PS) is translocated from the inner leaflet of the plasma membrane to the outer leaflet, thereby exposing PS to the external environment.

Annexin V is a calcium dependent phospholipids-binding protein that has a high affinity for PS, and is useful for identifying apoptotic cells with exposed PS. Propidium iodide (PI) is a standard flow cytometric viability probe and is used to distinguish viable from nonviable cells. Viable cells with intact membrane exclude PI, whereas the membranes of dead and damaged cells are permeable to PI. Cells that stain positive for Annexin V-FITC and negative for propidium iodide are undergoing apoptosis. Cells that stain positive for both Annexin V-FITC and PI are either in the end stage of apoptosis, undergoing necrosis, or are already dead. Cells that stain negative for both Annexin V-FITC and PI are alive and not undergoing measurable apoptosis.

For apoptotic assays, 1×10⁶ cells/well were seeded in 96-well flat bottom plates in RPMI media supplemented with of 10% fetal calf serum, 2 mM L-glutamine, 100 U/mL penicillin, 100 μg/mL streptomycin,at 37° C., 5% CO₂. The mAb 17.1 was added to the seeded cells in different concentration from 1 μg/mL up to 10 [g/mL in different wells. Since the mAb17.1 is mouse Isotype IgG₂b, commercial mouse IgG₂b from CALTAG was used as negative control at a concentration of 1 μg/mL up to 10 μg/mL in different wells. Commercial IgG₂b were dialysis to remove 01% sodium azide. For positive control, 10 μM camptothecin was added to the positive control culture wells. After 4 hrs of incubation in 37° C., 5% CO₂ labeled cells were washed 2 times with PBS to remove the excess antibodies. Re-suspended the cells in 100 μls of Annexin Binding Buffer (10 mM HEPES, pH 7.4, 140 mM NaCl, 2.5 mM CaCl₂) and added 5 μl of Annexin V-FITC and 10 μl of PI (50 μg/mL stock) to each samples. Plates were incubated for 20 minutes in room temp in the dark. After 20 minutes of incubation, cells were transfer to 5 ml FACS tube and 100 μl of 1×binding buffer was added to each tube for FACS analysis using FACS Aria. (Becton Dickinson). The FACS data were analyzed using FLOWJO.

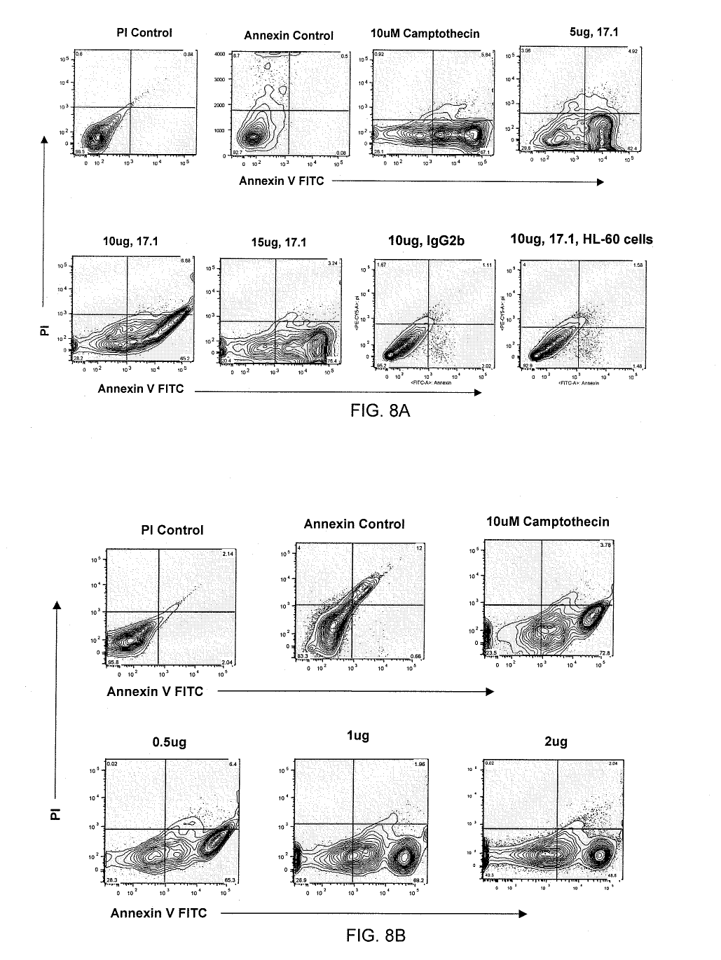

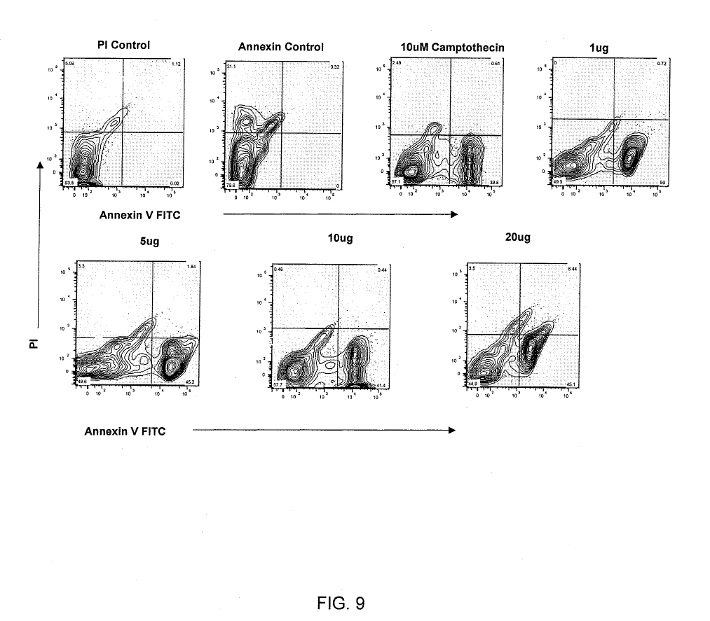

FIG. 8A show the induction of apoptosis with mAb 17.1 at 5, 10, and 15 μg in Kasumi-4 cells at 4 hrs. FIG. 8B show the induction of apoptosis with antibody 17.1 at 0.5, 1, and 2 μg in Kasumi-4 cells at 8 hrs. FIG. 9 show the induction of apoptosis with antibody 17.1 at 1, 5, 10, and 20 μg in a CML patient sample at 4 hrs. Table 6 is a summary of the induction of apoptosis with 17.1 mAb at 4 hours on Kasumi-4 cells, control HL-60 cells, and on normal PBMC (T Cells, B-Cells, NK Cells), shown as percent apoptosis.

TABLE 6

| 17.1 mAb | | | | | |
|---|---|---|---|---|---|
| (μg/ml) | Kasumi-4 | T Cells | B Cells | NK cells | HL-60 |
| 0.1 | 0.1% | 0.02% | 0.04% | 0 | 0.1% |
| 0.5 | 0.5% | 0.06% | 0.01% | 0.03% | 0.3% |
| 1 | 7.9% | 0.52% | 0.02% | 0.09% | 0.02% |
| 2 | 10.3% | 0.02% | 0.03% | 0.1% | 0.2% |
| 7 | 30.2% | 6.6% | 7.9% | 4.5% | 8.7% |
| 10 | 31.8% | 8.7% | 8.7% | 8.1% | 11.2% |

Induction of apoptosis with antibody 17.1 at 0.5, 1, 2, 5, 7 and 10 μg in a AML patient sample at 4 hrs (data not shown).

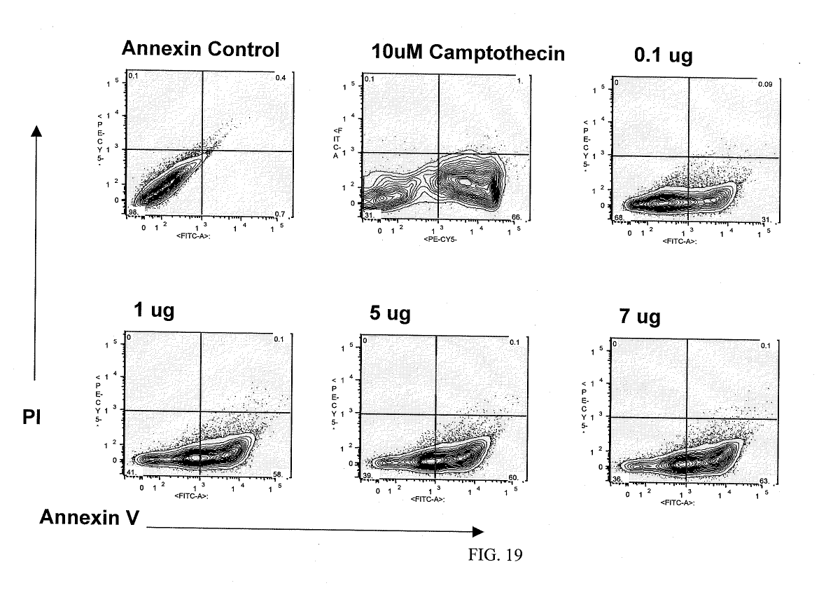

FIG. 19 shows the results of an experiment in which the 17.1 monoclonal antibody was assayed for its ability to induce apoptosis in the RL cell line. The 17.1 antibody is not immunoreactive with RL cells (data not shown). The antibody was provided at 0.1, 1, 5, and 7 μg.

Normal human peripheral blood mononuclear cells (PBMCs) were enriched by conventional methods and the 17.1 antibody was assayed for its ability to induce apoptosis in enriched populations of these cells. Table 6A is a summary of the induction of apoptosis with the 17.1 antibody at 4 hours on different cell populations enriched from normal human PBMCs in an apoptotic assay. Enrichment of PBMC cell populations was performed by conventional methods and the apoptotic assays were conducted as described above.

TABLE 6A

| 17.1 mAb | | | | |
|---|---|---|---|---|
| (μg/ml) | T Cells | B Cells | NK cells | HL-60 |
| 0.1 | 0.02 | 0.04 | 0 | 0.1 |
| 0.5 | 0.06 | 0.01 | 0.03 | 0.3 |
| 1 | 0.52 | 0.02 | 0.09 | 0.02 |
| 2 | 0.02 | 0.03 | 0.1 | 0.08 |
| 5 | 0.02 | 5.7 | 0 | 0.2 |
| 7 | 6.6 | 7.9 | 4.5 | 8.7 |
| 10 | 8.7 | 8.7 | 8.1 | 11.2 |

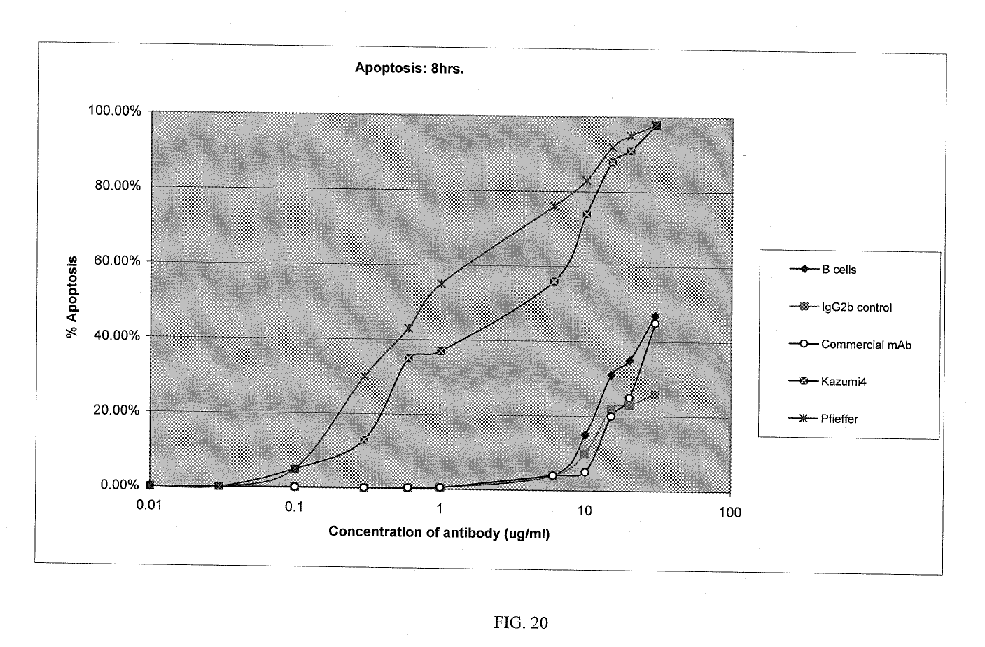

FIG. 20 shows the titration of the 17.1 monoclonal antibody in an apoptotic assay as the percentage of apoptosis at various concentrations of the antibody. The effect of the 17.1 monoclonal antibody was observed for the Pfeiffer cell line (ATCC CRL-2632), which is a model system for studying non-Hodgkin lymphomas, and the Kasumi-4 cell line, which is a human leukemia cell line established from the peripheral blood of a patient suffering from chronic myleogenous leukemia (CML). As a control, the 17.1 monoclonal antibody was assayed for its ability to induce apoptosis in B cells purified from normal human peripheral blood mononuclear cells (PBMCs). In another control experiment, mouse IgG₂b (IgG₂b isotype control—Caltag Laboratories, Burlingame, Calif., Catalog #:MG2b00) was assayed for its ability to induce apoptosis in the Pfeiffer cell line. Another CD45RA antibody (MEM-56) was also assayed for its ability to induce apoptosis in the Pfeiffer cell line.

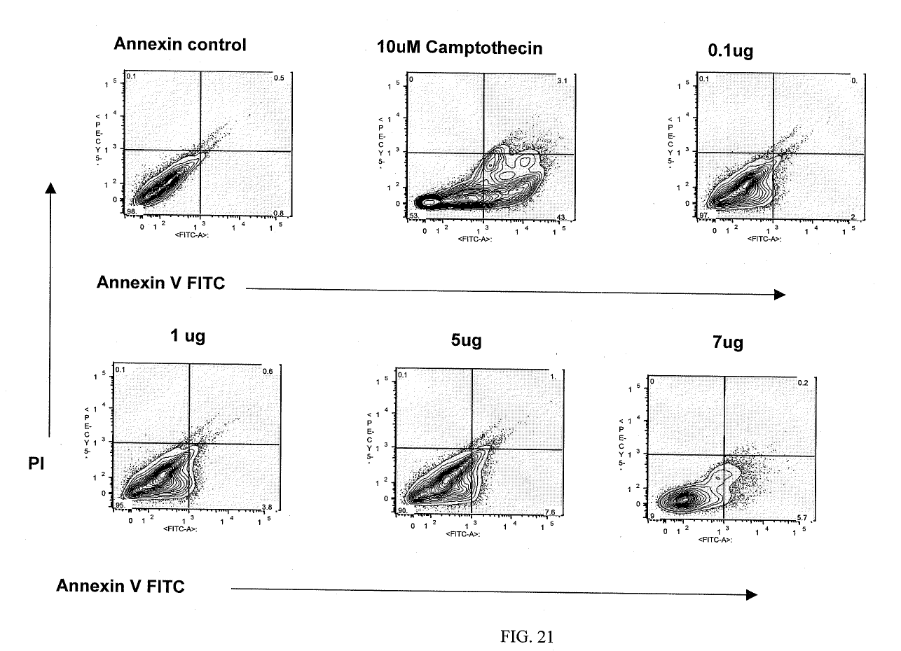

FIG. 21 shows the results of an experiment in which the 17.1 monoclonal antibody was assayed for its ability to induce apoptosis in the Molt-3 cell line, which is a T lymphoblast cell line derived from the peripheral blood of a patient with acute lymphoblastic leukemia (ALL). The antibody was provided at 0.1, 1, 5, and 7 μg.

Cellular growth and survival may be assayed according to the MTT assay (see Mosmann T. (1983) J. Immunol Methods. December 16;65(1-2):55-63, incorporated herein by reference in its entirety). MTT [3-(4,5-dimethylthiazol-2-yl)-2,5-diphenyltetrazolium bromide] is based on the ability of a mitochondrial dehydrogenase enzyme from viable cells to cleave the tetrazolium rings of the pale yellow MTT and form a dark blue formazan crystals which is largely impermeable to cell membranes, thus resulting in its accumulation within healthy cells. Solubilisation of the cells by the addition of a detergent results in the liberation of the crystals which are solubilized. The number of surviving cells is directly proportional to the level of the formazan product created. The color can then be quantified using a simple colorimetric assay. The results can be read on a multiwell scanning spectrophotometer (ELISA reader).

For an MTT assay, the Celltiter-96® Proliferation Assay from Promega (Cat.#G400) may be used. Test cells (Pfeiffer cells, KG-1A cells) and control cells (HL-60 cells, RL cells) can be adpated to 1%, 5%, and 10% FBS and maintained in log phase (˜1×10⁶ cells/mL). Cells can be seeded in 48-well plates for 24 hours with in 10% complete RPMI media (10% FCS). After 24 hours cells are washed with 1× PBS (to assure the binding of mAb to cell). The azide free 17.1 monoclonal antibody is added from 30 ug/ml to 0.01 ug/ml (30 ug, 20 ug, 15 ug, 10 ug, 6 ug, 3 ug, 1 ug, 0.3 ug, 0.01 ug). FACS can be performed to assure that mAb17.1 binds to the cell lines. Complete media is added. The negative controls may include 3 wells untreated with media only, 3 wells treated with IgG2B, and 3 wells with CD45RA (MEM-56 antibody). The positive control can include 5 uM and 10 uM JNK inhibitor SP600125 added to 3 wells. Cells are incubated at 37 degrees C. for 24, 48, 72, and 96 hours. Cells are monitored at different time points to observe cell death by microscope.

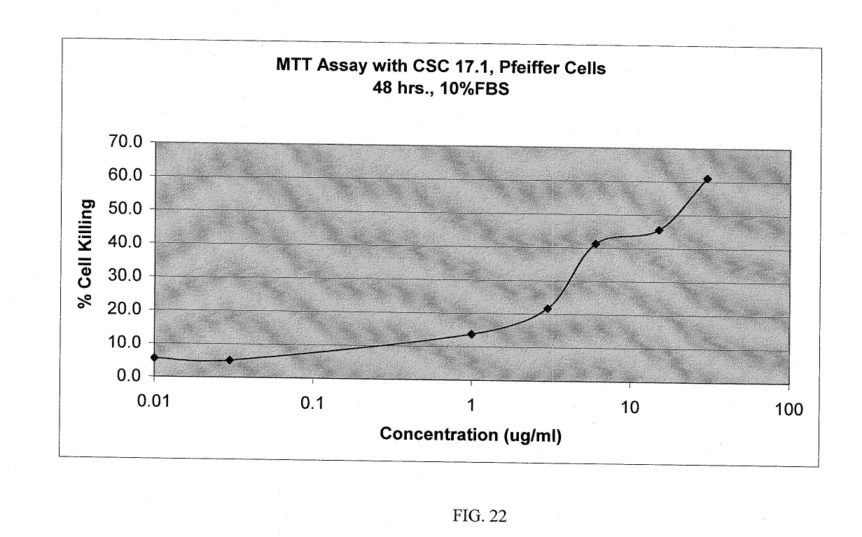

The MTT assay may be performed as follows. Pfeiffer cells were counted using Trypan Blue exclusion. 1.5×10⁴ cells/well were added to a 96-well flat bottom plate in 75 ul of media. 2× concentrations of IgG2b, antibody 17.1, and SP600125 were prepared in 75 ul of media and added to the appropriate wells. The plate was incubated at 37° C. for 24, 48, 72, and 96 hours. After each time point is complete, 22.5 ul of MTT Solution was added to each well and incubated at 37° C. for 4 hours. 150 ul of the Solubilization/Stop solution was then added to each well and incubated for one hour. The contents of each well is mixed to get a uniform color but bubble formation is avoided. Absorbance is recorded at 570 nm using a 96-well plate reader. In addition, the absorbance is recorded at a reference wavelength of 650 nm or above. This will reduce the background contributed by cell debris, fingerprints, or other non-specific absorbance. An MTT assay was performed using Pfeiffer cells and after 48 hours, the results were observed. FIG. 22 shows the results of an MTT assay of Pfeiffer cells treated with the 17.1 antibody for 48 hours in 10% FBS.

The inhibitory effect by the 17.1 antibody on the growth of tumor cells may be observed from counting the number of cells. Cells may be counted as is generally known in the art, such as through the use of a hemocytometer. In addition, the inhibition of tumor cell growth may be observed visually by treatment of cells with the 17.1 antibody in multi-well plates as is generally known in the art.

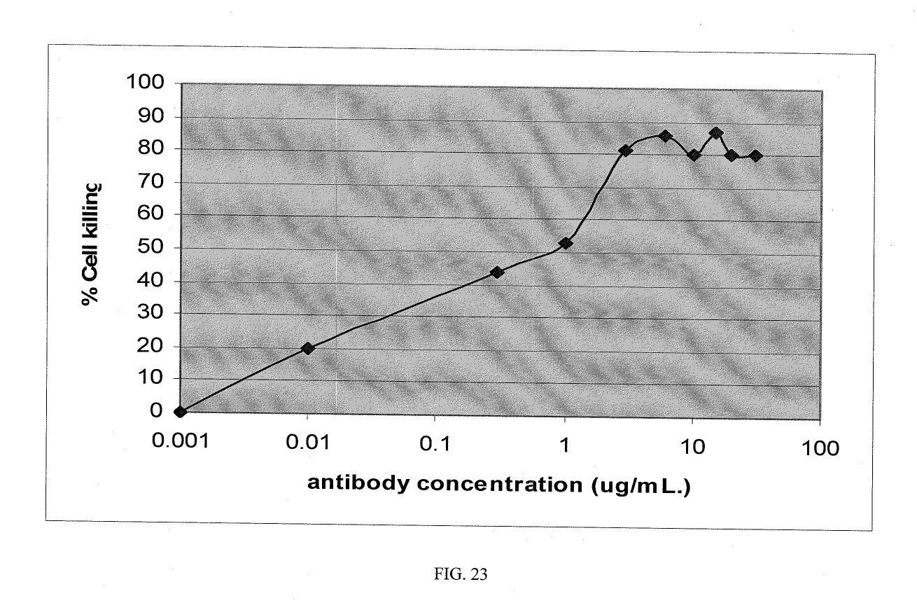

FIG. 23 shows the results of a cell count performed following incubation of the 17.1 antibody with Pfeiffer cells at the various concentrations indicated. The cell count was performed by incubating Pfeiffer cells in 48 well plates for 48 hours with different concentration of antibody. The viable cells were then counted by trypan blue exclusion. There was 100% viability in medium only (no antibody) and then toxicity was calculated (% cell killing).

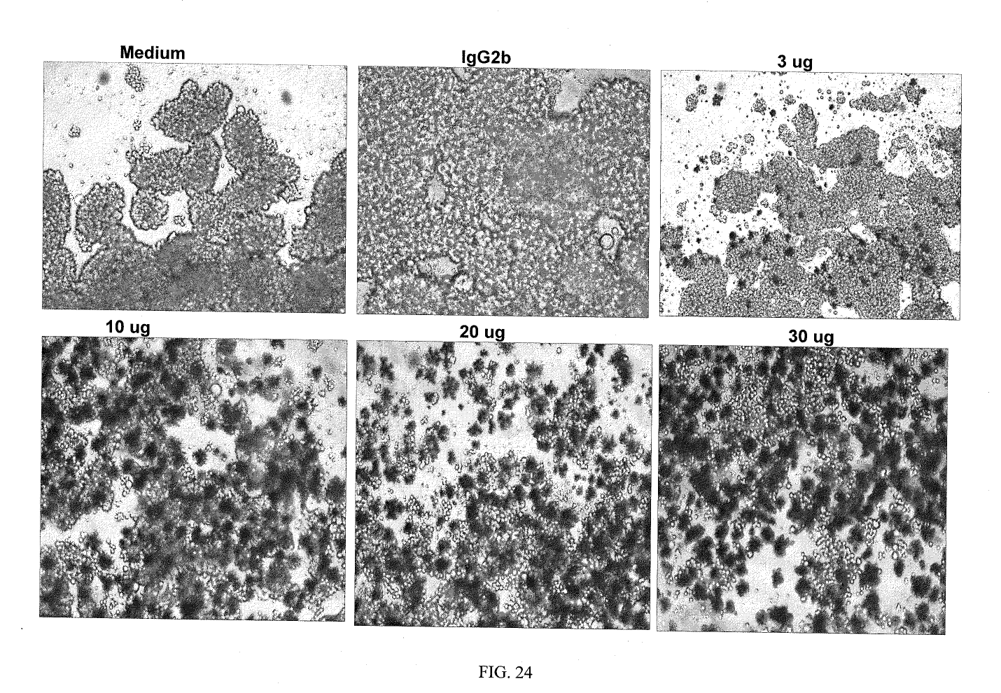

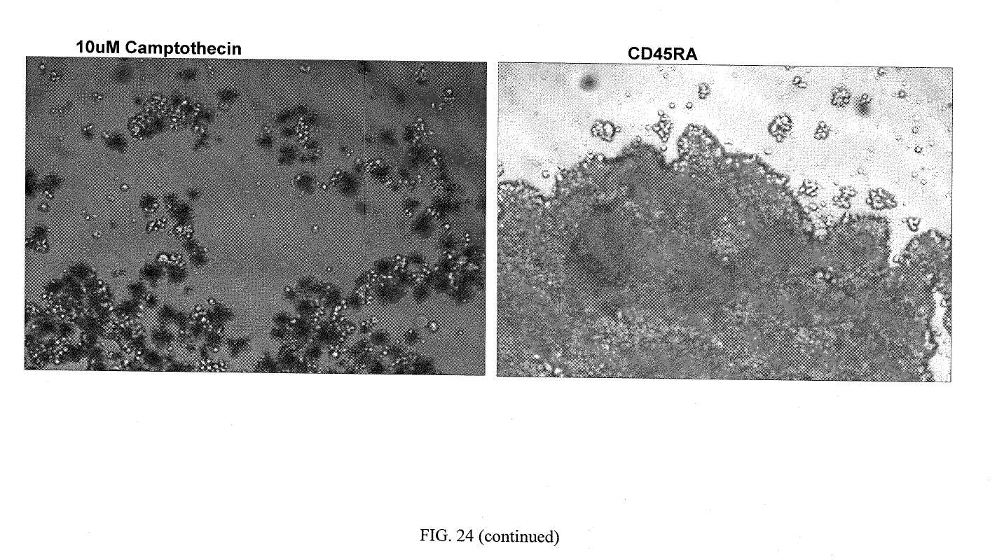

FIG. 24 shows the degree to which the 17.1 antibody inhibits the growth of Pfeiffer cells in multi-well plates at various concentrations (3 ug, 10 ug, 20 ug, and 30 ug). Camptothecin was used as a positive control. Medium-treated cells, the CD45RA antibody (MEM-56), and IgG2b were used as negative controls.

### 5.6.1 Example 6A

### Determination of the Internalization of the 17.1 Monoclonal Antibody

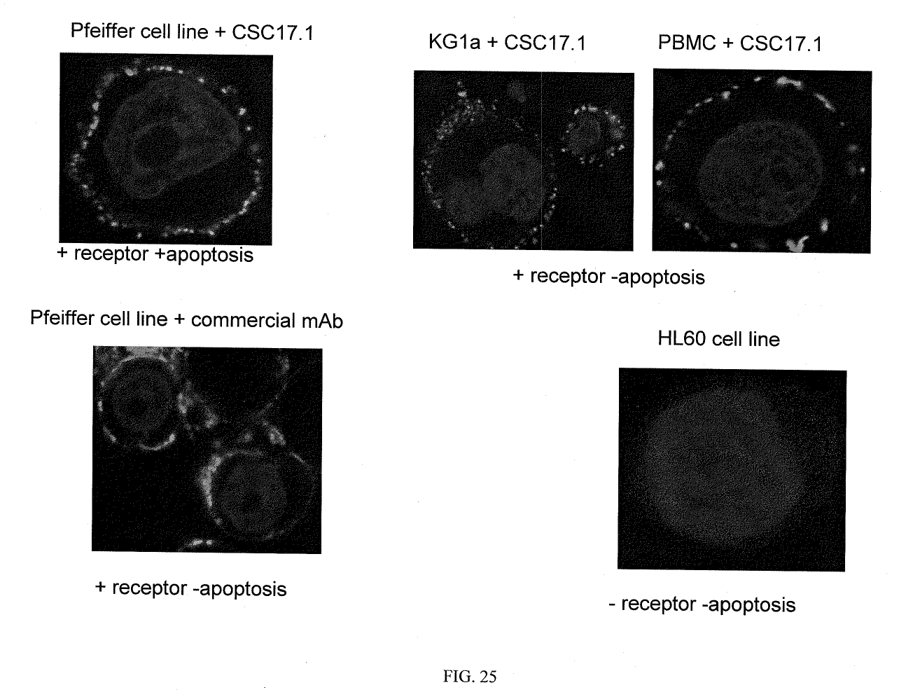

Confocal microscopy was used to determine the degree of internalization of the 17.1 mAb following binding to cell surface receptors. The following protocol for confocal microscopy staining was used. The cells used were the Pfeiffer cell line, the KG1a cell line, PBMC, and the HL60 cell line. Cells were fixed in 1.5% paraformaldehyde in 15 mL. tubes, 2×10⁶ cells per tube for 20 minutes at room temperature. Then the cells were spun down, the supernatant decanted and the cells resuspended in a small volume of staining medium. 1 ml of ice cold methanol was added to the cells and they were resuspended (1 mL. methanol per 2-5×10⁶ cells) to permeabilize for 10 minutes at 4° C. over night at −20° C. or longer at −80° C. Then 1 mL of staining medium per mL of methanol was added and the cells were spun down and then washed once in staining medium. The cells were then resuspended at 1×10⁶cells per 100 to 200 uls of staining medium. To the cells is added CSC17.1 (1 ug/mL.) for 40 minutes at 37° C. Then the cells were washed and secondary antibody Goat anti-mouse Alexa 488 was added and incubated for 15 minutes at 37° C. Staining medium was added, the cells were washed, transferred to glass slides, and glass coverslips were placed for confocal microscopy. FIG. 25 shows the results of the confocal microscopy in which the 17.1 antibody was found not to internalize upon binding to cell surface receptors.

### 5.7 Example 7

### In Vivo Efficacy of Monoclonal Antibodies Against Cancer

In vivo models of human cancer are useful to determine preclinical efficacy of candidate therapeutic agents. For monoclonal antibodies, studies in appropriate animal models help evaluate target cell lysis and tumor eradication under physiological conditions in vivo. Several groups have described engraftment of CML chronic phase (CP), accelerated phase (AP), and/or blast phase (BP) and AML cells into SCID and NOD/SCID mice. In general, generation of chimeric animals showing engraftment of human CML cells is more consistent in NOD/SCID mice (See Dazzi, F et al, Blood 92: 1390-1396 (1998); Wang, J. C. Y. et al, Blood 91: 2406-2414 (1998); Dick, J et al Blood 87: 1539-1548 (1996); Bonnet, D et al, Blood 106: 4086-4092 (2005)). In vivo efficacy of monoclonal antibodies against CML and/or normal GMP and not HSC can be determined using the NOD/SCID human CML model.

Xenograft animals can be generated as described by Dazzi et al. Briefly, NOD/SCID mice are bred in house or purchased from a commercial supplier (Jackson Laboratories) and housed under pathogen-free conditions. Prior to injection of cells, animals are irradiated (250 cGy, x-ray source). Cryopreserved cells from a CML or AML patient obtained from peripheral blood, mobilized peripheral blood or bone marrow using are analyzed by flow cytometry to determine the percentage of CD34⁺ cells in the sample. Samples containing 1 to 10×10⁶ CD34⁺ cells are injected IV into the conditioned mouse in a total volume of 1 mL. Alternatively, CD34⁺ cells can be sorted from the sample by FACS prior to transplantation. A subset of the animals are sacrificed weekly and bone marrow and spleen analyzed for human CD34⁺ cells. Patient samples with engraftment potential are selected for use in antibody efficacy studies. For efficacy studies, CML/AML cells are transplanted and the test monoclonal antibody or control antibody will be injected on a schedule. Alternate schedules include once to 3 times per week, 1-3 injections per week for 1-4 weeks, or 1-2 per week for 1-4 months. Injections can be intravenous by tail vein injection, intraperitoneal, subcutaneous, or intramuscular. Following completion of the treatment schedule, animals are sacrificed and tissues collected for analysis. Peripheral blood, spleen and bone marrow can be evaluated by FACS analysis for the presence of human phenotypic CML cancer stem cells, CD45LCA⁺ CD34⁺ CD45RA⁺, detectable in the bone marrow and spleen at the conclusion of the treatment. Philadelphia (Ph) chromosome can be assayed by PCR to determine whether the cells are CML or normal.

Eleven mice were transplanted with CML sample (MISIRB 31104 750), 5×10⁶ cells/mouse. Mice were conditioned with 250rad TBI (x-ray source, Faxitron CP160), and anti-asialo GM1. The anti-asialo GM1 is injected by intraperitoneal injection on days 0, 5 and 11. At 4 weeks post transplant half the mice in each group will begin receiving intraperitoneal or intravenous injections of clone 17 or control antibody, 250-1000 mg/dose, two times a week for 4 weeks. Additionally, a group of 40 mice are also transplanted with CML (MISIRB 31104 750) as above. Antibody administration begins at the time of transplant. Mice are injected by intraperitoneal injection with 0.5-1 mg/dose of antibody, twice a week for 8 weeks. Alternatively, CML cells isolated from previously engrafted mice will be serially transplanted. Some of these secondary recipients will be treated with clone 17 at time of transplantation by intraperitoneal injection with 0.5-1 mg/dose of antibody, twice a week for 8 weeks. Following the above treatments with clone 17 the mice are analyzed by flow cytometry for tumor burden, expression of CD45LCA, CD34 and CD45RA. The number and frequency of human cells in the bone marrow and spleen will be determined for all mice surviving to the end of the study. Human cells (CD45LCA+) will be sorted by FACS from both groups of mice for serial transplantation to determine if cells with functional cancer stem cell potential are present.

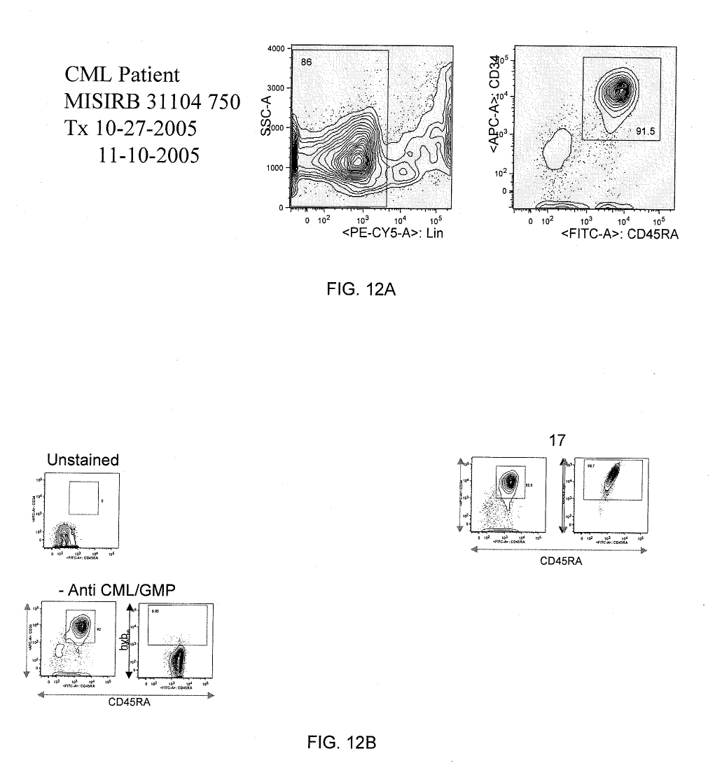

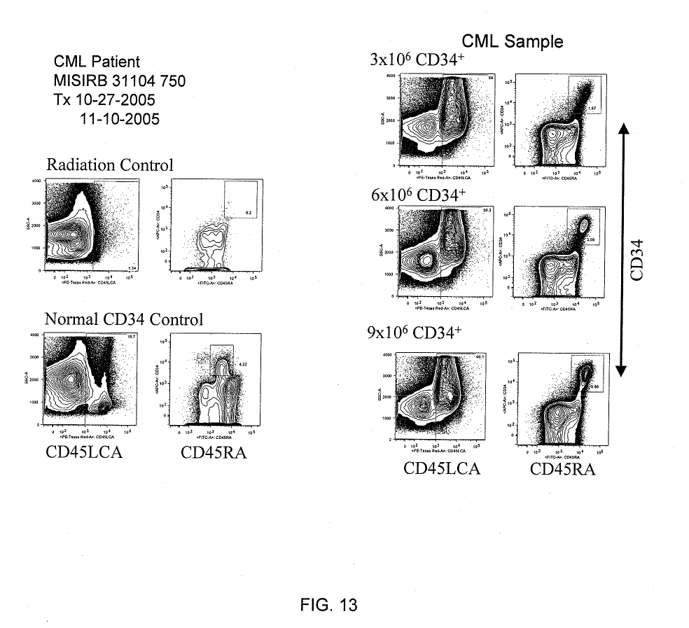

FIG. 12A shows the CML peripheral blood sample-blast crisis (MISIRB 31104 750) in the transplantation of CML in NOD/SCID mouse model. FIG. 12B shows the binding of mAbs 17.1 to CML peripheral blood sample. FIG. 13 is NOD/SCID analysis of CD34 compartment, bone marrow, 11 weeks post transplant. NOD/SCID analysis of CD34 compartment, spleen, 11 weeks post transplant (data not shown).

For secondary transplant of CML cells CD34⁺CD45RA⁺ cells were sorted from the bone marrow and spleen of several mice for transplantation (FIG. 14). FIG. 15 shows the NOD/SCID analysis, secondary transplant, 10 weeks post transplant with the CD34 compartment of bone marrow (15A) and spleen (15B).

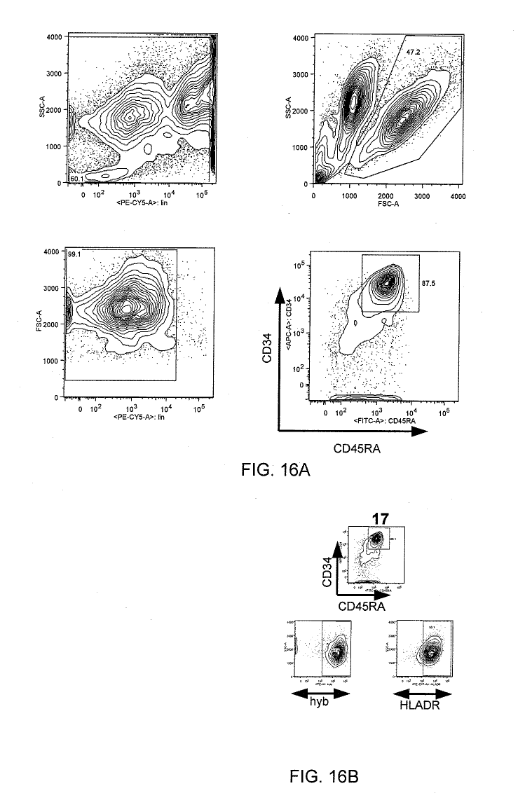

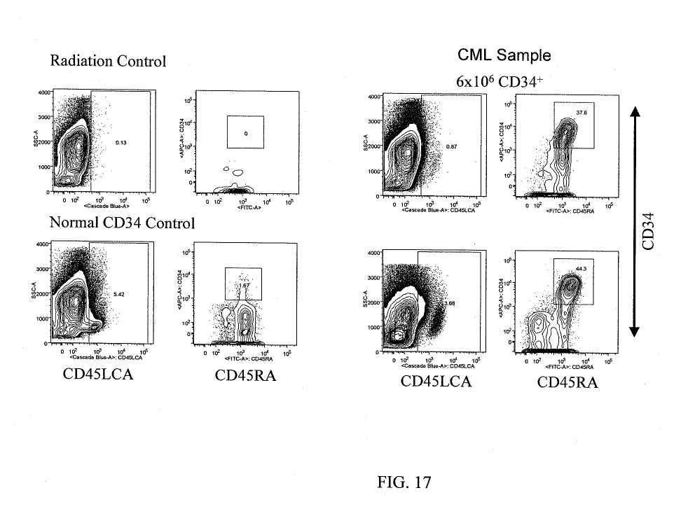

A second sample of CML peripheral blood sample-blast crisis, patient sample (LATLi 493 20030108 CML) (FIG. 16A). FIG. 16B shows the binding of mAbs 17.1 to CML peripheral blood sample. FIG. 17 is NOD/SCID analysis of CD34 compartment, bone marrow, 8 weeks post transplant.

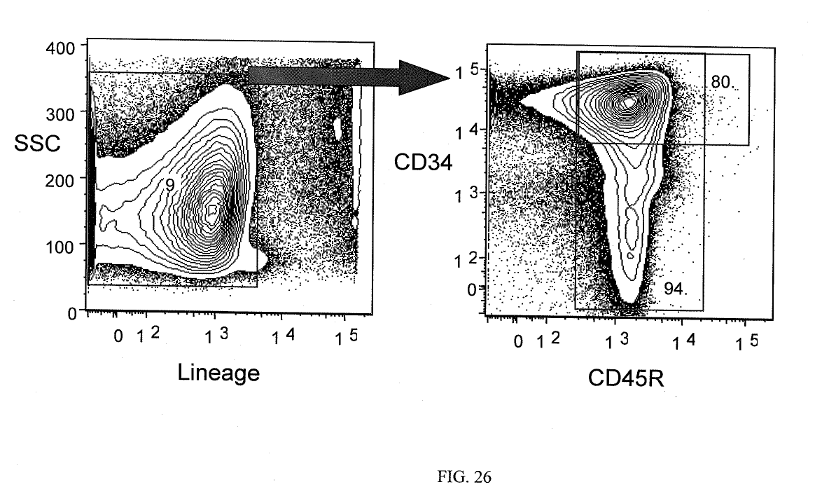

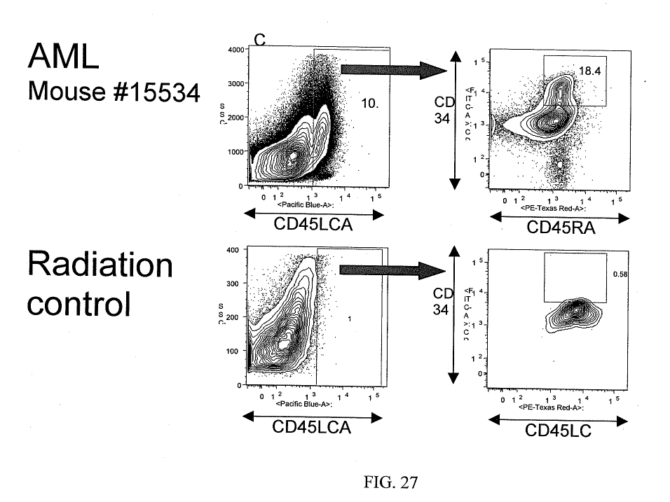

Example of the AML in vivo model. FIG. 26 illustrates the expression of CD45RA on AML patient UOV12-38 blood sample by FACS. Greater than 90% of the cells in the sample express CD45RA, 80% express both CD45RA and CD34. FIG. 27 shows analysis of a NOD/SCID mouse transplanted with AML patient sample UOV12-38. CD45LCA⁺ human cells are present expressing CD45RA and CD34.

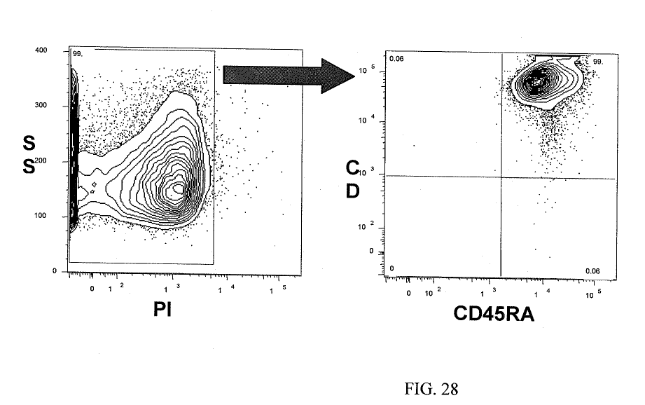

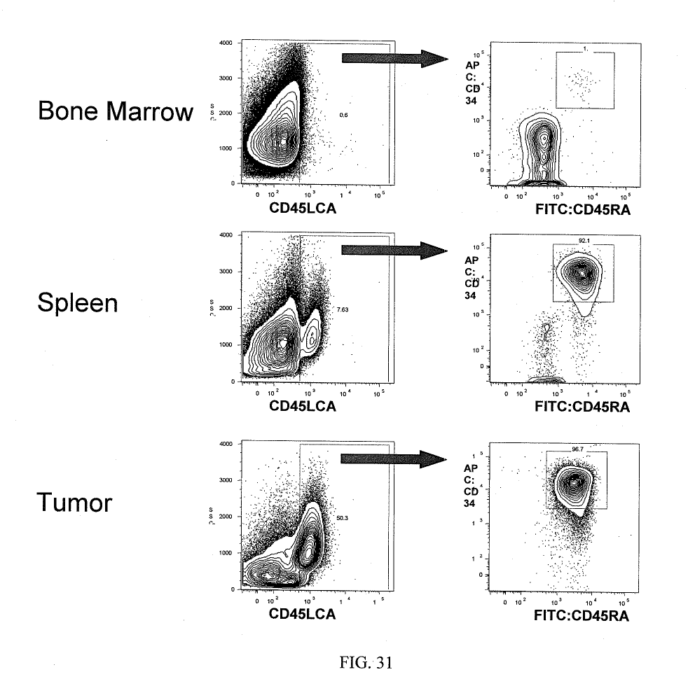

Example of cell line in vivo model. FIG. 28 shows expression of CD45RA by the cell line KG-1a. Analysis of mice transplanted subcutaneously with KG-1a cells (FIG. 31) demonstrates that the engrafting human cells maintain expression of CD45RA. Human CD45RA⁺ cells are detected at the site of injection (tumor), the spleen and bone marrow.

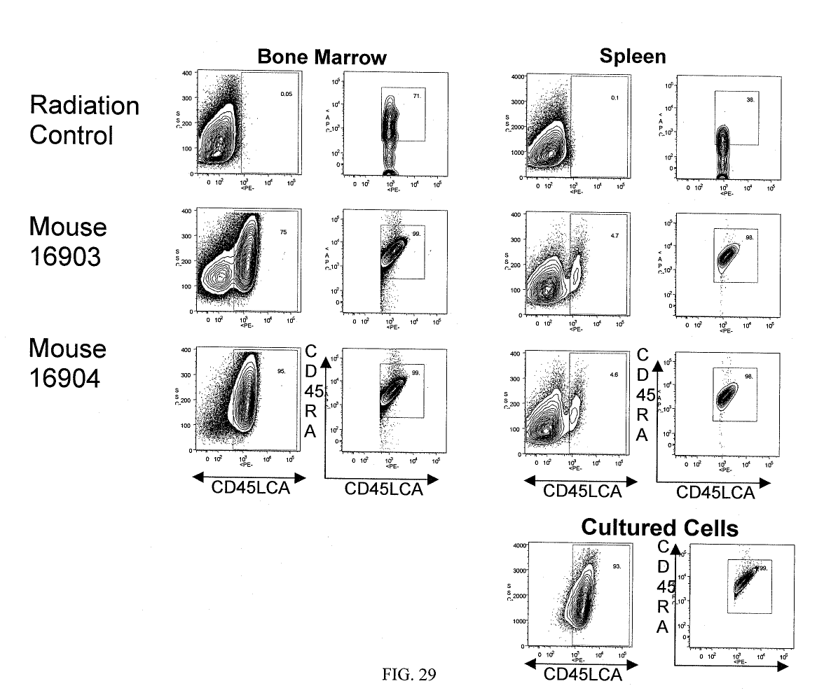

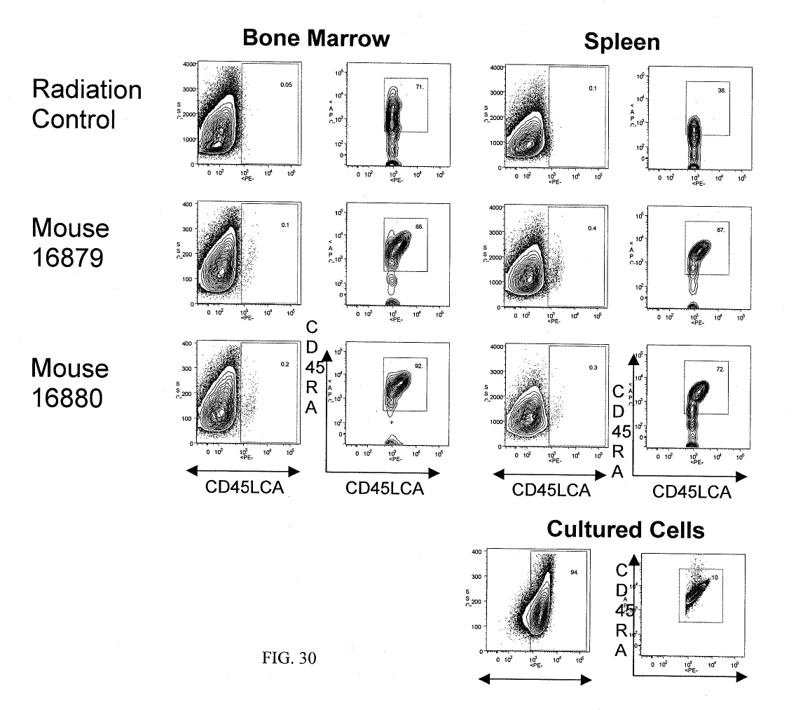

Example of cell line in vivo model. FIG. 29 and FIG. 30 shows expression of CD45RA by the Burkift lymphoma cell lines GA-10 and Ramos, respectively. Each experiment includes a two week analysis of 1×10⁷ cells injected intravenously. Analysis of mice transplanted by intravenous injection with GA-10 and Ramos cells shows that the engrafting human cells maintain expression of CD45RA. Human CD45RA⁺ cells are detectable in the spleen and bone marrow.

To determine if the monoclonal antibody clone 17.1 can eliminate or reduce tumor burden in mice transplanted with primary human AML blast crisis cells. Twenty-five mice were transplanted with AML cells injected at 10×10⁶ CD34⁺CD45RA⁺ cells/mouse (AML sample UOV12-38). Cells are injected intravenously into the tail vein or the post lateral aspect of the orbital cavity. Mice are conditioned with 250rad TBI (x-ray source, Faxitron CP160), and anti-asialo GM1. The anti-asialo GM1 is injected IP on days 0, 5 and 11. Beginning 4 weeks post transplant, mice are randomized into 2 groups, therapy and control. The group receiving the therapeutic is injected I.P. with clone 17.1. Antibody will be administered by intraperitoneal injection 2 times a week, for 4 weeks. Antibody concentration will be 1 mg per injection (a total of 2 mg/mouse/week). Volume will vary depending on the antibody lot used. Mouse IgG will be used as the control article; it will be prepared in the same diluent and injected at the same concentration and volume as the monoclonal antibody. Mice will be sacrificed 2-3 days following the last injection of clone 17.1. Bone marrow and spleen will be isolated and counted. Tissues will be analyzed by FACS for expression of CD45LCA, CD34 & CD45RA. The number and frequency of human cells in the bone marrow and spleen will be determined for all mice surviving to the end of the study. Human cells will be sorted by FACS from both groups of mice for serial transplantation to determine if cells with functional cancer stem cell potential are present post antibody treatment. Alternatively, mice will begin antibody treatment at the time of AML cell transplant. These mice will be injected with 0.5 mg of antibody 2 times a week for 8 weeks. Analysis will proceed as described above.

In addition, the efficacy of clone 17.1 will be tested for efficacy in leukemia and lymphoma in vivo models using human cell lines expressing CD45RA. Immunocompromised animals will be inoculated with human leukemia or lymphoma cell lines recognized by clone 17.1. The efficacy of clone 17.1 will be tested using multiple cell lines. The cell lines used should maintain a primitive phenotype in vivo with sustained expression of the epitope recognized by clone 17.1. Examples of leukemia or lymphoma cell lines expressing CD45RA are KG-1a, Pfeiffer, Ramos, U-937, HEL, GA-10, THP1, AML193, Kasumi-4 and Molt. Additional cells lines will be identified and used as appropriate. Each cell line will be characterized using different routes of administration; intravenous, subcutaneous or intraperitoneal injection. NOD/SCID mice will be tested for tumor engraftment at the site of injection and subsequent invasion of bone marrow and spleen. Animals will be treated with clone 17.1 beginning at the time of tumor inoculation or following tumor engraftment with injections starting 1-4 weeks post cell administration. Antibody will be administered by intraperitoneal injection 2 times a week, for 4 weeks. Mouse IgG will be used as the control article, injected at the same concentration and volume as clone 17.1. Test cells will be administered at a single site. Animals are weighed weekly and observed for clinical signs of toxicity and death for the duration of treatment. Animals are observed and palpated for the formation of nodules at the site of injection twice weekly, detected nodules will be measured in two dimensions and findings recorded. The injection site is exposed at the end of study and tumor removed and measured and weighed. In addition, a sample of bone marrow, spleen and tumor mass are to be removed for phenotyping by FACS. These tissues will be disassociated and prepared for analysis by flow cytometry. Tissues will be screened for expression of the human pan-leukocyte marker CD45LCA and clone 17.1.

### 5.8 Example 8

### Determination of Binding/Lytic Activity of Monoclonal Antibodies on CML/AML and not HSC

Jamieson et al. The New England Journal of Medicine 351: 657-667 ( 2004), suggests the CML cancer stem cell shares a phenotype with the GMP. The monoclonal antibodies disclosed herein bind GMP cells (See Table 1). The lack of or minimal binding of the monoclonal antibodies to the HSC will allow effective elimination of normal GMP and CML stem cells for cancer treatment but allow endogenous reconstitution of the host hematopoietic system. Assays to determine the extent of HSC binding or non-binding of disclosed monoclonal antibodies that target GMP or CML stem cells are described below.

Normal human HSC are purified from cord blood, bone marrow, or GCSF mobilized peripheral blood and transplanted into NOD/SCID mice as previously reported by several investigators. See Dazzi, F et al, Wang, J. C. Y. et al; Dick, J et al. Briefly, NOD/SCID mice are exposed to 250cGy using a Cs source irradiator. Mice are injected intravenously with 0.1 to 5×10⁶ purified human HSC (CD34⁺ CD90⁺) that have been previously treated in vitro to a monoclonal antibodies disclosed herein and complement. Non-specific, isotype matched antibody and complement or complement alone is used as the control treatment. A monoclonal antibody that binds human CD34⁺ may also be used as a positive control for HSC killing in vitro. Kinetics of human cell chimerism can be determined by FACS analysis using an anti-human CD45 antibody. Alternatively, selected animals are sacrificed and the percent human cells in the bone marrow and spleen can be determined.

### 5.10 Example 9

### Immunoprecipitation of Cell Lysates with Monoclonal Antibodies

Immunoprecipitation: Cell surface antigens recognized by the monoclonal antibodies can be isolated and identified by immunoprecipitation from cell lysates. Cell surface proteins of human acute myeloid leukemia KG1a cells were biotinylated and the cells washed three times in PBS. Cells were lysed in 25 mM Phosphate pH 7.5, 50 mM NaCl, 1% CHAPS, 1 mM EDTA, and protease inhibitors (Calbiochem). The cell lysate was immunoprecipitated with 10 μg antibody followed by immobilization onto protein G sepharose beads. The beads were washed, boiled and loaded on a 4-12% Bis-Tris gel (Invitrogen). The gel was then electroblotted onto PVDF membrane, blocked with 5% BSA and washed with PBS/0.05% Tween (PBST). The membrane was then incubated with Neutravidin-HRP (Pierce), washed with PBST, and developed with Pierce ECL chemiluminescence substrate and detected with an Alpha Innotech imager. For silver stained immunoprecipitation, KG1a cells were not biotinylated or electroblotted, instead the gels were stained using the SilverQuest staining kit (Invitrogen). Antigens were identified by excising the proper band on the silver stained gel and submitted for mass spectrometry/mass mapping studies.

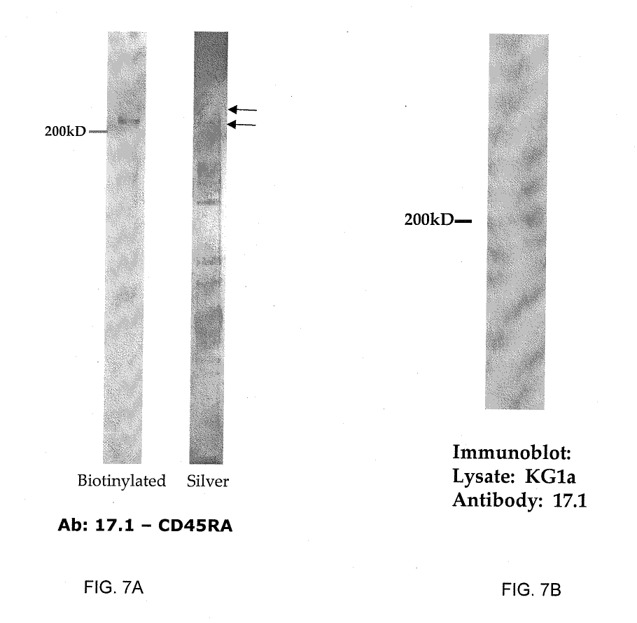

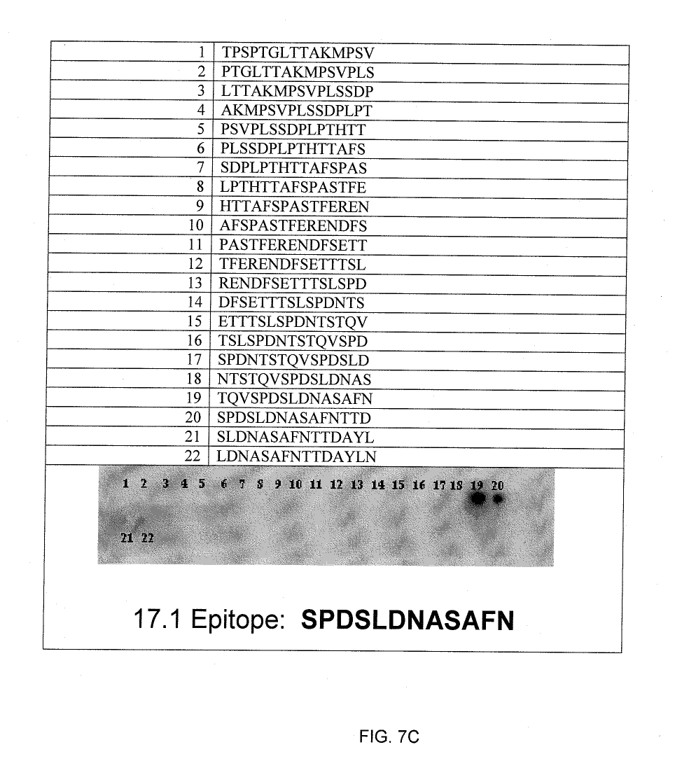

FIG. 7A shows the results of the immunoprecipitation of KG1a cell lysate with antibody 17.1. Immunoprecipitation using the biotinylated KG1a cell-lysate sample precipitated a roughly 200 kD antigen (left blot). The silver stained immunoprecipitation (right blot) showed two high molecular weight species that have similar molecular weight as the biotinylated sample. These two bands were excised and analyzed by MALDI/mass mapping studies which identified both bands to be protein tyrosine phosphatase, receptor type, C (CD45). Again the biotinylated immunoprecipitation (left blot) shows two set of doublets one at approximately 120 kD and another at 35 kD. The corresponding bands on the silver stained immunoprecipitation gel were excised and analyzed by MALDI/mass mapping studies. The higher molecular weight bands were identified as immunoglobulins, likely from residual antibodies. The lower molecular weight doublets (*) were identified as HLA-DR (major histocompatibility complex, class II, DR).

Western Blot: KG1a cells were lysed in 25 mM Tris pH 7.5, 1% CHAPS, 50 mM NaCl, 1 mM EDTA, and protease inhibitors. Approximately 25 ug of lysate per lane was loaded on a Bis-Tris gel. Gel was run at 100V and electroblotted onto PVDF. Membrane was blocked with 5% BSA and washed before incubation with 10 ug/ml of antibody 17.1. After washing, the membrane incubated in HRP-conjugated goat anti-mouse IgG secondary antibody and detected with Pierce ECL chemiluninescence kit. Gel image was recorded by bioluminescence imager (Innotech)

FIG. 7B shows that KG1a cells express the antigen to monoclonal antibody 17.1. At approximately 200 kD, the protein is a higher molecular weight protein.

Identification of the target antigen of mAb 17.1 by immunoprecipitation followed by mass mapping: The surface antigen recognized by a monoclonal antibody can be isolated by immunoprecipitation and/or immunoaffinity purification from cell lysates. Both methods require the binding of the target antigen to the monoclonal antibody followed by the immobilization of this antibody-antigen complex to a solid-phase matrix like protein G sepharose. After washing the reaction to remove any binding of non-specific proteins, the target protein is then eluted from the antibody-coupled sepharose beads. The eluted protein may be analyzed by gel electrophoresis to determine molecular weight. The same electrophoretic bands can used to determine the identity of the protein by mass mapping. Here, the protein (gel band) is cut out of the gel and is digested with a specific enzyme (e.g. trypsin) under controlled conditions. After purification by HPLC (High Performance Liquid Chromatography), the mixture protein fragments are analyzed using mass spectrometry techniques like MALDI (Matrix-Assisted Laser Desorption Ionization). Once the mass measurements have been acquired, they are compared against various databases that contain masses of proteolytic peptides expected from enzymatic digestion of proteins of known sequences to determine the identity. Database comparison is performed through the use of license search algorithm software like Mascot (www.matrixscience.com), Sequest (www.enovatia.com), or Protein Prospector (http://prospector.ucsf.edu).

Epitope mapping of monoclonal antibody 17.1: Partially overlapping 15 amino acid peptides with a three amino acid shift spanning the sequence of exon 4 of CD45 were synthesized on cellulose membrane supported by the C-terminus. The membrane was rinsed in methanol, washed with Tris Buffer Saline (TBS) and blocked with 5% BSA before incubation with 10 ug/ml of monoclonal antibody 17.1 for three hours. After washing, the bound antibodies were detected with HRP-conjugated goat anti-mouse IgG follow by a chemiluminescence substrate. The result was capture by a chemiluminescene imager (Innotech). The membrane was regenerated by washing with 62.5 mM Tris pH 6.7, 2% SDS, 100 mM 2-mercaptoethanol and tested by incubation of the membrane with conjugate alone before blotting with isotype IgG control primary antibodies.

FIG. 7C shows epitope mapping of mAb 17.1. The table shows overlapping peptide sequences (1-22). The blot of the overlapping peptides sequence with monoclonal antibody 17.1 resulted in positive staining for spots 19 and 20. The overlapping amino acid sequence for these two spots (SPDSLDNASAFN) represents the binding epitope for antibody 17.1.

### 5.11 Example 10

### Immunocytochemistry on Frozen Human Multi-Tissue Array with Monoclonal Antibodies

Monoclonal antibody 17.1 was used to test reactivity with various human tissues. Immunohistochemistry results were obtained using the Dako Envision system as described below.

The reagents used in the experiment include a Wash Buffer (Dako Cytomation, Code#S3006, Lot#00002847, Exp.07/07), an Antibody Diluent (Dako Cytomation, Code# S0809, Lot# 00003303, Exp. 02/08), a Dako Peroxidase Block (Dako Cytomation, Code#K4007, Lot#00002809, Exp.07/08), a Detection System (Envision+ System, HRP (DAB) Mouse)—Dako Cytomation, Code#K4007, Lot#00002809 , Exp. 07/08)), a Counterstain (Hematoxylin (Richard Allen, Code#7211, Lot#56626, Exp. 02/07)), a Bluing Reagent (Richard Allen, Code#7301, Lot#52639, Exp#11/07), and a Mounting Medium (Richard Allen, Code#4111, Lot# 43240, Exp. 03/07).

The following protocol was used. Clone 17.1 Antibody was diluted in the Antibody Diluent. Sections were covered with Peroxidase Block for 5 minutes at room temperature followed by rinsing with Wash Buffer 2×5 minutes without agitation. Approximately 200 μl (making sure section is covered) of Clone 17.1 dilution is added and incubated for 60 minutes at room temperature and then rinsed in Wash Buffer 2×5 minutes. Then approximately 200 μl of Secondary Antibody from Detection System is added and incubated for 30 minutes at room temperature followed by rinsing in Wash Buffer 2×5 minutes. Chromogen (DAB+ provided in Detection System) is added to cover the entire section (˜200 μl) and incubated for 5 minutes. Slides were checked for proper development. Positive staining was indicated by a dark brown chromogen. The slides were rinsed in distilled water for 5 minutes without agitation and then counterstained with Hematoxylin for 30 seconds. Hematoxylin provides a blue nuclear stain. Then the slides are rinsed in distilled water for 5 minutes, dipped twice in Bluing Reagent and rinsed again in distilled water for 5 minutes. The slides are dehydrated in 95% Ethanol 2×2 minutes, 100% Ethanol 2×2 minutes. The slides are cleared in Xylene and then the Mounting Medium and coverslip are added.

Results of the binding of the 17.1 antibody to on frozen human multi-tissue array with 17.1 is compiled in Table 7. The results were scored as “Positive” for positive staining and “Negative” for negative staining.

TABLE 7

| Tissue Type | 17.1 |
|---|---|
| Colon | Negative |
| Esophagus | Negative |
| Ileum | Negative |
| Stomach | Negative |
| Salivary gland | Negative |
| Pancreas | Negative |
| Liver | Negative |
| Lung | Negative |
| Breast | Glandular epithelium, 3 Positive |
| Breast | Adipose tissue only, Negative |
| Ovary | Negative |
| Endometrium | Scattered lymphocytes, 3 Positive |
| Endometrium | Glandular epithelium, 3 Positive |
| Prostate | Negative |
| Testis | Negative |
| Kidney | Negative |
| Urinary bladder | Negative |
| Adrenal gland | Negative |
| Thyroid gland | Negative |
| Lymph node | Lymphocytes, 3 Positive |
| Lymph node | Adipose tissue only, Negative |
| Thymus | Lymphocytes, 3 Positive |
| Thymus | Adipose tissue only, Negative |
| Spleen | White pulp, 3 Positive |

### 5.12 Example 11

### Apoptosis of KG1a Cells Incubated with Antibody 17.1 and Antibody MEM-56, a Monoclonal to Human CD45RA, With and Without Crosslinkers

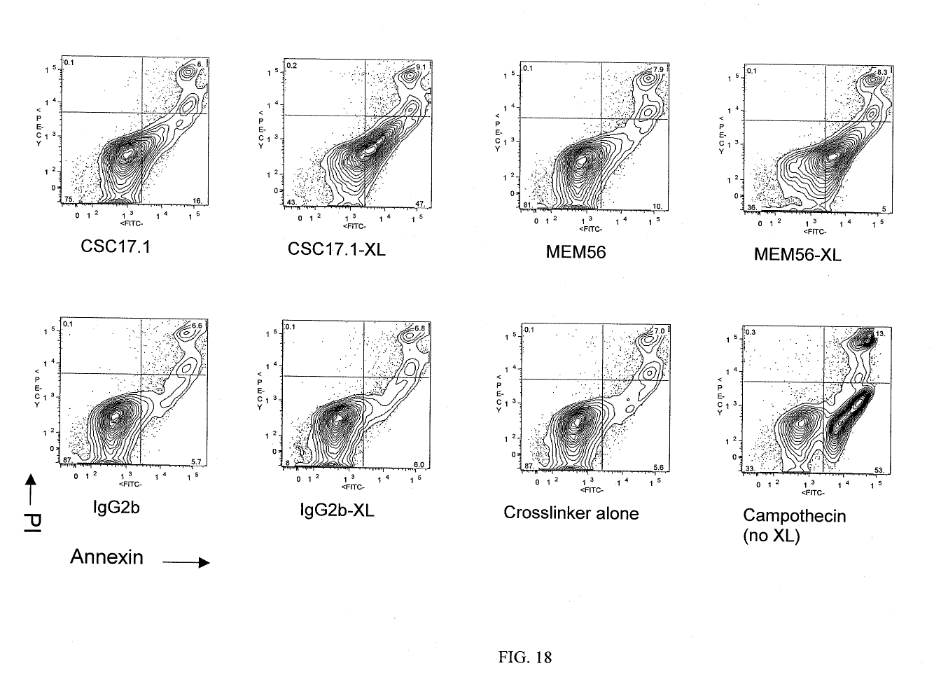

KG1a cells were incubated for 24 hours with antibody alone or antibody in the presence of GAM (goat anti-mouse antibody) crosslinker. Cell were then stained with annexin V-FITC and analyzed by flow cytometry. FIG. 18 shows apoptosis of KG1a cells incubated with antibody 17.1 and antibody MEM-56, a monoclonal to human CD45RA, with and without crosslinkers. 17.1 by itself induced apoptosis and this level of apoptosis increase two-fold with the addition of a crosslinker. The anti-CD45RA antibody MEM-56 did not cause apoptosis when added alone, however, in the presence of the goat anti-mouse crosslinker, the level of apoptosis increased five fold compared to antibody alone. The crosslinker did not change the negative apoptosis level of the IgG2b isotype control and also by did not cause any apoptosis by itself.

### 5.13 Example 12

### Blocking of Antibody 17.1 by CD45RA Antibodies

A blocking experiment was performed to measure any loss of binding capability of CSC17.1 to target cells following the incubation of commercial CD45RA antibodies. This will help determine whether any commercial CD45RA antibodies could block the binding of CSC17.1, which could indicate closely located epitopes or steric hindrance effects. The following protocol was followed. CD45RA expressing cell line, KG1a (˜b 1×10⁵ ) was saturated (˜100 ug/mL)with an unlabeled commercial CD45RA antibody (clones used: MB1, MEM56, 4KB5, the F8-11-13 clone of CBL121, HI100, 6D36, 5B24) for one hour followed by a wash step to remove any unbound antibodies. Cells were then incubated with biotinylated CSC17.1 (1 ug/mL) for one hour followed by another wash step to remove unbound CSC17.1. The cells were then incubated with PE labeled streptavidin for 30 minutes followed by a final wash. Cells were then analyzed by FACs.

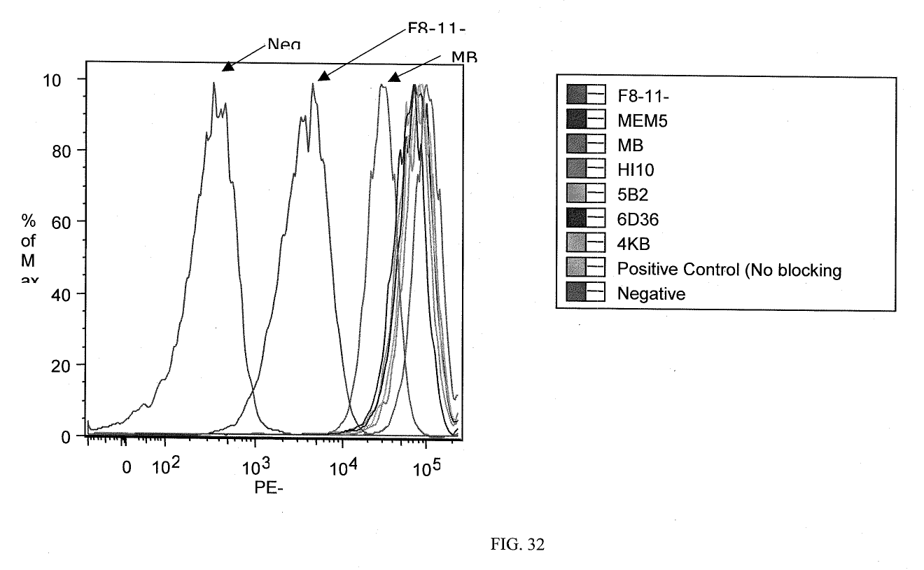

FIG. 32 shows a FACs histogram demonstrating that none of the commercial antibody fully inhibits the binding capability of CSC17.1. Five of the seven antibodies did not have any affect on the binding of CSC17.1 to KG1a (no difference from the positive control). Two antibodies had some effect in CSC17.1 binding. Blocking with clone MB1 had a slight effect (half log) on CSC17.1 while the F8-11-13 clone of CBL121 decreased CSC17.1 by about 50% (full log). For the positive control, no blocking antibody was used and for the negative control only PE labeled streptavidin was used.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the Claims appended hereto and their equivalents.

All patents, patent applications, publications, and references cited herein are expressly incorporated by reference to the same extent as if each individual publication or patent application was specifically and individually indicated to be incorporated by reference.

What is claimed is:

1. An antibody that specifically binds to CD45RA+ hematopoietic tumor cells (HTCs) and inhibits their proliferation and/or mediates their destruction.

2. The antibody according to claim 1 wherein said antibody is specifically immunoreactive with CD45RA+ HTCs and minimally crossreactive with hematopoietic stem cells (HSCs).

3. The antibody according to claim 1 or 2, wherein said antibody is an IgG isotype.

4. The antibody according to claim 1, wherein said antibody is a humanized antibody.

5. The antibody according to claim 4, wherein said humanized antibody is from a transgenic animal comprising a human immunoglobulin gene.

6. The antibody according to claim 1, wherein said antibody specifically binds a CD45RA epitope corresponding to SEQ ID NO: 1.

7. The antibody according to claim 6, wherein said antibody is the monoclonal antibody designated 17.1 produced by the hybridoma cell line deposited with the ATCC under Accession Number PTA-7339.

8. An antibody that specifically binds to an epitope that is specifically bound by the antibody of claim 7.

9. The hybridoma cell line designated Accession Number PTA 7339.

10. A method of inducing apoptosis in CD45RA+ hematopoietic tumor cells (HTCs) comprising contacting said HTCs with a composition comprising an anti-CD45RA antibody.

11. The method according to claim 10, wherein said contacting inhibits the proliferation of said CD45+ hematopoietic tumor cells (HTCs).

12. A method of treating a patient with a haematological proliferative disorder characterized by CD45RA+ HTCs comprising administering to said patient a composition comprising an anti-CD45RA antibody.

13. The method according to claim 12, wherein said administering depletes said CD45RA+ HTCs in said patient.

14. The method according to claim 10 or 12, wherein said anti-CD45RA antibody specifically binds a CD45RA epitope corresponding to SEQ ID NO:1.

15. The method according to claim 10 or 12, wherein said composition comprises an anti-CD45RA antibody complex.

16. The method according to claim 12, wherein said hematological proliferative disorder is a myoproliferative disorder.

17. The method according to claim 12, wherein said hematological proliferative disorder is a lymphoproliferative disorder.
